(19) United States
(12) Patent Application Publication
(10) Pub. No.: US 2023/0087600 A1
(43) Pub. Date: Mar. 23, 2023

(54) IL-10 AND USES THEREOF

(71) Applicant: Bristol-Myers Squibb Company, Princeton, NJ (US)

(72) Inventors: Shodeinde COKER, Princeton, NJ (US); Robert F. GRAZIANO, Frenchtown, NJ (US); Murali GURURAJAN, San Diego, CA (US); Kanstantsin V. KATLINSKI, Wayland, MA (US); James K. LOY, Gales Ferry, CT (US); Paul E. MORIN, Pennington, NJ (US); Brian A. POIRSON, Somerset, NJ (US); Vanessa M. SPIRES, Howell, NJ (US); Zheng YANG, Plainsboro, NJ (US)

(73) Assignee: BRISTOL-MYERS SQUIBB COMPANY, Princeton, NJ (US)

(21) Appl. No.: 17/797,666

(22) PCT Filed: Feb. 5, 2021
(86) PCT No.: PCT/US2021/016850

§ 371 Date: Aug. 4, 2022

Related U.S. Application Data
(60) Provisional application No. 62/970,957, filed on Feb. 6, 2020.

(51) Int. Cl. C07K 14/54; C07K 14/76; C12N 15/79; A61K 38/20; A61P 35/00

(52) U.S. Cl. CPC C07K 14/5428; C07K 14/76; C12N 15/79; A61K 38/2066; A61P 35/00; C07K 2319/30; C07K 2319/31

(57) ABSTRACT

The present disclosure provides fusion proteins comprising an IL-10 polypeptide and a second polypeptide, e.g., an Fc polypeptide. Certain aspects of the present disclosure are directed to methods of treating a subject comprising administering the IL-10 fusion protein. In certain aspects, the subject is afflicted with a cancer.

### CROSS-REFERENCE TO RELATED APPLICATIONS

This application claims the benefit under 35 U.S.C. § 119(e) of U.S. Provisional Application Ser. No. 62/970,957, filed Feb. 6, 2020; the disclosure of which is incorporated herein by reference.

### SEQUENCE LISTING

Incorporated herein by reference in its entirety is a Sequence Listing named “20210205_SEQL_13311WOPCT_ST25.txt,” comprising SEQ ID NO:1 through SEQ ID NO:45, which include nucleic acid and/or amino acid sequences disclosed herein. The Sequence Listing has been submitted herewith in ASCII text format via EFS-Web, and thus constitutes both the paper and computer readable form thereof. The Sequence Listing was created on Feb. 3, 2021, and is approximately 104 KB in size.

### BACKGROUND OF THE DISCLOSURE

IL-10 is a pleiotropic immunomodulatory cytokine produced by M [symbol] , B, NK, and T cells (CD4+, CD8+, and Tregs). IL-10 binds as a noncovalent homodimer with high affinity to the IL-10 receptor alpha (IL-10Ra) leading to recruitment of the IL-10 receptor beta (IL-10Rβ). Receptor binding activates a complex signaling cascade, including phosphorylation of STAT3 and STAT1. Signaling through this pathway can lead to both anti-inflammatory and pro-inflammatory effects on various immune cell subsets where the receptor is expressed. The pro-inflammatory effects include expansion, activation, and cytolytic potentiation of primed CD8⁺ T cells and NK cells. Comparatively, the anti-inflammatory effects include suppression of myeloid cytokine production and priming capacity. Treatment with an IL-10 molecule can induce the expansion and activation of tumor-specific CD8⁺ T and NK cells, driving IFNγ-dependent tumor killing mechanisms in solid tumors, and can have potential benefit in combination with IO agents or standard of care (Autio, et al., Current Oncology Reports, 2019; 2 1:19).

Despite its dual immunomodulatory roles, IL-10 has been identified as an anti-tumor agent. Early studies in the IL-10 knockout mouse revealed a strain-dependent prevalence for development of colon adenocarcinoma (Berg et al., J Clin Invest, 1996; 98:1010-1020) as well increased incidence of DMBA induced skin tumors with reduced T cells (Mumm, et al, Cancer cell, 2011; 20:781-796). Similarly, humans with deficiencies in IL-10 signaling through mutations in the IL-10 receptor develop lymphomas with a much lower frequency of infiltrating cytolytic T cells (Neven et al., Blood, 2013; 122:3713-3722). Beyond genetic evidence, therapeutic administration of recombinant IL-10 or pegylated IL-10 have also shown anti-tumor activity in several mouse models, where efficacy was shown to require CD8+ T cells and IFNγ-dependent upregulation of tumor MHC class I antigens (Mumm, et al, Cancer cell, 2011; 20:781-796). However, both in mouse and man, repeated daily dosing is often required for therapeutic activity due to the cytokine's short half-life. A pegylated human IL-10 (PEGIL-10), now in clinical trials, is showing encouraging clinical signals across several tumor indications, yet still requires daily dosing to maintain a pharmacokinetic (PK) profile needed for activity (Naing, et al., Cancer Cell, 2018; 34:775-791). Moreover, hematologic toxicity such as anemia and thrombocytopenia has been observed clinically with repeated daily dosing (Autio, et al., Current Oncology Reports, 2019; 2 1:19; Naing, et al., Journal of Clinical Oncology, 2016; 34, 3562-3569; Sosman, et al., British Journal of Haematology, 2000; 111(1), 104-111). Thus, a human IL-10 agonist that is efficacious with a less frequent dosage regimen is needed. Such less frequent dosing not only obviates the need for daily injection, but also facilitates recovery from hematologic toxicity between doses.

### SUMMARY OF THE DISCLOSURE

Certain aspects of the present disclosure are directed to an IL-10 fusion protein comprising (i) an IL-10 polypeptide, comprising an amino acid sequence having at least 95% sequence identity to the amino acid sequence set forth in SEQ ID NO: 1; and (ii) a second polypeptide, wherein the IL-10 fusion protein comprises an IL-10 activity. In some aspects, the second polypeptide comprises an albumin polypeptide. In some aspects, the second polypeptide comprises an Fc polypeptide. In some aspects, the Fc polypeptide comprises an amino acid sequence having at least about 95% sequence identity to an amino acid sequence selected from SEQ ID NOs: 4-12.

Certain aspects of the present disclosure are directed to an IL-10 fusion protein comprising (i) an IL-10 polypeptide, comprising an amino acid sequence having at least 95% sequence identity to the amino acid sequence set forth in SEQ ID NO: 1; and (ii) a second polypeptide comprising an Fc polypeptide, wherein the IL-10 fusion protein is capable of treating cancer in a subject in need thereof when the IL-10 fusion protein is administered to the subject no more than about once a week. In some aspects, the IL-10 fusion protein is capable of treating cancer in a subject in need thereof when the IL-10 fusion protein is administered to the subject no more than once about every two weeks. In some aspects, the IL-10 fusion protein is capable of treating cancer in a subject in need thereof when the IL-10 fusion protein is administered to the subject no more than once about every four weeks.

In some aspects, the second polypeptide is fused to the N-terminus of the IL-10 polypeptide. In some aspects, the second polypeptide is fused to the C-terminus of the IL-10 polypeptide.

In some aspects, the IL-10 polypeptide is fused to the second polypeptide by a linker. In some aspects, the linker comprises at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, or at least about 21 amino acids. In some aspects, the linker comprises at least about 15 amino acids. In some aspects, the linker comprises at least about 20 amino acids. In some aspects, the linker comprises at least about 21 amino acids.

In some aspects, the linker comprises a Glycine and a Serine. In some aspects, the linker comprises a GGGGS (SEQ ID NO: 39) motif or a GGGS (SEQ ID NO: 38) motif. In some aspects, the linker comprises an amino acid sequence selected from SEQ ID NOs: 38-45.

In some aspects, the IL-10 polypeptide comprises an amino acid sequence having at least about 96%, at least about 97%, at least about 98%, or at least about 99% sequence identity to the amino acid sequence set forth in SEQ ID NO: 1. In some aspects, the IL-10 polypeptide comprises the amino acid sequence set forth in SEQ ID NO: 1.

In some aspects, the Fc polypeptide comprises an amino acid sequence having at least about 96%, at least about 97%, at least about 98%, or at least about 99% sequence identity to an amino acid sequence selected from SEQ ID NOs: 4-12. In some aspects, the Fc polypeptide comprises an amino acid sequence selected from SEQ ID NOs: 4-12.

In some aspects, the IL-10 fusion protein comprises an amino acid sequence having at least 95% sequence identity to an amino acid sequence selected from SEQ ID NOs: 14-32. In some aspects, the IL-10 fusion protein comprises an amino acid sequence having at least 98% sequence identity to an amino acid sequence selected from SEQ ID NOs: 14-32. In some aspects, the IL-10 fusion protein comprises an amino acid sequence having at least 99% sequence identity to an amino acid sequence selected from SEQ ID NOs: 14-32. In some aspects, the IL-10 fusion protein comprises an amino acid sequence selected from SEQ ID NOs: 14-32 with 3 or fewer substitutions, insertions, or deletions. In some aspects, the IL-10 fusion protein comprises an amino acid sequence selected from SEQ ID NOs: 14-32 with 2 or fewer substitutions, insertions, or deletions. In some aspects, the IL-10 fusion protein comprises an amino acid sequence selected from SEQ ID NOs: 14-32 with 1 substitution, insertion, or deletion. In some aspects, the IL-10 fusion protein comprises an amino acid sequence selected from SEQ ID NOs: 14-32.

In some aspects, the IL-10 fusion protein comprises an amino acid sequence having at least 95% sequence identity to an amino acid sequence selected from SEQ ID NOs: 33-36. In some aspects, the IL-10 fusion protein comprises an amino acid sequence having at least 98% sequence identity to an amino acid sequence selected from SEQ ID NOs: 33-36. In some aspects, the IL-10 fusion protein comprises an amino acid sequence having at least 99% sequence identity to an amino acid sequence selected from SEQ ID NOs: 33-36. In some aspects, the IL-10 fusion protein comprises an amino acid sequence selected from SEQ ID NOs: 33-36 with 3 or fewer substitutions, insertions, or deletions. In some aspects, the IL-10 fusion protein comprises an amino acid sequence selected from SEQ ID NOs: 33-36 with 2 or fewer substitutions, insertions, or deletions. In some aspects, the IL-10 fusion protein comprises an amino acid sequence selected from SEQ ID NOs: 33-36 with 1 substitution, insertion, or deletion. In some aspects, the IL-10 fusion protein comprises an amino acid sequence selected from SEQ ID NOs: 33-36.

In some aspects, the IL-10 fusion protein comprises an IL-10 dimer comprising, a first polypeptide and a second polypeptide, wherein the first polypeptide comprises an IL-10 fusion protein disclosed herein, and wherein the second polypeptide comprises a second Fc polypeptide. In some aspects, the second polypeptide comprises a second IL-10 polypeptide fused to the second Fc polypeptide.

In some aspects, the IL-10 dimer is a homodimer. In some aspects, the first polypeptide comprises the amino acid sequence set forth in SEQ ID NO: 1 and the second polypeptide comprises the amino acid sequence set forth in SEQ ID NO: 1. In some aspects, the first polypeptide comprises an amino acid sequence selected from SEQ ID NOs: 14-36 and the second polypeptide comprises an amino acid sequence selected from SEQ ID NOs: 14-36.

In some aspects, the IL-10 dimer is a heterodimer.

In some aspects, the first polypeptide and the second polypeptide are linked by a covalent bond. In some aspects, the first polypeptide and the second polypeptide are linked by a disulfide bond. In some aspects, the first polypeptide and the second polypeptide are linked by a peptide bond. In some aspects, the first polypeptide and the second polypeptide are linked by a peptide linker. In some aspects, the peptide linker is a cleavable linker.

Certain aspects of the present disclosure are directed to a polynucleotide or a set of polynucleotides encoding an IL-10 fusion protein disclosed herein.

Certain aspects of the present disclosure are directed to a vector or a set of vectors comprising a polynucleotide or a set of polynucleotides disclosed herein. In some aspects, the vector is a viral vector.

Certain aspects of the present disclosure are directed to a host cell comprising an IL-10 fusion protein disclosed herein, a polynucleotide or a set of polynucleotides disclosed herein, or a vector or a set of vectors disclosed herein. In some aspects, the host cell is a mammalian cell. In some aspects, the host cell is selected from a Chinese hamster ovary (CHO) cell, an HEK293 cell, a BHK cell, a murine myeloma cell (NS0 and Sp2/0), a monkey kidney (COS) cell, a VERO cell, a fibrosarcoma HT-1080 cell, and a HeLa cell.

Certain aspects of the present disclosure are directed to a pharmaceutical composition comprising an IL-10 fusion protein disclosed herein, a polynucleotide or a set of polynucleotides disclosed herein, or a vector or a set of vectors disclosed herein, and a pharmaceutically acceptable excipient.

Certain aspects of the present disclosure are directed to a method of treating a cancer in a subject in need thereof, comprising administering to the subject an effective amount of an IL-10 fusion protein disclosed herein, a polynucleotide or a set of polynucleotides disclosed herein, a vector or a set of vectors disclosed herein, or a pharmaceutical composition disclosed herein.

Certain aspects of the present disclosure are directed to a method of killing a cancer cell in a subject in need thereof, comprising administering to the subject an effective amount of an IL-10 fusion protein disclosed herein, a polynucleotide or a set of polynucleotides disclosed herein, a vector or a set of vectors disclosed herein, or a pharmaceutical composition disclosed herein.

Certain aspects of the present disclosure are directed a method of treating cancer in a subject in need thereof, comprising administering to the subject an effective amount of an IL-10 fusion protein at a dosing interval of at least about 7 days, wherein the IL-10 fusion protein comprises an IL-10 polypeptide and a second polypeptide, which comprises an albumin polypeptide or an Fc polypeptide.

Certain aspects of the present disclosure are directed a method of killing a cancer call in a subject in need thereof, comprising administering to the subject an effective amount of an IL-10 fusion protein at a dosing interval of at least about 7 days, wherein the IL-10 fusion protein comprises an IL-10 polypeptide and a second polypeptide, which comprises an albumin polypeptide or an Fc polypeptide.

In some aspects, the second polypeptide is an albumin polypeptide. In some aspects, the second polypeptide is an Fc polypeptide. In some aspects, the IL-10 fusion protein further comprises a linker. In some aspects, the linker comprises a linker disclosed herein.

In some aspects, the IL-10 fusion protein is administered at a dosing interval of at least about 7 days, at least about 10 days, at least about 14 days, at least about 17 days, at least about 21 days, at least about 24 days, or at least about 28 days. In some aspects, the IL-10 fusion protein is administered no more than once week. In some aspects, the IL-10 fusion protein is administered no more than once every 2 weeks. In some aspects, the IL-10 fusion protein is administered no more than once every 3 weeks. In some aspects, the IL-10 fusion protein is administered no more than once every 4 weeks. In some aspects, the IL-10 fusion protein is administered at a dosing interval of at least about 7 days to at least about 28 days. In some aspects, the IL-10 fusion protein is administered at a dosing interval of at least about 14 days. In some aspects, the IL-10 fusion protein is administered at a dosing interval of at least about 21 days. In some aspects, the IL-10 fusion protein is administered at a dosing interval of at least about 28 days. In some aspects, the IL-10 fusion protein is administered about once a week. In some aspects, the IL-10 fusion protein is administered once about every 2 weeks. In some aspects, the IL-10 fusion protein is administered once about every 3 weeks. In some aspects, the IL-10 fusion protein is administered once about every 4 weeks. In some aspects, the IL-10 fusion protein is administered once about every 6 weeks. In some aspects, the IL-10 fusion protein is administered once about every 2 months.

In some aspects, the IL-10 fusion protein is administered as a single dose. In some aspects, the effective amount of the IL-10 fusion protein consists essentially of or consists of a single dose.

In some aspects, the IL-10 fusion protein comprises an amino acid sequence having at least 99% sequence identity to an amino acid sequence selected from SEQ ID NOs: 14-32, and wherein the IL-10 fusion protein is administered at a dosing interval of at least about 2 weeks. In some aspects, the IL-10 fusion protein comprises an amino acid sequence selected from SEQ ID NOs: 14-32. In some aspects, the IL-10 fusion protein comprises of SEQ ID NO: 14. In some aspects, the IL-10 fusion protein is administered once every 2 weeks. In some aspects, the IL-10 fusion protein is administered about once every 3 weeks. In some aspects, the IL-10 fusion protein is administered once about every 4 weeks. In some aspects, the IL-10 fusion protein is administered once every 5 weeks. In some aspects, the IL-10 fusion protein is administered once about every 6 weeks.

In some aspects, the cancer comprises a tumor. In some aspects, the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma, HCC), breast cancer, colon carcinoma, head and neck cancer (or carcinoma), head and neck squamous cell carcinoma (HNSCC), gastric cancer, germ cell tumor, pediatric sarcoma, sinonasal natural killer/T-cell lymphoma, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, and combinations of said cancers.

In some aspects, the cancer is selected from acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

In some aspects, the cancer is selected from RCC, NSCLC, gastric cancer, HCC, Squamous cell carcinoma of the head and neck (SCCHN), and any combinations of said cancers. In some aspects, the cancer is selected from RCC, NSCLC, gastric cancer, SCCHN, and any combinations of said cancers. In some aspects, the cancer is selected from melanoma, bladder cancer, pancreatic cancer, colon cancer, SCLC, mesothelioma, hepatocellular carcinoma, prostate cancer, multiple myeloma, and combinations of said cancers.

In some aspects, the method further comprises administering to the subject a second anticancer therapy. In some aspects, the second anticancer therapy comprises a therapy selected from the group consisting of an immunotherapy, a chemotherapy, a radiation therapy, a surgery, an agent that activates innate immune cells, an agent that enhances survival of NK and/or CD8+ T-cells, an agent that inhibits Tregs (T regulatory cells), TAMs (tumor-associated macrophages), CAFs (cancer-associated fibroblasts), or MDSCs (myeloid-derived suppressor cells), and any combination thereof. In some aspects, the second anticancer therapy comprises an effective amount of an antibody or an antigen-binding fragment thereof that specifically binds a protein selected from Inducible T cell Co-Stimulator (ICOS), CD137 (4-1BB), CD134 (OX40), NKG2A, CD27, CD38, CD73, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, SLAMF7, BCMA, MICA, MICB, CCR8, and any combination thereof.

In some aspects, the second anticancer therapy comprises an antibody or antigen-binding fragment thereof that specifically binds PD-1 (“an anti-PD-1 antibody”). In some aspects, the anti-PD-1 antibody comprises nivolumab or pembrolizumab.

In some aspects, the second anticancer therapy comprises an antibody or an antigen-binding fragment thereof that specifically binds PD-L1 (“an anti-PD-L1 antibody”). In some aspects, the anti-PD-L1 antibody is selected from atezolizumab, durvalumab, and avelumab.

In some aspects, the second anticancer therapy comprises an antibody or an antigen-binding fragment thereof that specifically binds CTLA-4 (“an anti-CTLA-4 antibody”). In some aspects, the anti-CTLA-4 antibody comprises tremelimumab or ipilimumab.

In some aspects, the second anticancer therapy comprises an antibody or an antigen-binding fragment thereof that specifically binds CTLA-4, e.g., tremelimumab or ipilimumab, and an antibody or antigen-binding fragment thereof that specifically binds PD-1, e.g., nivolumab or pembrolizumab. In some aspects, the second anticancer therapy comprises an antibody or an antigen-binding fragment thereof that specifically binds CTLA-4, e.g., tremelimumab or ipilimumab, and an antibody or antigen-binding fragment thereof that specifically binds PD-L1, e.g., atezolizumab, durvalumab, or avelumab.

In some aspects, the second anticancer therapy comprises a chemotherapy selected from a proteasome inhibitor, an IMiD, a Bet inhibitor, an IDO antagonist, a platinum-based chemotherapy, STING agonists, NLRP3 agonists, TLR7 agonists, and any combination thereof.

In some aspects, the second therapy comprises an agent elected from doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, Folinic acid (leucovorin), Fluorouracil (5-FU), Oxaliplatin (Eloxatin), FOLFOX, Paclitaxel, Docetaxel, vincristine (ONCOVIN®), fludarabine (FLUDARA®), thalidomide (THALOMID®), alemtuzumab (CAMPATH®, ofatumumab (ARZERRA®), everolimus (AFINITOR®, ZORTRESS®), and carfilzomib (KYPROLISTM).

In some aspects, the second anticancer therapy comprises an agent that enhances survival of NK and/or CD8+ T-cells selected from an agent comprising IL-2, such as pegylated IL-2, IL-18, and IL-15.

In some aspects, the second anticancer therapy comprises a CAR-T therapy, such as CD19-targeted CAR-T.

In some aspects, the second anticancer therapy comprises a bispecific antibody therapy, such as CD3-targeted biospecific antibody, e.g., anti-CD3/CD20, anti-CD3/BCMA biospecifics.

In some aspects, the second anticancer therapy comprises a standar-of-care therapy, such as an anti-angiogenic therapy (e.g., Bevacuzimab, Sorafinib etc), or radiation.

Certain aspects of the present disclosure are directed to a method of preparing an IL-10 fusion protein, comprising expressing a polynucleotide or a set of polynucleotides disclosed herein or a vector or a set of vectors disclosed herein in a host cell under suitable conditions. In some aspects, the method further comprises collecting the IL-10 fusion protein.

### BRIEF DESCRIPTION OF THE DRAWINGS/FIGURES

FIGS. 1A-1B shows the amino acid sequence of Fc-IL-10 comprising SEQ ID NO: 14 (1A) and a diagram model of Fc-IL-10 (1B), which is a 90 kDa Fc Fusion of wild-type human IL-10 to the c-terminus of the human IgG1.3f Fc domain.

FIGS. 2A-2B show IFNγ (pg/mL) level secreted by pre-activated human primary CD8+ T cells which were treated with Fc-IL-10, recombinant IL-10, or PEG-IL-10 for 72 hours with (FIG. 2B) or without IL2 (FIG. 2A). IFNγ (pg/mL) was measured from the supernatant by AlphaLISA.

FIGS. 2C-2D show cytotoxicity, as measured by percentage cell lysis, mediated by primary human NK cells pretreated with recombinant human IL-10 or Fc-IL-10 at 10 nM. K562 target cells were added at an effector to target (E:T) ratio of 20:1 (FIG. 2C) or 5:1 (FIG. 2D). Data representative of two experiments with 2-3 donors per experiment.

FIGS. 3A-3F show induction of Granzyme B (FIGS. 3C and 3F) and IFNγ (FIGS. 3B and 3E) gene expression by Fc-IL-10 in mouse (FIGS. 3A-3C) and human (FIGS. 3D-3F) CRC tumor explants. Mouse MC38 or human CRC tumors chopped to small chunks were cultured for 72 hours in media containing IL-2 with or without the addition of 0.1 nM (m)Fc-IL-10 and then subjected to transcriptional analsis. Representative transcripts for CD8a (FIGS. 3A and 3D), IFNγ (FIGS. 3B and 3E), and Granzyme B (FIGS. 3C and 3F) are plotted below as fold change over control. Each error bar represents SEM of 4 replicate measurements of the sample pooled from 8 wells per treatment.

FIGS. 4A-4F show monotherapy effects of mFc-mIL-10 in single dose, titration studies in MC38 tumor models. 1E6 MC38 tumor cells were implanted subcutaneously on day 0 in C57BL6 female mice. Tumors are measured and randomized at 100 mm³ tumor volume into groups for treatment. mFc-mIL-10 was given as a single titrated dose at day 7 (TV=100 mm³) intraperitoneally ranging from 10-0.1 mg/kg. Tumor volume was measured and number of tumor free (TF) mice were tracked. MOPC-21 was used as isotype control. “1/10 TF” means one out of a total of 10 mice was tumor free.

FIGS. 5A-5K show monotherapy effects of mFc-mIL-10 or PEG-mIL-10 in single dose, titration studies in the MC38 tumor model. MC-38 tumor bearing C57BL/6NCrl female mice received a single IP administration on day 6 of: 0.1-10 mg/kg mFc-IL-10, or equivalent IL-10 molar concentration of 10 kD PEG-mIL10, respectively, or isotype control anti-DT mIgG1 D265A. Tumor volume was measured and number of tumor free (TF) mice were tracked.

FIGS. 6A-6B show proliferation and activation of tumor CD8+ T cells (FIG. 6A) and NK cells (FIG. 6B) at day 5 post dose in MC38 tumors, measured by flow cytometry as percent positive of Ki67 and Granzyme B. Error bars represent standard deviation for each group. ****p<0.0001, 10 mice per group.

FIGS. 7A-7E show the effects of single dose mFc-mIL-10 in combination with anti-PD-1 in the CT26 tumor model. CT26 tumor cells were implanted subcutaneously on day 0 in BALB/c female mice. Anti-PD-1 was dosed intraperitoneally every 4 days for 3 doses (Q4Dx3) at 10 mg/kg, starting on day 7. mFC-mIL-10 was given as a single titrated dose at day 7 ranging from 1-0.1 mg/kg. Tumors were measured and number of tumor free (TF) mice were tracked.

FIG. 7F shows observed drug concentration-time profile of mFc-mIL-10 in CT26 tumor bearing mice (n=4-6 mice per dose group). The drug levels (mean drug concentration±SD) were below the lower limit of quantification (LLOQ) 14 days after dosing in all of the groups.

FIGS. 7G-7I show percentage of tumor specific AH1 tetramer positive CD8+ T cells in CT26 tumors of treated mice at 7 (FIG. 7G), 14 (FIG. 7H), and 21 (FIG. 7I) days post dosing the combination of mFc-mIL-10 and anti-PD-1. Error bars represent standard deviation for each group. Day 7 is representative of three experiments, day 14 representative of two experiments and day 21 a single experiment. * p<0.05.

FIGS. 8A-8H show the effects of single low dose of mFc-IL-10, a single high dose of PEG-mIL-10 (3.0 mg/kg) or 25 daily dose of PEG-mIL-10 (0.2 or 1.0 mg/kg 5 kD), in combination with anti-PD-1 in the CT26 tumor model. 1E6 CT26 tumor cells were implanted subcutaneously (SC) on day 0 in BALB/c female mice. Anti-PD-1 was dosed intraperitoneally (IP) every 4 days for 3 doses (Q4Dx3) at 10 mg/kg, starting on day 7. mFC-mIL-10 in combination with anti-PD-1, was given as a single dose at day 7 intraperitoneal at 0.03, 0.1 or 0.3 mg/kg. 5 kDa PEGmIL-10, in combination with anti-PD-1, was dosed daily for 25 days starting at day 7 at 0.2 or 1 mg/kg IP or given as a single dose of 3 mg/kg IP on day 7 kg.

FIG. 9A shows drug concentration-time profile with fitted curves of mFc-mIL-10 following IP administration at 0.03, 0.1 or 0.3 mg/kg single dose to mice bearing the CT26 tumor. FIG. 9B shows the drug concentration-time profile with fitted curves of pegylated mIL-10, administered daily SC for 25 days at 0.2 mg/kg or in a single SC dose at 3 mg/kg. The symbols represent the observed data points while the lines are model-fitted results.

FIG. 10A shows percent survival and FIGS. 10B-10E show percent weight loss in mice with Azoxymethane (AOM)/dextran sulfate sodium (DSS)-induced colitis and colon tumors. The mice were treated with isotype control (anti-DT mIgG1+anti-DT mIgG2a) (FIGS. 10A and 10B), anti-CTLA4 (FIGS. 10A and 10D), mFc-IL-10 (FIGS. 10A and 10C), or a combination of anti-CTLA4 and mFc-IL-10 (FIGS. 10A and 10E). Percent weight loss relative to baseline was used as a surrogate measure of colitis severity.

FIGS. 11A-11C show tumor analysis in mice treated with isotype control (n=8) and mFc-IL-10 (n=9), respectively. In FIG. 11A, the dots represent the sizes of individual lesions; in FIG. 11B, each dot represents the number of lesions in an individual animal; and in FIG. 11C, each dot represents the cumulative size of all tumors in an individual animal. **p-value <0.05.

### DETAILED DESCRIPTION OF DISCLOSURE

Certain aspects of the present disclosure are directed to an IL-10 fusion protein comprising (i) an IL-10 polypeptide and an Fc polypeptide, wherein the IL-10 fusion protein comprises an IL-10 activity. Other aspects of the present disclosure are directed to methods of treating a disease or condition, e.g., a cancer, in a subject in need thereof comprising administering an IL-10 fusion protein disclosed herein.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a composition comprising a therapeutic agent to a subject, using any of the various methods and delivery systems known to those skilled in the art. Examples of routes of administration for the IL-10 fusion proteins and IL-10 dimers disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “antibody” (Ab) includes, without limitation, a glycoprotein immunoglobulin which binds specifically to an antigen and comprises at least two heavy (H) chains and two light (L) chains interconnected by disulfide bonds, or an antigen-binding portion thereof. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The term “Fc,” “Fc polypeptide,” “Fc domain,” or “Fc region” refers to an Fc domain of an antibody, or fragment thereof. An Fc may be a native Fc region comprising an amino acid sequence identical to the amino acid sequence of an Fc region found in nature, or a variant Fc region comprising an amino acid sequence which differs from that of a native Fc region by virtue of at least one amino acid. References made to amino acid numbering of immunoglobulins or immunoglobulin fragments, or regions, are all based on Kabat et al. 1991, Sequences of Proteins of Immunological Interest, U. S. Department of Public Health, Bethesda; MD, incorporated herein by reference in its entirety. An Fc can comprise the CH2 and CH3 domains of an immunoglobulin with or without the hinge region of the immunoglobulin. Exemplary Fc variants are provided in WO 2004/101740 and WO 2006/074199, incorporated herein by reference in its entirety.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of an IL-10 polypeptide and an Fc polypeptide. A fusion protein is created, for example, by chemical synthesis, or by creating and translating a polynucleotide in which the peptide regions are encoded in the desired relationship. A fusion protein can comprise a second amino acid sequence linked to the first amino acid sequence by a peptide, a polypeptide, or a peptide bond, covalent bond, non-peptide bond, or a non-covalent bond.

The terms “linked” and “fused” as used herein interchangeably refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence is linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein the term “associated with” refers to a covalent or non-covalent bond formed between a first amino acid chain and a second amino acid chain. In one embodiment, the term “associated with” means a covalent, non-peptide bond or a non-covalent bond. This association can be indicated by a colon, i.e., (:). In another embodiment, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system).

“Dosing interval,” as used herein, means the time interval between doses. The dosing interval may be, for example, a day, 2 days, 3 days, 7 days (a week), 2 weeks, 3 weeks, 4 weeks, 6 weeks, 8 weeks, and so on.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

Also included in the present invention are fragments or variants of polypeptides, and any combination thereof. The term “fragment” or “variant” when referring to polypeptides used in the methods of the present disclosure include any polypeptides which retain at least some of the properties of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules used in the methods of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative.

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison, the target sequence or the reference sequence, and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one embodiment, the polynucleotide variants contain alterations which produce silent substitutions, additions, or deletions, but do not alter the properties or activities of the encoded polypeptide. In another embodiment, nucleotide variants are produced by silent substitutions due to the degeneracy of the genetic code. In other embodiments, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. See Ron et al., J. Biol. Chem. 268: 2984-2988 (1993), and Dobeli et al., J. Biotechnology 7:199-216 (1988), both incorporated herein by reference in their entirety. Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a and found that “[m]ost of the molecule could be altered with little effect on either [binding or biological activity].” (See Abstract.)

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

The term “weight-based dose” as referred to herein means that a dose that is administered to a patient is calculated based on the weight of the patient. The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent. The use of the term “fixed dose” with regard to a method of the disclosure means that two or more different agents, for example, Fc-IL-10 and a second therapeutic agent (e.g., an antibody), in a single composition are present in the composition in particular (fixed) ratios with each other. In some aspects, the fixed dose is based on the weight (e.g., mg) of the agents. In certain aspects, the fixed dose is based on the concentration (e.g., mg/ml) of the agents. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first agent (e.g., an IL-10 fusion protein) to mg second antibody (e.g., an additional anticancer therapy).

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in progression-free or overall survival, an increase in frequency and duration of disease symptom-free or progression-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In some aspects, a therapeutically effective amount of the drug promotes cancer regression to the point of eliminating the cancer. “Promoting cancer regression” means that administering an effective amount of the drug, alone or in combination with a second anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, immunogenicity, or other adverse physiological effects at the cellular, organ and/or organism level (adverse effects) resulting from administration of the drug.

By way of example for the treatment of tumors, a therapeutically effective amount of an anti-cancer agent may inhibit cell growth or tumor growth by at least about 20%, more preferably by at least about 40%, even more preferably by at least about 60%, and still more preferably by at least about 80% relative to untreated subjects. In other aspects of the disclosure, tumor regression can be observed and continue for a period of at least about 20 days, at least about 40 days, or at least about 60 days. In some aspects, a therapeutically effective amount of an anti-cancer agent may kill tumor cells.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with a second anti-cancer agent, results in a reduction in tumor growth or size, necrosis of the tumor, a decrease in severity of at least one disease symptom, an increase in frequency and duration of disease symptom-free periods, an increase in progression-free survival or overall survival, a prevention of impairment or disability due to the disease affliction, or otherwise amelioration of disease symptoms in the patient.

The terms “once about every week,” “once about every two weeks,” or any other similar dosage regimen terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some aspects, a dosing frequency of once about every six weeks or once about every twelve weeks means that the first dose can be administered any day in the first week, and then the next dose can be administered any day in the sixth or twelfth week, respectively. In other aspects, a dosing frequency of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of can mean within 1 or more than 1 standard deviation per the practice in the art. Alternatively, “about” or “comprising essentially of can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

### II. Fusion Proteins

Certain aspects of the present disclosure are directed to IL-10 fusion proteins, comprising an IL-10 polypeptide and a second polypeptide. In some aspects, the second polypeptide may be an Fc polypeptide. In some aspects, the second polypeptide may be an albumin. In some aspects, the IL-10 fusion protein is capable of inducing interferon gamma (IFNγ), e.g., in human CD8⁺ T cells. In some aspects, the IL-10 fusion protein is capable of inducing cell-mediated cytotoxicity of human NK cells. In some aspects, the IL-10 fusion protein is capable of forming a dimer, e.g., a homodimer with a second IL-10 fusion protein or a heterodimer with a hIL-10 protein. In some aspects, the IL-10 fusion protein is capable of binding an IL-10 receptor. In some aspects, the fusion protein is capable of activating Jak1. In some aspects, the IL-10 fusion protein is capable of activating Tyk2. In some aspects, the IL-10 fusion protein is capable of activating STAT1. In some aspects, the IL-10 fusion protein is capable of activating STATS. In some aspects, the IL-10 fusion protein is capable of activating STATS. In some aspects, the IL-10 fusion protein is capable of eliciting an anti-inflammatory response. In some aspects, the IL-10 fusion protein is capable of eliciting a pro-inflammatory response. In some aspects, the IL-10 fusion protein is an IL-10 and Fc polypeptide fusion. Any IL-10 polypeptide and/or Fc polypeptide known in the art can be used in the fusion proteins disclosed herein. In some aspects, the IL-10 fusion protein is an IL-10 and albumin fusion. Any IL-10 polypeptide and/or albumin polypeptide known in the art can be used in the fusion proteins disclosed herein.

### II.A. IL-10 Polypeptides

As used herein, unless otherwise indicated “interleukin-10” and “IL-10” can refer to human IL-10 (“hIL-10; Genbank Accession Nos. NP_000563; M37897; NM_000572; UniProt—P22301; or U.S. Pat. No. 6,217,857) or mouse IL-10 (“mIL-10”). hIL-10 is expressed as a 178 amino-acid long protein, including an 18 amino acid signal peptide. Mature hIL-10 protein (SEQ ID NO 1; Table 1) has 160 amino acids. Although there is 80% homology between hIL-10 and mIL-10, only hIL-10 acts on both human and mouse cells, whereas mIL-10 has species specificity activity.

TABLE 1
IL-10 Sequences.

| SEQ | | |
|---|---|---|
| ID | | |
| NO | Description | Sequence |
| 1 | hIL-10 | SPGQGTQSENSCTHFPGNLPNMLRDLRDAFSRVKTFFQMKDQLDNLLLKESLLED |
| | Protein | FKGYLGCQALSEMIQFYLEEVMPQAENQDPDIKAHVNSLGENLKTLRLRLRRCHR |
| | | FLPCENKSKAVEQVKNAFNKLQEKGIYKAMSEFDIFINYIEAYMTMKIRN |
| 2 | hIL-10 | ACACATCAGGGGCTTGCTCTTGCAAAACCAAACCACAAGACAGACTTGCAAAAGA |
| | Nucleotide | AGGCATGCACAGCTCAGCACTGCTCTGTTGCCTGGTCCTCCTGACTGGGGTGAGG |
| | | GCCAGCCCAGGCCAGGGCACCCAGTCTGAGAACAGCTGCACCCACTTCCCAGGCA |
| | | ACCTGCCTAACATGCTTCGAGATCTCCGAGATGCCTTCAGCAGAGTGAAGACTTT |
| | | CTTTCAAATGAAGGATCAGCTGGACAACTTGTTGTTAAAGGAGTCCTTGCTGGAG |
| | | GACTTTAAGGGTTACCTGGGTTGCCAAGCCTTGTCTGAGATGATCCAGTTTTACC |
| | | TGGAGGAGGTGATGCCCCAAGCTGAGAACCAAGACCCAGACATCAAGGCGCATGT |
| | | GAACTCCCTGGGGGAGAACCTGAAGACCCTCAGGCTGAGGCTACGGCGCTGTCAT |
| | | CGATTTCTTCCCTGTGAAAACAAGAGCAAGGCCGTGGAGCAGGTGAAGAATGCCT |
| | | TTAATAAGCTCCAAGAGAAAGGCATCTACAAAGCCATGAGTGAGTTTGACATCTT |
| | | CATCAACTACATAGAAGCCTACATGACAATGAAGATACGAAACTGAGACATCAGG |
| | | GTGGCGACTCTATAGACTCTAGGACATAAATTAGAGGTCTCCAAAATCGGATCTG |
| | | GGGCTCTGGGATAGCTGACCCAGCCCCTTGAGAAACCTTATTGTACCTCTCTTAT |
| | | AGAATATTTATTACCTCTGATACCTCAACCCCCATTTCTATTTATTTACTGAGCT |
| | | TCTCTGTGAACGATTTAGAAAGAAGCCCAATATTATAATTTTTTTCAATATTTAT |
| | | TATTTTCACCTGTTTTTAAGCTGTTTCCATAGGGTGACACACTATGGTATTTGAG |
| | | TGTTTTAAGATAAATTATAAGTTACATAAGGGAGGAAAAAAAATGTTCTTTGGGG |
| | | AGCCAACAGAAGCTTCCATTCCAAGCCTGACCACGCTTTCTAGCTGTTGAGCTGT |
| | | TTTCCCTGACCTCCCTCTAATTTATCTTGTCTCTGGGCTTGGGGCTTCCTAACTG |
| | | CTACAAATACTCTTAGGAAGAGAAACCAGGGAGCCCCTTTGATGATTAATTCACC |
| | | TTCCAGTGTCTCGGAGGGATTCCCCTAACCTCATTCCCCAACCACTTCATTCTTG |
| | | AAAGCTGTGGCCAGCTTGTTATTTATAACAACCTAAATTTGGTTCTAGGCCGGGC |
| | | GCGGTGGCTCACGCCTGTAATCCCAGCACTTTGGGAGGCTGAGGCGGGTGGATCA |
| | | CTTGAGGTCAGGAGTTCCTAACCAGCCTGGTCAACATGGTGAAACCCCGTCTCTA |
| | | CTAAAAATACAAAAATTAGCCGGGCATGGTGGCGCGCACCTGTAATCCCAGCTAC |
| | | TTGGGAGGCTGAGGCAAGAGAATTGCTTGAACCCAGGAGATGGAAGTTGCAGTGA |
| | | GCTGATATCATGCCCCTGTACTCCAGCCTGGGTGACAGAGCAAGACTCTGTCTCA |
| | | AAAAATAAAAATAAAAATAAATTTGGTTCTAATAGAACTCAGTTTTAACTAGAAT |
| | | TTATTCAATTCCTCTGGGAATGTTACATTGTTTGTCTGTCTTCATAGCAGATTTT |
| | | AATTTTGAATAAATAAATGTATCTTATTGAGATGA |
| 3 | Signal | MHSSALLCCLVLLTGVRA |
| | Peptide | |

Homodimeric IL-10 binds to a single class of cell-surface receptors (IL-10R) which are primarily expressed by hematopoietic cells such as B cells, T cells, NK cells, monocytes, and macrophages. There is little to no expression found outside hematopoietic cells. Functional IL-10R complexes are tetramers consisting of two IL-10R1 polypeptide chains and two IL-10R2 chains. IL-10/IL-10R interaction activates the tyrosine kinases Jak1 and Tyk2, which are associated with IL-10R1 and IL-10R2, respectively. The receptor engagement and tyrosine phosphorylation activates the cytoplasmically localized inactive transcription factors STAT 1, 3, and 5, resulting in translocation and gene activation. Signaling through this pathway can lead to both anti-inflammatory and pro-inflammatory effects. IL-10 has been linked to a broad range of diseases, disorders, and conditions, including inflammatory conditions, immune-related disorders, fibrotic disorders, and cancer.

IL-10 has a relatively short in vivo serum half-life. For example, the half-life in mice, as measured by in vitro bioassay or by efficacy in the septic shock model system, is about 2 to 6 hours. In vivo loss of IL-10 activity may be due to several factors, including renal clearance, proteolytic degradation and monomerization in the blood stream. Because of its relatively short half-life, IL-10 has been conjugated to various partners, including polyethylene glycol.

PEGylation of a protein can increase its serum half-life by limiting renal clearance, as the PEG moiety adds considerable hydrodynamic radius to the protein. However, the conventional PEGylation methodologies are directed to monomeric proteins and larger, disulfide bonded complexes, e.g., monoclonal antibodies. Other cytokines, in addition to IL-10, have also been PEGylated, generally via monoPEGylation, e.g., PEG molecules attached to a single residue on the cytokine protein.

PEGylation of IL-10 presents problems not encountered with other PEGylated proteins, since the IL-10 dimer is held together by non-covalent interactions. Dissociation of IL-10, which may be enhanced during PEGylation, produces PEGylated IL-10 monomers, but these monomers do not retain biological activity of IL-10. Additionally, monoPEGylation on one IL-10 subunit leads to a non-homogenous mix of diPEGylated, monoPEGylated and nonPEGylated IL-10 molecules due to subunit shuffling. Furthermore, allowing a PEGylation reaction to proceed to completion will also permit non-specific and multi-PEGylated target proteins, thus reducing the bioactivity of these proteins. Thus, in comparison, fusion proteins that preserve the homodimer structure and can be easily produced homogeneously are advantageous.

Some aspects of the present disclosure are directed to fusion proteins comprising an IL-10 polypeptide and an Fc polypeptide. Any IL-10 polypeptide known in the art can be used in the fusion proteins described herein. In some aspects, the IL-10 polypeptide comprises hIL-10 or a variant thereof. In some aspects, the IL-10 polypeptide comprises murine IL-10 or a variant thereof. In some aspects, the IL-10 polypeptide comprises a non-human primate IL-10 or a variant thereof.

In some aspects, the IL-10 polypeptide comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 1. In some aspects, the IL-10 polypeptide comprises an amino acid sequence having at least about 98% sequence identity to the amino acid sequence set forth in SEQ ID NO: 1. In some aspects, the IL-10 polypeptide comprises an amino acid sequence having at least about 99% sequence identity to the amino acid sequence set forth in SEQ ID NO: 1. In some aspects, the IL-polypeptide comprises the amino acid sequence set forth in SEQ ID NO: 1 with 10 or fewer, 9 or fewer, 8 or fewer, 7 or fewer, 6 or fewer, 5 or fewer, 4 or fewer, 3 or fewer, or 2 or fewer substitutions, insertions, or deletions. In some aspects, the IL-polypeptide comprises the amino acid sequence set forth in SEQ ID NO: 1 with 2 or fewer substitutions, insertions, or deletions. In certain aspects, the IL-10 polypeptide comprises the amino acid sequence set forth in SEQ ID NO: 1.

In some aspects, the IL-10 polypeptide comprises a signal peptide. In some aspects, the signal peptide is fused to the N-terminus of the IL-10 polypeptide. In some aspects, the signal peptide comprises an amino acid having at least about 80%, at least about 85%, at least about 90%, at least about 95%, at least about 96%, at least about 97%, at least about 98%, or at least about 99% sequence identity with the amino acid sequence set forth in SEQ ID NO: 3. In some aspects, the signal peptide comprises the amino acid sequence set forth in SEQ ID NO: 3.

### II.B. Fc Polypeptides

Certain aspects of the present disclosure are directed to fusion proteins, comprising an Fc domain or a portion thereof and an IL-10 polypeptide. The Fc domain or a portion thereof can improve pharmacokinetic or pharmacodynamic properties of the fusion protein. In certain aspects, the Fc domain or a portion thereof extends a half-life of a molecule fused to the Fc domain or a portion thereof.

As used herein, the term “Fc domain” or “Fc region,” used interchangeably herein, refers to an FcR (e.g., FcRn) binding partner, or a mutant version thereof, e.g., a mutant Fc that has reduced binding to FcR and/or reduced effector function, unless otherwise specified. The Fc domain may be the portion of a polypeptide which corresponds to the Fc domain of native Ig, i.e., as formed by the dimeric association of the respective Fc domains of its two heavy chains. A native Fc domain forms a homodimer with another Fc domain.

In some aspects, the “Fc region” refers to the portion of a single Ig heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an Ig constant region, depending on the Ig isotype can include the CH2, CH3, and CH4 domains, as well as the hinge region. Fusion proteins comprising an Fc region of an Ig bestow several desirable properties on a fusion protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

Fc regions useful in the present disclosure encompass molecules that can specifically bind an FcR, including whole IgG, the Fc fragment of IgG, and other fragments that include the complete binding region of an FcR. The region of the Fc portion of IgG that binds to, e.g., the FcRn receptor has been described based on X-ray crystallography (Burmeister et al. 1994, Nature 372:379). The major contact area of the Fc with the FcRn is near the junction of the CH2 and CH3 domains.

Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 10⁸ M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g. serum albumin, milk casein), etc., can be optimized by a skilled artisan using routine techniques.

In certain aspects, a fusion protein of the disclosure comprises one or more truncated Fc regions that are nonetheless sufficient to confer FcR binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain. Thus, an Fc region of the disclosure can comprise or consist of an FcRn binding portion.

FcR binding portions can be derived from heavy chains of any isotype, including IgG1, IgG2, IgG3 and IgG4. In some aspect, an FcR binding portion from an antibody of the human isotype IgG1 is used. In some aspect, an FcR binding portion from an antibody of the human isotype IgG2 is used. In some aspect, an FcR binding portion from an antibody of the human isotype IgG3 is used. In some aspects, an FcR binding portion from an antibody of the human isotype IgG4 is used.

In another aspect, the “Fc region” includes an amino acid sequence of an Fc domain or derived from an Fc domain. In certain aspects, an Fc region comprises at least one of: a hinge (e.g., upper, middle, and/or lower hinge region) domain (about amino acids 216-230 of an antibody Fc region according to EU numbering), a CH2 domain (about amino acids 231-340 of an antibody Fc region according to EU numbering), a CH3 domain (about amino acids 341-438 of an antibody Fc region according to EU numbering), a CH4 domain, or a variant, portion, or fragment thereof. In other aspects, an Fc region comprises a complete Fc domain (i.e., a hinge domain, a CH2 domain, and a CH3 domain). In some aspects, an Fc region comprises, consists essentially of, or consists of a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), a CH2 domain (or a portion thereof) fused to both a hinge domain (or a portion thereof) and a CH3 domain (or a portion thereof). In still other aspects, an Fc region lacks at least a portion of a CH2 domain (e.g., all or part of a CH2 domain). In a particular aspect, an Fc region comprises or consists of amino acids corresponding to EU numbers 221 to 447.

In some aspects, an Fc region of the polypeptide is derived from a human Ig. It is understood, however, that an Fc region can be derived from an Ig of another mammalian species, including for example, a rodent (e.g. a mouse, rat, rabbit, or guinea pig) or non-human primate (e.g. chimpanzee, macaque) species. Moreover, the polypeptide of the Fc domains or portions thereof can be derived from any Ig class, including IgM, IgG, IgD, IgA and IgE, and any Ig isotype, including IgG1, IgG2, IgG3 and IgG4. In another aspect, the human isotype IgG1 is used.

In certain aspects, the Fc polypeptide may confer a change in at least one effector function imparted by an Fc region comprising the wild-type Fc domain (e.g., an improvement or reduction in the ability of the Fc region to bind to Fc receptors (e.g., improvement or reduction in binding to FcγRI, FcγRII, or FcγRIII), complement proteins (e.g. C1q), or other Fc binding partners (e.g. DC-SIGN), or to alter, trigger, enhance, or reduce antibody-dependent cytotoxicity (ADCC), phagocytosis, or complement-dependent cytotoxicity (CDCC)). In certain aspects, the Fc polypeptide may have reduced ADCC. In other aspects, the Fc variant provides an engineered cysteine residue.

The Fc regions of the disclosure can employ art-recognized Fc variants which are known to impart a change (e.g., an enhancement or reduction) in effector function and/or FcR or FcRn binding. Specifically, a binding molecule of the disclosure can include, for example, a change (e.g., a substitution) at one or more of the amino acid positions disclosed in International PCT Publications WO88/07089, WO96/14339, WO98/05787, WO98/23289, WO99/51642, WO99/58572, WO00/09560, WO00/32767, WO00/42072, WO02/44215, WO02/060919, WO03/074569, WO04/016750, WO04/029207, WO04/035752, WO04/063351, WO04/074455, WO04/099249, WO05/040217, WO04/044859, WO05/070963, WO05/077981, WO05/092925, WO05/123780, WO06/019447, WO06/047350, and WO06/085967; US Patent Publication Nos. US2007/0231329, US2007/0231329, US2007/0237765, US2007/0237766, US2007/0237767, US2007/0243188, US2007/0248603, US2007/0286859, US2008/0057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091; each of which is incorporated by reference herein in its entirety. In some aspects, the specific change (e.g., the specific substitution of one or more amino acids disclosed in the art) can be made at one or more of the disclosed amino acid positions. In another aspect, a different change at one or more of the disclosed amino acid positions (e.g., the different substitution of one or more amino acid position disclosed in the art) can be made.

The Fc region can be modified according to well recognized procedures such as site directed mutagenesis and the like to yield modified Fc fragments or portions thereof that will be bound by FcγRIIB and/or DC-SIGN. Such modifications include modifications remote from the FcγRIIB and/or DC-SIGN contact sites as well as modifications within the contact sites that preserve or alter binding to the FcγRIIB and/or DC-SIGN. Mutations can be introduced singly into Fc giving rise to more than one hundred Fc regions distinct from the native Fc. Additionally, combinations of two, three, or more of these individual mutations can be introduced together, giving rise to hundreds more Fc regions. Moreover, one of the Fc region of a construct of the disclosure can be mutated and the other Fc region of the construct not mutated at all, or they both can be mutated but with different mutations.

Certain of the above mutations can confer new functionality upon the Fc region or FcRn binding partner. For example, one aspect incorporates N297A, removing a highly conserved N-glycosylation site. This mutation enhances circulating half-life of the Fc region, and renders the Fc region incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for FcRn can be increased beyond that of wild type in some instances. This increased affinity can reflect an increased “on” rate, a decreased “off” rate, or both an increased “on” rate and a decreased “off” rate. Examples of mutations believed to impart an increased affinity for FcRn include, but are not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity can arise from mutations of this region, as for example by replacing amino acids 233-236 of human IgG1 “ELLG” to the corresponding sequence from IgG2 “PVA” (with one amino acid deletion). It has been shown that FcγRI, FcγRII, and FcγRIII, which mediate various effector functions, will not bind to IgG1 when such mutations have been introduced. Ward and Ghetie 1995, Therapeutic Immunology 2:77 and Armour et al. 1999, Eur. J. Immunol. 29:2613.

In some aspects, the Fc domain or a portion thereof is a polypeptide including SEQ ID NO: 3 of U.S. Pat. No. 5,739,277 and optionally further including a sequence selected from SEQ ID NOs: 11, 1, 2, and 31 of U.S. Pat. No. 5,739,277.

In certain aspects, the Fc domain or a portion thereof is hemi-glycosylated, wherein a fusion protein comprises at least two Fc regions, and wherein at least one Fc region is glycosylated (e.g., a glycosylated CH2 region) and at least one Fc region is aglycosylated (e.g., an aglycosylated CH2 region). In some aspects, a linker can be interposed between the glycosylated and aglycosylated Fc regions. In another aspect, the Fc region is fully glycosylated, i.e., all of the Fc regions are glycosylated. In other aspects, the Fc region can be aglycosylated, i.e., none of the Fc moieties are glycosylated.

In certain aspects, a fusion protein of the disclosure comprises an amino acid substitution to an Fc domain or a portion thereof (e.g., Fc variants), which alters the antigen-independent effector functions of Fc domain, in particular the circulating half-life of the protein.

Such proteins exhibit either increased or decreased binding to FcR when compared to proteins lacking these substitutions and, therefore, have an increased or decreased half-life in serum, respectively. Fc variants with improved affinity for FcR are anticipated to have longer serum half-lives, and such molecules have useful applications in methods of treating mammals where long half-life of the administered polypeptide is desired, e.g., to treat a chronic disease or disorder (see, e.g., U.S. Pat. Nos. 7,348,004, 7,404,956, and 7,862,820). In contrast, Fc variants with decreased FcR binding affinity are expected to have shorter half-lives, and such molecules are also useful, for example, for administration to a mammal where a shortened circulation time can be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization the brain, kidney, and/or liver is desired. In one exemplary aspect, the fusion protein of the disclosure exhibits reduced transport across the epithelium of kidney glomeruli from the vasculature. In another aspect, the fusion protein of the disclosure exhibits reduced transport across the blood brain barrier (BBB) from the brain, into the vascular space. In some aspects, a protein with altered FcR binding comprises at least one Fc region (e.g., one or two Fc regions) having one or more amino acid substitutions within the “FcR binding loop” of an Ig constant region. The FcR binding loop is, in some aspects, comprised of amino acid residues 280-299 (according to EU numbering) of a wild-type, full-length, Fc region. In other aspects, an Ig constant region or a portion thereof in a chimeric protein of the disclosure having altered FcR binding affinity comprises at least one Fc region having one or more amino acid substitutions within the 15 \acute{Å} FcR “contact zone.” Exemplary amino acid substitutions which altered FcR binding activity are disclosed in International PCT Publication No. WO 05/047327 and U.S. Publication No. US2012003210 (A1), each of which is incorporated by reference herein in its entirety. An Fc region used in the disclosure can also comprise an art recognized amino acid substitution which alters the glycosylation of the fusion protein. For example, the Fc region of the fusion protein linked to an IL-10 polypeptide disclosed herein can comprise an Fc region having a mutation leading to altered glycosylation (e.g., N- or O-linked glycosylation) or can comprise an altered effector function.

In some aspects, a fusion protein of the disclosure can comprise a genetically fused Fc region (i.e., scFc region) having two or more of its constituent Ig constant region or a portion thereof independently selected from the Ig constant region or a portion thereof described herein. In some aspects, the Fc regions of a dimeric Fc region are the same. In another aspect, at least two of the Fc regions are different. For example, the Fc regions of the proteins of the disclosure comprise the same number of amino acid residues or they can differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other aspects, the Fc regions of the protein of the disclosure can differ in sequence at one or more amino acid positions. For example, at least two of the Fc regions can differ at about 5 amino acid positions (e.g., 1, 2, 3, 4, or 5 amino acid positions), about 10 positions, about 15 positions, about 20 positions, about 30 positions, about 40 positions, or about 50 positions).

A variety of the Fc region gene sequences (e.g., human Fc gene sequences) are available in the form of publicly accessible deposits. Fc sequences can be selected having a particular effector function (or lacking a particular effector function) or with a particular modification to reduce immunogenicity or ADCC. Many sequences of antibodies and antibody-encoding genes have been published and suitable Fc region sequences can be derived from these sequences using art recognized techniques. The genetic material obtained using any of the foregoing methods can then be altered or synthesized to obtain chimeric proteins used in the methods of the present disclosure. It will further be appreciated that the scope of this disclosure encompasses alleles, variants and mutations of constant region DNA sequences.

In some aspects, the Fc polypeptide comprises one or more modification that results in a reduced antibody-dependent cellular cytotoxicity (ADCC). In some aspects, the Fc polypeptide comprises an IgG1 Fc region, comprising one or more substitution selected from L234A, L235E, G237A, P238K, according to EU numbering, and any combination thereof. In some aspects, the Fc polypeptide comprises an IgG1 Fc region, comprising a L234A substitution. In some aspects, the Fc polypeptide comprises an IgG1 Fc region, comprising a L235E substitution. In some aspects, the Fc polypeptide comprises an IgG1 Fc region, comprising a G237A substitution. In some aspects, the Fc polypeptide comprises an IgG1 Fc region, comprising a P238K substitution. In some aspects, the Fc polypeptide comprises an IgG1 Fc region, comprising L234A, L235E, and G237A substitutions. In some aspects, the Fc polypeptide comprises an IgG1 Fc region, comprising a terminal K residue. In some aspects, the Fc polypeptide comprises an IgG1 Fc region, lacking a terminal K residue. In some aspects, the Fc polypeptide comprises an IgG1 Fc region, comprising a terminal G residue. In some aspects, the Fc polypeptide comprises an IgG1 Fc region, comprising a cysteine bridge, e.g., a cysteine bridge variant. In some aspects, the cysteine bridge variant comprises a N-terminal VEPKSC (SEQ ID NO: 13). In certain aspects, the Fc polypeptide comprises an IgG1.3f Fc region.

In some aspects, the Fc polypeptide comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 4-12 (Table 2). In some aspects, the Fc polypeptide comprises an amino acid sequence selected from SEQ ID NOs: 4-12. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 4. In some aspects, the Fc polypeptide comprises the amino acid sequence set forth in SEQ ID NO: 4. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 5. In some aspects, the Fc polypeptide comprises the amino acid sequence set forth in SEQ ID NO: 5. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 6. In some aspects, the Fc polypeptide comprises the amino acid sequence set forth in SEQ ID NO: 6. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 7. In some aspects, the Fc polypeptide comprises the amino acid sequence set forth in SEQ ID NO: 7. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 8. In some aspects, the Fc polypeptide comprises the amino acid sequence set forth in SEQ ID NO: 8. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 9. In some aspects, the Fc polypeptide comprises the amino acid sequence set forth in SEQ ID NO: 9. In some aspects, the Fc polypeptide comprises an amino acid sequence at least about 85%, at least about 90%, at least about 95%, at least about 96%, at least about 97%, at least about 98%, or at least about 99% sequence identity to the amino acid sequence set forth in SEQ ID NO: 10. In some aspects, the Fc polypeptide comprises the amino acid sequence set forth in SEQ ID NO: 10. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 11. In some aspects, the Fc polypeptide comprises the amino acid sequence set forth in SEQ ID NO: 11. In some aspects, the Fc polypeptide comprises an amino acid sequence having at least about 85%, at least about 90%, at least about 95%, at least about 96%, at least about 97%, at least about 98%, or at least about 99% sequence identity to the amino acid sequence set forth in SEQ ID NO: 12. In some aspects, the Fc polypeptide comprises the amino acid sequence set forth in SEQ ID NO: 12.

TABLE 2
Exemplery Fc Polypeptide Sequences.

| SEQ | | |
|---|---|---|
| ID | | |
| NO | Description | Sequence |
| 4 | Fc(Ig1.3f) | DKTHTCPPCPAPEAEGAPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PG |
| 5 | Fc IgGlf | DKTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 6 | Fc | DKTHTCPPCPAPELLGGPSVFLFPPKPKDTLMISRTPEVTCVVVDVSHEDPEVKFN |
| | IgG1a/z | WYVDGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 7 | Fc IgG1 | DKTHTCPPCPAPEAEGAPSVFLFPPKPKDTLMISRTPEVTCVVVDVSHEDPEVKFN |
| | (L234A, | WYVDGVEVHNAKTKPREEQYNSTYRVVSVLTVLHQDWLNGKEYKCKVSNKALPAPI |
| | L235E, G23 | EKTISKAKGQPREPQVYTLPPSREEMTKNQVSLTCLVKGFYPSDIAVEWESNGQPE |
| | 7A) | NNYKTTPPVLDSDGSFFLYSKLTVDKSRWQQGNVFSCSVMHEALHNHYTQKSLSLS |
| | | PGK |
| 8 | Fc IgG1 | DKTHTCPPCPAPLLEGGKSVFLFPPKPKDTLMISRTPEVTCVVVDVSHEDPEVKFN |
| | (P238K) | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 9 | Fc IgG1f | DKTHTCPPCPAPELLGGPSVFLFPPKPKDTLMISRTPEVTCVVVDVSHEDPEVKFN |
| | sans | WYVDGVEVHNAKTKPREEQYNSTYRVVSVLTVLHQDWLNGKEYKCKVSNKALPAPI |
| | Terminal | EKTISKAKGQPREPQVYTLPPSREEMTKNQVSLTCLVKGFYPSDIAVEWESNGQPE |
| | K | NNYKTTPPVLDSDGSFFLYSKLTVDKSRWQQGNVFSCSVMHEALHNHYTQKSLSLS |
| | | PG |
| 10 | IgG1.3f | DKTHTCPPCPAPEAEGAPSVFLFPPKPKDTLMISRTPEVTCVVVDVSHEDPEVKFN |
| | sans | WYVDGVEVHNAKTKPREEQYNSTYRVVSVLTVLHQDWLNGKEYKCKVSNKALPAPI |
| | Terminal | EKTISKAKGQPREPQVYTLPPSREEMTKNQVSLTCLVKGFYPSDIAVEWESNGQPE |
| | K | NNYKTTPPVLDSDGSFFLYSKLTVDKSRWQQGNVFSCSVMHEALHNHYTQKSLSLS |
| | | PG |
| 11 | Fc-IgG1 | DKTHTCPPCPAPLLEGGKSVFLFPPKPKDTLMISRTPEVTCVVVDVSHEDPEVKFN |
| | P238K | WYVDGVEVHNAKTKPREEQYNSTYRVVSVLTVLHQDWLNGKEYKCKVSNKALPAPI |
| | sans | EKTISKAKGQPREPQVYTLPPSREEMTKNQVSLTCLVKGFYPSDIAVEWESNGQPE |
| | Terminal | NNYKTTPPVLDSDGSFFLYSKLTVDKSRWQQGNVFSCSVMHEALHNHYTQKSLSLS |
| | K | PG |
| 12 | Fc - 3 | VEPKSCDKTHTCPPCPAPEAEGAPSVFLFPPKPKDTLMISRTPEVTCVVVDVSHED |
| | Cysteine | PEVKFNWYVDGVEVHNAKTKPREEQYNSTYRVVSVLTVLHQDWLNGKEYKCKVSNK |
| | bridge | ALPAPIEKTISKAKGQPREPQVYTLPPSREEMTKNQVSLTCLVKGFYPSDIAVEWE |
| | variant | SNGQPENNYKTTPPVLDSDGSFFLYSKLTVDKSRWQQGNVFSCSVMHEALHNHYTQ |
| | | KSLSLSPGK |

### II.C. Linkers

In certain aspects, the IL-10 polypeptide is linked to the Fc polypeptide by a linker. Any linker known in the art can be used in the fusion proteins disclosed herein. In some aspects, the linker comprises a chemical linker. In some aspects, the linker comprises a covalent bond. In some aspects, the linker comprises a peptide bond. In some aspects, the linker comprises one or more amino acids. In some aspect, the linker comprises a peptide linker.

The linker can be any length. In some aspects, the linker comprises at least about 1 to at least about 100 amino acids, at least about 1 to at least about 75 amino acids, at least about 1 to at least about 50 amino acids, at least about 1 to at least about 40 amino acids, at least about 1 to at least about 30 amino acids, at least about 1 to at least about 25 amino acids, at least about 1 to at least about 20 amino acids, at least about 1 to at least about 15 amino acids, at least about 1 to at least about 10 amino acids, or at least about 1 to at least about 5 amino acids. In some aspects, the linker comprises at least about 5 to at least about 100 amino acids, at least about 5 to at least about 75 amino acids, at least about 5 to at least about 50 amino acids, at least about 5 to at least about 40 amino acids, at least about 5 to at least about 30 amino acids, at least about 5 to at least about 25 amino acids, at least about 5 to at least about 20 amino acids, at least about 5 to at least about 15 amino acids, or at least about 5 to at least about 10 amino acids. In some aspects, the linker comprises at least about 10 to at least about 100 amino acids, at least about 10 to at least about 75 amino acids, at least about 10 to at least about 50 amino acids, at least about 10 to at least about 40 amino acids, at least about 10 to at least about 30 amino acids, at least about 10 to at least about 25 amino acids, at least about 10 to at least about 20 amino acids, or at least about 10 to at least about 15 amino acids. In some aspects, the linker comprises at least about 5 to at least about 25 amino acids. In some aspects, the linker comprises at least about 10 to at least about 25 amino acids. In some aspects, the linker comprises at least about 10 to at least about 20 amino acids. In some aspects, the linker comprises at least about 15 to at least about 25 amino acids.

In some aspects, the linker comprises at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 21 amino acids, at least about 22 amino acids, at least about 23 amino acids, at least about 24 amino acids, at least about 25 amino acids, or at least about 30 amino acids. In some aspects, the linker comprises about 4 amino acids. In some aspects, the linker comprises about 5 amino acids. In some aspects, the linker comprises about 8 amino acids. In some aspects, the linker comprises about 10 amino acids. In some aspects, the linker comprises about 11 amino acids. In some aspects, the linker comprises about 15 amino acids. In some aspects, the linker comprises about 20 amino acids. In some aspects, the linker comprises about 21 amino acids. In some aspects, the linker comprises about 22 amino acids.

Various peptide linkers are known in the art and can be used in the fusion proteins of the present disclosure. In some aspects, the linker comprises a Glycine-Serine linker, e.g., a linker comprising at least one Glycine residue and at least one Serine residue. Any combination of Glycine and Serine residues can be used. In some aspects, the linker comprises G-S. In some aspects, the linker comprises G-G-S. In some aspects, the linker comprises GGGS (SEQ ID NO: 38). In some aspects, the linker comprises GGGGS (SEQ ID NO: 39). In some aspects, the linker comprises GGGGGS (SEQ ID NO: 40). In some aspects, the linker comprises at least one GGGS (SEQ ID NO: 38) motif. In some aspects, the linker comprises at least two GGGS (SEQ ID NO: 38) motifs. In some aspects, the linker comprises at least three GGGS (SEQ ID NO: 38) motifs. In some aspects, the linker comprises at least four GGGS (SEQ ID NO: 38) motifs.

In some aspects, the linker comprises at least one GGGGS (SEQ ID NO: 39) motif. In some aspects, the linker comprises at least two GGGGS (SEQ ID NO: 39) motifs. In some aspects, the linker comprises at least three GGGGS (SEQ ID NO: 39) motifs. In some aspects, the linker comprises at least four GGGGS (SEQ ID NO: 39) motifs.

In certain aspects, the linker comprises an amino acid sequence selected from SEQ ID NOs: 41-45 (Table 3). In some aspects, the linker comprises the amino acid sequence set forth in SEQ ID NO: 41. In some aspects, the linker comprises the amino acid sequence set forth in SEQ ID NO: 42. In some aspects, the linker comprises the amino acid sequence set forth in SEQ ID NO: 43. In some aspects, the linker comprises the amino acid sequence set forth in SEQ ID NO: 44. In some aspects, the linker comprises the amino acid sequence set forth in SEQ ID NO: 45.

TABLE 3
Exemplery Linker Sequences.

| SEQ | |
|---|---|
| ID | |
| NO | Sequence |
| 38 | GGGS |
| 39 | GGGGS |
| 40 | GGGGGS |
| 41 | GGGGSSGGGGSGGGGSGGGGS |
| 42 | GGGGSGGGGSGGGGSGGGGS |
| 43 | GGGGSGGGGSGGGGS |
| 44 | GGGGSGGGGS |
| 45 | GGGGSSGGGGS |

In some aspects, the linker is a cleavable linker. In some aspects, the linker comprises an enzymatic cleavage cite. In some aspects, the linker is capable of being cleaved by one or more enzyme that is present at a target tissue. In some aspects, cleavage of the linker results in the release of the IL-10 polypeptide from the Fc polypeptide. Any cleavable linker known in the art can be used alone or in conjunction with one or more other linker disclosed herein.

### II.D. IL-10 Fusion Proteins

Certain aspects of the present disclosure are directed to fusion proteins comprising an IL-10 polypeptide disclosed herein and an Fc polypeptide disclosed herein. In some aspects, the fusion protein has an IL-10 function. In certain aspects, the fusion protein is capable of enhancing interferon gamma (IFNγ) levels. In certain aspects, the fusion protein is capable of inducing IFNγ, e.g., in human CD8⁺ T cells. In some aspects, the fusion protein is capable of inducing cell-mediated cytotoxicity of human NK cells. In some aspects, the fusion protein is capable of forming a dimer, e.g., a homodimer with a second fusion protein or a heterodimer with a hIL-10 protein. In some aspects, the fusion protein is capable of binding to an IL-10 receptor. In some aspects, the fusion protein is capable of activating Jak1. In some aspects, the fusion protein is capable of activating Tyk2. In some aspects, the fusion protein is capable of activating STAT1. In some aspects, the fusion protein is capable of activating STAT3. In some aspects, the fusion protein is capable of activating STATS. In some aspects, the fusion protein is capable of eliciting an anti-inflammatory response. In some aspects, the fusion protein is capable of eliciting a pro-inflammatory response.

Any orientation of the IL-10 polypeptide and the Fc polypeptide is contemplated by the present disclosure. Accordingly, in some aspects, the N-terminus of the IL-10 polypeptide is linked (directly or indirectly) to the C-terminus of Fc polypeptide, e.g., Fc-IL-10. In some aspects, the N-terminus of IL-10 polypeptide is linked directly to the C-terminus of the Fc polypeptide, e.g., without a peptide linker. In some aspects, the N-terminus of IL-10 polypeptide is linked to the C-terminus of the Fc polypeptide by one or more amino acids. In some aspects, the N-terminus of IL-10 polypeptide is linked to the C-terminus of the Fc polypeptide by a peptide linker, e.g., by any peptide linker disclosed herein.

In some aspects, the fusion protein comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 14-32 (Table 4).

In some aspects, the fusion protein comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 14. In some aspects, the fusion protein comprises an amino acid sequence having at least about 90% sequence identity to the amino acid sequence set forth in SEQ ID NO: 14. In some aspects, the fusion protein comprises an amino acid sequence having at least about 95% sequence identity to the amino acid sequence set forth in SEQ ID NO: 14. In some aspects, the fusion protein comprises an amino acid sequence having at least about 96% sequence identity to the amino acid sequence set forth in SEQ ID NO: 14. In some aspects, the fusion protein comprises an amino acid sequence having at least about 97% sequence identity to the amino acid sequence set forth in SEQ ID NO: 14. In some aspects, the fusion protein comprises an amino acid sequence having at least about 98% sequence identity to the amino acid sequence set forth in SEQ ID NO: 14. In some aspects, the fusion protein comprises an amino acid sequence having at least about 99% sequence identity to the amino acid sequence set forth in SEQ ID NO: 14. In some aspects, the fusion protein comprises the amino acid sequence set forth in SEQ ID NO: 14.

In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 15. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 16. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 17. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 18. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 19. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 20. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 21. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 22. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 23. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 24. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 25. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 26. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 27. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 28. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 29. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 30. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 31. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 32.

TABLE 4
Exemplary Fusion Protein Amino Acid Sequences.

| SEQ | |
|---|---|
| ID | |
| NO | Sequence |
| 14 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSGGGGSGGGGSGGGGSSPGQGTQSE |
| | NSCTHFPGNLPNMLRDLRDAFSRVKTFFQMKDQLDNLLLKESLLEDFKGYLGCQALSEMIQFYL |
| | EEVMPQAENQDPDIKAHVNSLGENLKTLRLRLRRCHRFLPCENKSKAVEQVKNAFNKLQEKGIY |
| | KAMSEFDIFINYIEAYMTMKIRN |
| 15 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSSGGGGSGGGGSGGGGSSPGQGTQS |
| | ENSCTHFPGNLPNMLRDLRDAFSRVKTFFQMKDQLDNLLLKESLLEDFKGYLGCQALSEMIQFY |
| | LEEVMPQAENQDPDIKAHVNSLGENLKTLRLRLRRCHRFLPCENKSKAVEQVKNAFNKLQEKGI |
| | YKAMSEFDIFINYIEAYMTMKIRN |
| 16 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCENKSKAVEQVKNAFNKLQEKGIYK |
| | AMSEFDIFINYIEAYMTMKIRN |
| 17 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCENKSKAVEQVKNAFNKLQEKGIYK |
| | AMSEFDIFINYIEAYMTMKIRN |
| 18 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPSPGQGTQSENSCTHFPGNLPNMLRDLRDAFS |
| | RVKTFFQMKDQLDNLLLKESLLEDFKGYLGCQALSEMIQFYLEEVMPQAENQDPDIKAHVNSLG |
| | ENLKTLRLRLRRCHRFLPCENKSKAVEQVKNAFNKLQEKGIYKAMSEFDIFINYIEAYMTMKIR |
| | N |
| 19 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSPGQGTQSENSCTHFPGNLPNMLRD |
| | LRDAFSRVKTFFQMKDQLDNLLLKESLLEDFKGYLGCQALSEMIQFYLEEVMPQAENQDPDIKA |
| | HVNSLGENLKTLRLRLRRCHRFLPCENKSKAVEQVKNAFNKLQEKGIYKAMSEFDIFINYIEAY |
| | MTMKIRN |
| 20 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSSPGQGTQSENSCTHFPGNLP |
| | NMLRDLRDAFSRVKTFFQMKDQLDNLLLKESLLEDFKGYLGCQALSEMIQFYLEEVMPQAENQD |
| | PDIKAHVNSLGENLKTLRLRLRRCHRFLPCENKSKAVEQVKNAFNKLQEKGIYKAMSEFDIFIN |
| | YIEAYMTMKIRN |
| 21 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSPGQGTQSENSCTHFPGNLPNMLRD |
| | LRDAFSRVKTFFQMKDQLDNLLLKESLLEDFKGYLGCQALSEMIQFYLEEVMPQAENQDPDIKA |
| | HVNSLGENLKTLRLRLRRCHRFLPCENKSKAVEQVKNAFNKLQEKGIYKAMSEFDIFINYIEAY |
| | MTMKIRN |
| 22 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSSPGQGTQSENSCTHFPGNLP |
| | NMLRDLRDAFSRVKTFFQMKDQLDNLLLKESLLEDFKGYLGCQALSEMIQFYLEEVMPQAENQD |
| | PDIKAHVNSLGENLKTLRLRLRRCHRFLPCENKSKAVEQVKNAFNKLQEKGIYKAMSEFDIFIN |
| | YIEAYMTMKIRN |
| 23 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSSPGQGTQSENSCTHF |
| | PGNLPNMLRDLRDAFSRVKTFFQMKDQLDNLLLKESLLEDFKGYLGCQALSEMIQFYLEEVMPQ |
| | AENQDPDIKAHVNSLGENLKTLRLRLRRCHRFLPCENKSKAVEQVKNAFNKLQEKGIYKAMSEF |
| | DIFINYIEAYMTMKIRN |
| 24 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSGGGGSSPGQGTQSENSCTHFPGNL |
| | PNMLRDLRDAFSRVKTFFQMKDQLDNLLLKESLLEDFKGYLGCQALSEMIQFYLEEVMPQAENQ |
| | DPDIKAHVNSLGENLKTLRLRLRRCHRFLPCENKSKAVEQVKNAFNKLQEKGIYKAMSEFDIFI |
| | NYIEAYMTMKIRN |
| 25 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSGGGGSGGGGSSPGQGTQSENSCTH |
| | FPGNLPNMLRDLRDAFSRVKTFFQMKDQLDNLLLKESLLEDFKGYLGCQALSEMIQFYLEEVMP |
| | QAENQDPDIKAHVNSLGENLKTLRLRLRRCHRFLPCENKSKAVEQVKNAFNKLQEKGIYKAMSE |
| | FDIFINYIEAYMTMKIRN |
| 26 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSSGGGGSGGGGSGGGGSSPGQGTQSE |
| | NSCTHFPGNLPNMLRDLRDAFSRVKTFFQMKDQLDNLLLKESLLEDFKGYLGCQALSEMIQFYL |
| | EEVMPQAENQDPDIKAHVNSLGENLKTLRLRLRRCHRFLPCENKSKAVEQVKNAFNKLQEKGIY |
| | KAMSEFDIFINYIEAYMTMKIRN |
| 27 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSED |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCENKSKAVEQVKNAFNKLQEKGIYK |
| | AMSEFDIFINYIEAYMTMKIRN |
| 28 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVDSLGENLKTLRLRLRRCHRFLPCENKSKAVEQVKNAFNKLQEKGIYK |
| | AMSEFDIFINYIEAYMTMKIRN |
| 29 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCENKSKAVEQVKDAFNKLQEKGIYK |
| | AMSEFDIFINYIEAYMTMKIRN |
| 30 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCEDKSKAVEQVKNAFNKLQEKGIYK |
| | AMSEFDIFINYIEAYMTMKIRN |
| 31 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAEDQDPDIKAHVNSLGENLKTLRLRLRRCHRFLPCENKSKAVEQVKNAFNKLQEKGIYK |
| | AMSEFDIFINYIEAYMTMKIRN |
| 32 | DKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSGGGGSSPGQGTQSEN |
| | SCTHFPGNLPNMLRDLRDAFSRVKTFFQMKDQLDNLLLKESLLEDFKGYLGCQALSEMIQFYLE |
| | EVMPQAENQDPDIKAHVNSLGENLKTLRLRLRRCHRFLPCENKSKAVEQVKNAFNKLQEKGIYK |
| | AMSEFDIFINYIEAYMTMKIR |
| 33 | SPGQGTQSENSCTHFPGNLPNMLRDLRDAFSRVKTFFQMKDQLDNLLLKESLLEDFKGYLGCQA |
| | LSEMIQFYLEEVMPQAENQDPDIKAHVNSLGENLKTLRLRLRRCHRFLPCENKSKAVEQVKNAF |
| | NKLQEKGIYKAMSEFDIFINYIEAYMTMKIRNGGGGSVEPKSCDKTHTCPPCPAPEAEGAPSVF |
| | LFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEALH |
| | NHYTQKSLSLSPG |
| 34 | SPGQGTQSENSCTHFPGNLPNMLRDLRDAFSRVKTFFQMKDQLDNLLLKESLLEDFKGYLGCQA |
| | LSEMIQFYLEEVMPQAENQDPDIKAHVNSLGENLKTLRLRLRRCHRFLPCENKSKAVEQVKNAF |
| | NKLQEKGIYKAMSEFDIFINYIEAYMTMKIRNGGGGSGGGGSVEPKSCDKTHTCPPCPAPEAEG |
| | APSVFLFPPKPKDTLMISRTPEVTCVVVDVSHEDPEVKFNWYVDGVEVHNAKTKPREEYQNSTY |
| | RVVSVLTVLHQDWLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSRWQQGNVFSCSVM |
| | HEALHNHYTQKSLSLSPG |
| 35 | SPGQGTQSENSCTHFPGNLPNMLRDLRDAFSRVKTFFQMKDQLDNLLLKESLLEDFKGYLGCQA |
| | LSEMIQFYLEEVMPQAENQDPDIKAHVNSLGENLKTLRLRLRRCHRFLPCENKSKAVEQVKNAF |
| | NKLQEKGIYKAMSEFDIFINYIEAYMTMKIRNGGGGSGGGGSGGGGSVEPKSCDKTHTCPPCPA |
| | PEAEGAPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTVDKSRWQQGNVF |
| | SCSVMHEALHNHYTQKSLSLSPG |
| 36 | SPGQGTQSENSCTHFPGNLPNMLRDLRDAFSRVKTFFQMKDQLDNLLLKESLLEDFKGYLGCQA |
| | LSEMIQFYLEEVMPQAENQDPDIKAHVNSLGENLKTLRLRLRRCHRFLPCENKSKAVEQVKNAF |
| | NKLQEKGIYKAMSEFDIFINYIEAYMTMKIRNGGGGSGGGGSGGGGSGGGGSVEPKSCDKTHTC |
| | PPCPAPEAEGAPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFFLYSKLTVDKSRWQ |
| | QGNVFSCSVMHEALHNHYTQKSLSLSPG |

In some aspects, the fusion protein comprises an amino acid sequence selected from SEQ ID NOs: 14-32 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 14 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 15 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 16 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 17 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 18 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 19 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 20 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 21 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 22 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 23 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 24 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 25 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 26 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 27 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 28 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 29 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 30 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 31 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 32 with 10 or fewer, 9 or fewer, 8 or fewer, 7 or fewer, 6 or fewer, 5 or fewer, 4 or fewer, 3 or fewer, 2 or fewer, substitutions, insertions, or deletions.

In some aspects, the fusion protein comprises the amino acid sequence set forth in SEQ ID NO: 14. In some aspects, the fusion protein comprises the amino acid sequence set forth in SEQ ID NO: 15. In some aspects, the fusion protein comprises the amino acid sequence set forth in SEQ ID NO: 16. In some aspects, the fusion protein comprises the amino acid sequence set forth in SEQ ID NO: 17. In some aspects, the fusion protein comprises the amino acid sequence set forth in SEQ ID NO: 18. In some aspects, the fusion protein comprises the amino acid sequence set forth in SEQ ID NO: 19. In some aspects, the fusion protein comprises the amino acid sequence set forth in SEQ ID NO: 20. In some aspects, the fusion protein comprises the amino acid sequence set forth in SEQ ID NO: 21. In some aspects, the fusion protein comprises the amino acid sequence set forth in SEQ ID NO: 22. In some aspects, the fusion protein comprises the amino acid sequence set forth in SEQ ID NO: 23. In some aspects, the fusion protein comprises the amino acid sequence set forth in SEQ ID NO: 24. In some aspects, the fusion protein comprises the amino acid sequence set forth in SEQ ID NO: 25. In some aspects, the fusion protein comprises the amino acid sequence set forth in SEQ ID NO: 26. In some aspects, the fusion protein comprises the amino acid sequence set forth in SEQ ID NO: 27. In some aspects, the fusion protein comprises the amino acid sequence set forth in SEQ ID NO: 28. In some aspects, the fusion protein comprises the amino acid sequence set forth in SEQ ID NO: 29. In some aspects, the fusion protein comprises the amino acid sequence set forth in SEQ ID NO: 30. In some aspects, the fusion protein comprises the amino acid sequence set forth in SEQ ID NO: 31. In some aspects, the fusion protein comprises the amino acid sequence set forth in SEQ ID NO: 32.

In some aspects, the fusion protein comprises an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 33-36 (Table 4). In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 33. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 34. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 35. In some aspects, the fusion protein comprises an amino acid sequence having at least about 90%, at least about 95%, at least about 96%, at least about 97%, at least about 98%, or at least about 99% sequence identity to the amino acid sequence set forth in SEQ ID NO: 36.

In some aspects, the fusion protein comprises an amino acid sequence selected from SEQ ID NOs: 33-36 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 33 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 34 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 35 with 10 or fewer, 9 or fewer, 8 or fewer, 7 or fewer, 6 or fewer, 5 or fewer, 4 or fewer, 3 or fewer, 2 or fewer, substitutions, insertions, or deletions. In some aspects, the fusion protein comprises the amino acid sequence set forth in SEQ ID NO: 36 with 10 or fewer, 9 or fewer, 8 or fewer, 7 or fewer, 6 or fewer, 5 or fewer, 4 or fewer, 3 or fewer, 2 or fewer, substitutions, insertions, or deletions.

In some aspects, the fusion protein comprises the amino acid sequence set forth in SEQ ID NO: 33. In some aspects, the fusion protein comprises the amino acid sequence set forth in SEQ ID NO: 34. In some aspects, the fusion protein comprises the amino acid sequence set forth in SEQ ID NO: 35. In some aspects, the fusion protein comprises the amino acid sequence set forth in SEQ ID NO: 36.

### II.E. IL-10 Fusion Protein Dimers

In certain aspects, the fusion protein comprises an IL-10 dimer. In some aspects, the IL-10 dimer comprises a first polypeptide and a second polypeptide, wherein the first polypeptide comprises any IL-10 fusion protein described herein, and wherein the second polypeptide comprises a second Fc polypeptide. In certain aspects, the first Fc polypeptide and the second Fc polypeptide are linked or associated by a covalent bond. In some aspects, the first Fc polypeptide and the second Fc polypeptide are linked or associated by a peptide bond. In another aspect, the first Fc polypeptide and the second Fc polypeptide are linked or associated by a disulfide bond. In some aspects, the first Fc polypeptide and the second Fc polypeptide are linked or associated by one or more amino acids. In some aspects, the first Fc polypeptide and the second Fc polypeptide are linked or associated by a peptide linker. Any linker known in the art and/or disclosed herein can be used to link the first Fc polypeptide and the second Fc polypeptide. In some aspects, the first polypeptide and the second polypeptide are linked by a disulfide bond between the first Fc polypeptide and the second Fc polypeptide. In some aspects, the first polypeptide and the second polypeptide are linked by a peptide linker between the C terminus of the first Fc polypeptide and the N terminus of the second Fc polypeptide. In some aspects, the first polypeptide and the second polypeptide are linked by a peptide linker between the N terminus of the first Fc polypeptide and the C terminus of the second Fc polypeptide. In some aspects, the first polypeptide and the second polypeptide are linked by a peptide linker between the C terminus of the first Fc polypeptide and the N terminus of the second IL-10 polypeptide. In some aspects, the first polypeptide and the second polypeptide are linked by a peptide linker between the N terminus of the first Fc polypeptide and the C terminus of the second IL-10 polypeptide. In some aspects, the first polypeptide and the second polypeptide are linked by a single chain Fc region. In some aspects, the linker between the first polypeptide and the second polypeptide is a cleavable linker.

In some aspects, the second polypeptide comprises a second IL-10 polypeptide fused to a second Fc polypeptide. In some aspects, the IL-10 polypeptide of the first polypeptide is the same as the second IL-10 polypeptide. In some aspects, the IL-10 polypeptide of the first polypeptide is different from the second IL-10 polypeptide. In some aspects, the Fc polypeptide of the first polypeptide is the same as the second Fc polypeptide. In some aspects, the Fc polypeptide of the first polypeptide is different from the second Fc polypeptide. In some aspects, the IL-10 polypeptide is the same as the second IL-10 polypeptide, and the Fc polypeptide is the same as the second Fc polypeptide. In some aspects, the IL-10 polypeptide is the same as the second IL-10 polypeptide, and the Fc polypeptide is different from the second Fc polypeptide. In some aspects, the IL-10 polypeptide is different from the second IL-10 polypeptide, and the Fc polypeptide is the same as the second Fc polypeptide. In some aspects, the IL-10 polypeptide is different from the second IL-10 polypeptide, and the Fc polypeptide is different from the second Fc polypeptide. In some aspects, the dimer is a homodimer. In some aspects, the dimer is a heterodimer.

In some aspects, the IL-10 dimer comprises a first polypeptide and a second polypeptide, wherein the first polypeptide comprises any IL-10 fusion protein described herein, and wherein the second polypeptide comprises a second IL-10 polypeptide. In some aspects, the second polypeptide does not comprise a second Fc polypeptide.

### II.F. Polynucleotides

In certain aspects, provided herein are polynucleotides, e.g., DNA or RNA, comprising a nucleotide sequence encoding a fusion protein described herein that has IL-10 activity, as well as vectors comprising such polynucleotide sequences, e.g., expression vectors for their efficient expression in host cells, e.g., mammalian cells. In some aspects, provided herein are polynucleotide sequences that encode a polypeptide sequence selected from SEQ ID NOs: 14-36.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific aspect, a nucleic acid molecule(s) encoding a fusion protein described herein is isolated or purified.

The polynucleotides can be obtained, and the nucleotide sequence of the polynucleotides determined, by any method known in the art. Nucleotide sequences encoding fusion proteins described herein, e.g., the fusion proteins described in Table 4, and modified versions of these fusion proteins can be determined using methods well known in the art, i.e., nucleotide codons known to encode particular amino acids are assembled in such a way to generate a nucleic acid that encodes the fusion protein. Such a polynucleotide encoding the fusion protein can be assembled from chemically synthesized oligonucleotides (e.g., as described in Kutmeier G et al., (1994), BioTechniques 17: 242-6), which, briefly, involves the synthesis of overlapping oligonucleotides containing portions of the sequence encoding the fusion protein, annealing and ligating of those oligonucleotides, and then amplification of the ligated oligonucleotides by PCR.

If a clone containing a nucleic acid encoding a particular fusion protein is not available, but the sequence of the fusion protein molecule is known, a nucleic acid encoding the fusion protein can be chemically synthesized or obtained from a suitable source (e.g., a cDNA library or a cDNA library generated from, or nucleic acid, in some aspects poly A+RNA, isolated from, any tissue or cells expressing the proteins of interest, such as mammalian cells expressing a part of a fusion protein described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes at least a portion of the fusion proteins. Amplified nucleic acids generated by PCR can then be cloned into replicable cloning vectors using any method well known in the art.

DNA encoding fusion proteins described herein can be readily isolated and sequenced using conventional procedures (e.g., by using oligonucleotide probes that are capable of binding specifically to genes encoding the fusion proteins disclosed herein). Human cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS SYSTEM™ (Lonza)), or myeloma cells that do not otherwise produce fusion protein, to obtain the synthesis of fusion proteins in the recombinant host cells.

It is further recognized that a polynucleotide encoding a fusion protein described herein can comprise additional elements that aid in the translation of the fusion protein. Such sequences include, for example, Kozak sequences attached to the 5′ end of the polynucleotide encoding the fusion protein.

### II.G. Cells and Vectors

In certain aspects, provided herein are cells (e.g., host cells) expressing (e.g., recombinantly) fusion proteins described herein and expression vectors comprising nucleotides that encode fusion proteins described herein. Provided herein are vectors (e.g., expression vectors) comprising polynucleotides comprising nucleotide sequences encoding a fusion protein for recombinant expression in host cells.

In some aspects, the host cell comprises the nucleic acids described herein.

In some aspects, the host cell is a eukaryotic cell. In some aspects, the host cell is selected from the group consisting of a mammalian cell, an insect cell, a yeast cell, a transgenic mammalian cell, and a plant cell. In some aspects, the host cell is a prokaryotic cell. In some aspects, the prokaryotic cell is a bacterial cell.

In some aspects, the host cell is a mammalian cell. Such mammalian host cells include but are not limited to CHO, VERO, BHK, HeLa, MDCK, HEK293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP2/0, R1.1, B-W, L-M, BSC1, BSC40, YB/20, BMT10, and HsS78Bst cells. In certain aspects, the fusion proteins are expressed in HEK293 cells. In certain aspects, the fusion proteins are expressed in CHO cells.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

A gene expression control sequence as used herein is any regulatory nucleotide sequence, such as a promoter sequence or promoter-enhancer combination, which facilitates the efficient transcription and translation of the coding nucleic acid to which it is operably linked. The gene expression control sequence may, for example, be a mammalian or viral promoter, such as a constitutive or inducible promoter.

For the purposes of this disclosure, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. In some aspects, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

In other aspects the polypeptides of the instant disclosure are expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art, as discussed above. The transformed cells are grown under conditions appropriate for the production of the fusion protein, and assayed for fusion protein synthesis. Exemplary assay techniques include enzyme-linked immunosorbent assay (ELISA), radioimmunoassay (RIA), or fluorescence-activated cell sorter analysis (FACS), immunohistochemistry, and the like.

### II.H. Pharmaceutical Compositions

The various fusion proteins disclosed herein comprising an IL-10 polypeptide and an Fc polypeptide can be incorporated into pharmaceutical compositions suitable for administration. Such compositions typically comprise the fusion protein and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art.

In some aspects, disclosed is a pharmaceutical composition comprising (a) a fusion protein as described herein and (b) a pharmaceutically acceptable excipient.

In some aspects, disclosed is a pharmaceutical composition comprising (a) a nucleic acid or a set of nucleic acids as described herein and (b) a pharmaceutically acceptable excipient.

In some aspects, disclosed is a pharmaceutical composition comprising (a) a vector or a set of vectors as described herein and (b) a pharmaceutically acceptable excipient.

In some aspects, disclosed is a pharmaceutical composition comprising (a) a host cell as described herein and (b) a pharmaceutically acceptable excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal. In addition, it may be desirable to administer a therapeutically effective amount of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In another aspect, the therapeutically effective amount of the pharmaceutical composition is delivered in a vesicle, such as liposomes (see, e.g., Langer, Science 249:1527-33, 1990 and Treat et al., in Liposomes in the Therapy of Infectious Disease and Cancer, Lopez Berestein and Fidler (eds.), Liss, N.Y., pp. 353-65, 1989).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Solutions or suspensions used for parenteral, intradermal, or subcutaneous application can include any of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELS (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the fusion protein in the required amount in an appropriate solvent with one or a combination of ingredients enumerated above, as required, followed by filtered sterilization. Generally, dispersions are prepared by incorporating the fusion protein into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a lyophilized powder of the active ingredient, e.g., the fusion protein, plus any additional desired ingredient from a previously sterile-filtered solution thereof. In some aspects, the fusion protein may be lyophilized for storage and reconstituted before administration to a subject in need thereof.

For administration by inhalation, the compounds are delivered in the form of an aerosol spray from a pressurized container or dispenser that contains a suitable propellant, e.g., a gas such as carbon dioxide, or a nebulizer. Systemic administration can also be by transmucosal or transdermal means.

In one aspect, the fusion protein can be prepared with carriers that will protect it against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers.

It is especially advantageous to formulate parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the disclosure are dictated by and directly dependent on the unique characteristics of the fusion protein and the particular therapeutic effect to be achieved. The pharmaceutical compositions can be included in a container, pack, or dispenser together with instructions for administration.

### III. Methods of Treatment

Certain aspects of the present disclosure are directed to methods of treating a disease or condition in a subject in need thereof, comprising administering a fusion protein disclosed herein to the subject. Some aspects of the disclosure are directed to methods of treating a cancer in a subject in need thereof, comprising administering to the subject an effective amount of a IL-10 fusion protein disclosed herein. Some aspects of the disclosure are directed to methods of killing a cancer cell in a subject in need thereof, comprising administering to the subject an effective amount of an IL-10 fusion protein disclosed herein.

Certain aspects of the present disclosure are directed a method of treating cancer in a subject in need thereof, comprising administering to the subject an effective amount of an IL-10 fusion protein at a dosing interval at least about 7 days, wherein the IL-10 fusion protein comprises an IL-10 polypeptide and a second polypeptide, which comprises an albumin polypeptide or an Fc polypeptide. Certain aspects of the present disclosure are directed a method of killinbg a cancer call in a subject in need thereof, comprising administering to the subject an effective amount of an IL-10 fusion protein at a dosing interval at least about 7 days, wherein the IL-10 fusion protein comprises an IL-10 polypeptide and a second polypeptide, which comprises an albumin polypeptide or an Fc polypeptide. In some aspects, the second polypeptide is an albumin polypeptide. In some aspects, the second polypeptide is an Fc polypeptide. In some aspects, the IL-10 fusion protein further comprises a linker.

In some aspects, the fusion proteins disclosed herein comprising an IL-10 polypeptide and an Fc polypeptide have a longer half-life than an IL-10 not fused with an Fc polypeptide (e.g., wild-type human IL-10), when administered to a human subject. In some aspects, the half-life of the fusion protein is at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, at least about 30 fold, or at least about 40 fold higher than an IL-10 not fused to an Fc polypeptide (e.g., wild-type human IL-10). In some aspects, the IL-10 fusion protein is an Fc-IL-10 fusion protein. In some aspects, the IL-10 fusion protein is an IL-10-Fc fusion protein.

Because the half-life of the IL-10 fusion proteins disclosed herein is longer than that of the other IL-10 proteins, the clinical benefit experienced by the subject has a longer duration than other known IL-10 proteins. As a result, the fusion proteins disclosed herein can be administered less frequently, e.g., at a higher dosing interval, than other known IL-10 proteins. In some aspects, the IL-10 fusion protein is administered at a dosing interval of at least about 7 days to at least about 28 days, at least about 7 days to at least about 21 days, at least about 7 days to at least about 14 days, at least about 10 days to at least about 28 days, at least about 10 days to at least about 21 days, at least about 10 days to at least about 14 days, at least about 14 days to at least about 28 days, at least about 14 days to at least about 21 days, or at least about 21 days to at least about 28 days. In some aspects, the IL-10 fusion protein is administered at a dosing interval of at least about 7 days to at least about 14 days. In some aspects, the IL-10 fusion protein is administered at a dosing interval of at least about 14 days to at least about 28 days. In some aspects, the IL-10 fusion protein is administered at a dosing interval of at least about 14 days to at least about 21 days. In some aspects, the IL-10 fusion protein is administered at a dosing interval of at least about 21 days to at least about 28 days.

In some aspects, the IL-10 fusion protein is administered at a dosing interval of at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days. In some aspects, the IL-10 fusion protein is administered at a dosing interval of at least about 7 days. In some aspects, the IL-10 fusion protein is administered at a dosing interval of at least about 8 days. In some aspects, the IL-10 fusion protein is administered at a dosing interval of at least about 9 days. In some aspects, the IL-10 fusion protein is administered at a dosing interval of at least about 10 days. In some aspects, the IL-10 fusion protein is administered at a dosing interval of at least about 11 days. In some aspects, the IL-10 fusion protein is administered at a dosing interval of at least about 12 days. In some aspects, the IL-10 fusion protein is administered at a dosing interval of at least about 13 days. In some aspects, the IL-10 fusion protein is administered at a dosing interval of at least about 14 days. In some aspects, the IL-10 fusion protein is administered at a dosing interval of at least about 15 days. In some aspects, the IL-10 fusion protein is administered at a dosing interval of at least about 16 days. In some aspects, the IL-10 fusion protein is administered at a dosing interval of at least about 17 days. In some aspects, the IL-10 fusion protein is administered at a dosing interval of at least about 18 days. In some aspects, the IL-10 fusion protein is administered at a dosing interval of at least about 19 days. In some aspects, the IL-10 fusion protein is administered at a dosing interval of at least about 20 days. In some aspects, the IL-10 fusion protein is administered at a dosing interval of at least about 21 days. In some aspects, the IL-10 fusion protein is administered at a dosing interval of at least about 22 days. In some aspects, the IL-10 fusion protein is administered at a dosing interval of at least about 23 days. In some aspects, the IL-10 fusion protein is administered at a dosing interval of at least about 24 days. In some aspects, the IL-10 fusion protein is administered at a dosing interval of at least about 25 days. In some aspects, the IL-10 fusion protein is administered at a dosing interval of at least about 26 days. In some aspects, the IL-10 fusion protein is administered at a dosing interval of at least about 27 days. In some aspects, the IL-10 fusion protein is administered at a dosing interval of at least about 28 days.

In some aspects, the IL-10 fusion protein is administered no more than once a week, no more than once every two weeks, no more than once every three weeks, or no more than once every four weeks. In some aspects, the IL-10 fusion protein is administered no more than once a week. In some aspects, the IL-10 fusion protein is administered no more than once every two weeks. In some aspects, the IL-10 fusion protein is administered no more than once every three weeks. In some aspects, the IL-10 fusion protein is administered no more than once every four weeks. In some aspects, the IL-10 fusion protein is administered no more than once every month.

In some aspects, the IL-10 fusion protein is administered about once a week, once about every two weeks, once about every three weeks, once about every four weeks, once about every five weeks, once about every six weeks, once about every seven weeks, or once about every eight weeks. In some aspects, the IL-10 fusion protein is administered about once a week. In some aspects, the IL-10 fusion protein is administered once about every two weeks. In some aspects, the IL-10 fusion protein is administered once about every three weeks. In some aspects, the IL-10 fusion protein is administered once about every four weeks. In some aspects, the IL-10 fusion protein is administered once about every month. In some aspects, the IL-10 fusion protein is administered once about every two months.

The IL-10 fusion protein disclosure herein can be administered as a single dose or as multiple doses. In some aspects, the IL-10 fusion protein is administered as a single dose. In some aspects, an effective amount of the IL-10 fusion protein consists essentially of or consists of a single dose.

In some aspects, the the IL-10 fusion protein is administered at a dose ranging from 0.001 mg/kg to 10.0 mg/kg body weight once every week or every 2, 3, 4, 5, 6, 7, or 8 weeks, e.g., 0.002 mg/kg to 1.0 mg/kg body weight once every week or every 2, 3, or 4 weeks. In some aspects, the IL-10 fusion protein is administered at a dose ranging from about 0.001 mg/kg to about 0.5 mg/kg. In some aspects, the IL-10 fusion protein is administered at a dose ranging from about 0.01 mg/kg to about 0.25 mg/kg. In some aspects, the IL-10 fusion protein is administered at a dose ranging from about 0.01 mg/kg to about 0.1 mg/kg. In some aspects, the IL-10 fusion protein is administered at a dose ranging from about 0.1 mg/kg to about 0.2 mg/kg. In some aspects, the IL-10 fusion protein is administered at a dose ranging from about 0.01 mg/kg to about 0.03 mg/kg, from about 0.03 mg/kg to about 0.06 mg/kg, from about 0.06 mg/kg to about 0.1 mg/kg, from about 0.1 mg/kg to about 0.15 mg/kg, from about 0.15 mg/kg to about 0.18 mg/kg, from about 0.18 mg/kg to about 0.2 mg/kg, from about 0.2 mg/kg to about 0.25 mg/kg, from about 0.25 mg/kg to about 0.3 mg/kg, or from about 0.3 mg/kg to about 0.5 mg/kg. In other aspects, the IL-10 fusion protein is administered at a dose of about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.30 mg/kg, about 0.31 mg/kg, about 0.32 mg/kg, about 0.33 mg/kg, about 0.34 mg/kg, about 0.35 mg/kg, about 0.38 mg/kg, about 0.4 mg/kg, about 0.5 mg/kg, about 0.6 mg/kg, about 0.7 mg/kg, about 0.8 mg/kg, about 0.9 mg/kg, about 1 mg/kg, 2 mg/kg, or 3 mg/kg body weight once every week or every 2, 3, 4 or 8 weeks.

In other aspects, the IL-10 fusion protein is administered at a dose of about 0.005 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.2 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.4 mg/kg, or about 0.5 mg/kg body weight once every 2 weeks. In other aspects, the IL-10 fusion protein is administered at a dose of about 0.005 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.2 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.4 mg/kg, or about 0.5 mg/kg once every 3 weeks. In other aspects, the IL-10 fusion protein is administered at a dose of about 0.05 mg/kg, about 0.005 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.2 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.4 mg/kg, or about 0.5 mg/kg weight once every 4 weeks. In other aspects, the IL-10 fusion protein is administered at a dose of about 0.005 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.2 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.4 mg/kg, or about 0.5 mg/kg body weight once about every 6 weeks. In one aspect, the IL-10 fusion protein is administered at a dose of about 0.1 mg/kg body weight about once every week. In one aspect, the IL-10 fusion protein is administered at a dose of about 0.1 mg/kg body weight about once every 2 weeks. In one aspect, the IL-10 fusion protein is administered at a dose of about 0.1 mg/kg body weight about once every 3 weeks. In one aspect, the IL-10 fusion protein is administered at a dose of about 0.1 mg/kg body weight about once every 4 weeks.

The IL-10 fusion protein useful for the present disclosure can be administered as a flat dose. In some aspects, the IL-10 fusion protein is administered at a flat dose of from about 0.1 mg to about 1000 mg, from about 0.5 mg to about 500 mg, from about 1 mg to about 200 mg, from about 1 mg to about 100 mg, from about 1 mg to about 50 mg, from about 2 mg to about 50 mg, from about 2 mg to about 40 mg, from about 2 mg to about 30 mg, from about 2 mg to about 20 mg, from about 2 mg to about 15 mg, from about 2 mg to about 10 mg, from about 3 mg to about 30 mg, from about 3 mg to about 20 mg, from about 3 mg to about 15 mg, from about 3 mg to about 10 mg, from about 4 mg to about 30 mg, from about 4 mg to about 20 mg, from about 4 mg to about 15 mg, or from about 4 mg to about 10 mg.

In some aspects, the IL-10 fusion protein is administered at a flat dose of from about 0.5 mg to about 1 mg, from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, from about 9 mg to about 10 mg, from about 10 mg to about 11 mg, from about 11 mg to about 12 mg, from about 12 mg to about 13 mg, from about 13 mg to about 14 mg, from about 14 mg to about 15 mg, from about 15 mg to about 16 mg, from about 16 mg to about 17 mg, from about 17 mg to about 18 mg, from about 18 mg to about 20 mg, or from about 20 mg to about 25 mg.

In some aspects, the IL-10 fusion protein is administered at a flat dose of from about 0.1 mg to about 0.8 mg, from about 0.8 mg to about 2 mg, from about 2 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 60 mg, from about 60 mg to about 100 mg.

In one aspect, the IL-10 fusion protein is administered as a flat dose of at least about 0.5 mg, at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, or at least about 29 mg, at least about 30 mg, at least about 35 mg, at least abou 40 mg, at least about 45 mg, at least about 50 mg, or at least about 60 mg, at a dosing interval of about 1, 2, 3, 4, 5, 6, 7, 8, 9, or 10 weeks. In one aspect, the IL-10 fusion protein is administered as a flat dose of about 0.5 mg, 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, or about 29 mg, about 30 mg, about 35 mg, abou 40 mg, about 45 mg, about 50 mg, or about 60 mg, at a dosing interval of about 1, 2, 3, 4, 5, 6, 7, 8, 9, or 10 weeks. In some aspects, the IL-10 fusion protein is administered as a flat dose about once every 2 weeks. In some aspects, the IL-10 fusion protein is administered as a flat dose about once every 3 weeks. In some aspects, the IL-10 fusion protein is administered as a flat dose about once every 4 weeks.

In some aspects, the IL-10 fusion protein is administered as a flat dose of about 1 mg at about once every 2, 3 or 4 weeks. In other aspects, the IL-10 fusion protein is administered as a flat dose of about 5 mg at about once every 2, 3 or 4 weeks. In other aspects, the IL-10 fusion protein is administered as a flat dose of about 10 mg at about once every 2, 3 or 4 weeks. In other aspects, the IL-10 fusion protein is administered as a flat dose of about 15 mg at about once every 2, 3 or 4 weeks. In certain aspects, IL-10 fusion protein is administered as a flat dose of about 20 mg at about once every 2, 3 or 4 weeks.

### III.A. Methods of Treating a Cancer

Certain aspects of the present disclosure are directed to methods of treating disease or condition in a subject in need thereof, comprising administering an IL-10 fusion protein disclosed herein. In some aspects, the disease or condition comprises a cancer. The compositions and methods disclosed herein may be used to treat any cancer known in the art. In some aspects, the cancer comprises a tumor. In some aspects, the cancer comprises a solid tumor. In some aspects, the cancer comprises a blood-based cancer, e.g., a leukemia or a lymphoma.

In some aspects, the cancer is selected from small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC or SCCHN), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, and combinations of said cancers.

In some aspects, the cancer is selected from acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

In certain aspects, the cancer is selected from RCC, NSCLC, gastric cancer, HCC, SCCHN, and any combinations of said cancers. In some aspects, the cancer is selected from melanoma, bladder cancer, pancreatic cancer, colon cancer, SCLC, mesothelioma, hepatocellular carcinoma, prostate cancer, multiple myeloma, and combinations of said cancers. In some aspects, the cancer comprises an RCC. In some aspects, the cancer comprises an NSCLC. In some aspects, the cancer comprises a gastric cancer. In some aspects, the cancer comprises an HCC. In some aspects, the cancer comprises an SCCHN. In some aspects, the cancer comprises a melanoma. In some aspects, the cancer comprises a lymphoma. In some aspects, the cancer comprises a leukemia. In some aspects, the cancer comprises a bladder cancer. In some aspects, the cancer comprises a pancreatic cancer. In some aspects, the cancer comprises a colon cancer. In some aspects, the cancer comprises an SCLC. In some aspects, the cancer comprises a mesothelioma. In some aspects, the cancer comprises a hepatocellular carcinoma. In some aspects, the cancer comprises a prostate cancer. In some aspects, the cancer comprises a multiple myeloma.

In some aspects, the cancer is refractory. In some aspects, the cancer is relapsed. In some aspects, the cancer is metastatic. In some aspects, the cancer is advanced. In some aspects, the cancer is locally advanced.

In some aspects, the subject received a previous therapy to treat a cancer. In some aspects, the previous therapy was a standard of care therapy for the treatment of the particular cancer. In some aspects, the prior therapy comprises an immunotherapy, a chemotherapy, or a combination thereof. In some aspects, the previous therapy comprises an autologous stem cell transplantation. In some aspects, the previous therapy comprises a chimeric antigen receptor T cell (CAR-T cell) therapy. In some aspects, the previous therapy comprises a steroid, a cytotoxic agent, an immunomodulatory agent, or any combination thereof.

In some aspects, the subject received at least one, at least two, at least three, at least four, at least five, at least six, at least seven, at least eight, at least nine, or at least ten prior therapies.

### III.B. Combination Therapies

In certain aspects of the present disclosure, the method comprises administering an IL-10 fusion protein disclosed herein to a subject in need thereof in combination with a second therapeutic agent, e.g., a second anticancer therapy. In some aspects, the second therapeutic agent may be selected from an immunotherapy, a chemotherapy, a radiation therapy, a surgery, an agent that activates innate immune cells, an agent that enhances survival of NK and/or CD8+ T-cells, and any combination thereof. In some aspects, the second therapeutic agent, e.g., the second anticancer therapy, comprises an effective amount of an antibody or an antigen-binding fragment thereof that specifically binds a protein selected from Inducible T cell Co-Stimulator (ICOS), CD137 (4-1BB), CD134 (OX40), NKG2A, CD27, CD38, CD73, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-8, B7-H4, Fas ligand, CXCR4, mesothelin, CEACAM-1, CD52, HER2, SLAMF7, BCMA, MICA, MICB, CCR8, and any combination thereof.

In some aspects the immunotherapy comprises administering an immune modulator, such as a checkpoint inhibitor. Any immune modulater known in the art can be used in the methods disclosed herein. In some aspects, the checkpoint inhibitor is any reagent that modulates, i.e., blocks, inhibits, reduces, or increases, the activity of one or more checkpoint protein. In some aspects, the checkpoint protein is selected from the group selected from the group consisting of PD-1, PD-L1, CTLA-4, LAG3, TIGIT, NKG2a, OX40, ICOS, CD137, KIR, TGFβ, IL-8, IL-2, CD96, VISTA, B7-H4, Fas ligand, CXCR4, mesothelin, CD27, GITR, and any combination thereof. In some aspects, the checkpoint inhibitor or agonist modulates the activity of PD-1. In some aspects, the checkpoint inhibitor modulates the activity of PD-L1. In some aspects, the checkpoint inhibitor modulates the activity of CTLA-4. In some aspects, the checkpoint inhibitor modulates the activity of LAG3. In some aspects, the checkpoint inhibitor modulates the activity of TIGIT. In some aspects, the checkpoint inhibitor modulates the activity of TIM3. In some aspects, the checkpoint inhibitor modulates the activity of NKG2a. In some aspects, the checkpoint inhibitor modulates the activity of OX40. In some aspects, the checkpoint inhibitor modulates the activity of ICOS. In some aspects, the checkpoint inhibitor modulates the activity of CD137. In some aspects, the checkpoint inhibitor modulates the activity of KIR. In some aspects, the checkpoint inhibitor modulates the activity of TGFβ. In some aspects, the checkpoint inhibitor modulates the activity of IL-8. In some aspects, the checkpoint inhibitor modulates the activity of IL-2. In some aspects, the checkpoint inhibitor modulates the activity of CD96. In some aspects, the checkpoint inhibitor modulates the activity of VISTA. In some aspects, the checkpoint inhibitor modulates the activity of B7-H4. In some aspects, the checkpoint inhibitor modulates the activity of Fas ligand. In some aspects, the checkpoint inhibitor modulates the activity of CXCR4. In some aspects, the checkpoint inhibitor modulates the activity of mesothelin. In some aspects, the checkpoint inhibitor modulates the activity of CD27. In some aspects, the checkpoint inhibitor modulates the activity of GITR.

Any checkpoint inhibitor can be used in the methods disclosed herein. In some aspects, the checkpoint inhibitor is a small molecule. In some aspects, the checkpoint inhibitor is a protein. In some aspects, the checkpoint inhibitor is an antibody or an antigen-binding portion thereof. In some aspects, the checkpoint inhibitor is an antibody or antigen-binding portion thereof that specifically binds PD-1. In some aspects, the checkpoint inhibitor is an antibody or antigen-binding portion thereof that specifically binds CTLA-4. In some aspects, the checkpoint inhibitor is an antibody or antigen-binding portion thereof that specifically binds LAG3. In some aspects, the checkpoint inhibitor is an antibody or antigen-binding portion thereof that specifically binds TIGIT. In some aspects, the checkpoint inhibitor is an antibody or antigen-binding portion thereof that specifically binds TIM3. In some aspects, the checkpoint inhibitor is an antibody or antigen-binding portion thereof that specifically binds NKG2a. In some aspects, the checkpoint inhibitor is an antibody or antigen-binding portion thereof that specifically binds OX40. In some aspects, the checkpoint inhibitor is an antibody or antigen-binding portion thereof that specifically binds ICOS. In some aspects, the checkpoint inhibitor is an antibody or antigen-binding portion thereof that specifically binds CD137. In some aspects, the checkpoint inhibitor is an antibody or antigen-binding portion thereof that specifically binds KIR. In some aspects, the checkpoint inhibitor is an antibody or antigen-binding portion thereof that specifically binds TGFβ. In some aspects, the checkpoint inhibitor is an antibody or antigen-binding portion thereof that specifically binds IL-8. In some aspects, the checkpoint inhibitor is an antibody or antigen-binding portion thereof that specifically binds IL-2. In some aspects, the checkpoint inhibitor is an antibody or antigen-binding portion thereof that specifically binds CD96. In some aspects, the checkpoint inhibitor is an antibody or antigen-binding portion thereof that specifically binds VISTA. In some aspects, the checkpoint inhibitor is an antibody or antigen-binding portion thereof that specifically binds B7-H4. In some aspects, the checkpoint inhibitor is an antibody or antigen-binding portion thereof that specifically binds Fas ligand. In some aspects, the checkpoint inhibitor is an antibody or antigen-binding portion thereof that specifically binds CXCR4. In some aspects, the checkpoint inhibitor is an antibody or antigen-binding portion thereof that specifically binds mesothelin. In some aspects, the checkpoint inhibitor is an antibody or antigen-binding portion thereof that specifically binds CD27. In some aspects, the checkpoint inhibitor is an antibody or antigen-binding portion thereof that specifically binds GITR. In some aspects, the checkpoint inhibitor is an antibody or antigen-binding portion thereof that specifically binds MICA or MICB. In some aspects, the checkpoint inhibitor is an antibody or antigen-binding portion thereof that specifically binds CCR8. In some aspects, the checkpoint inhibitor is an antibody or antigen-binding portion thereof that specifically binds BCMA.

In some aspects, the subject is administered a combination therapy, e.g., wherein the subject is administered an IL-10 fusion protein disclosed herein and a checkpoint inhibitor. In some aspects, the subject is administered a combination therapy, e.g., wherein the subject is administered an IL-10 fusion protein disclosed herein and an anti-PD-1 antibody. In some aspects, the subject is administered a combination therapy, e.g., wherein the subject is administered an IL-10 fusion protein disclosed herein and an anti-PD-L1 antibody. In some aspects, the subject is administered a combination therapy, e.g., wherein the subject is administered an IL-10 fusion protein disclosed herein and an anti-CTLA-4 antibody. In some aspects, the subject is administered a combination therapy, e.g., wherein the subject is administered an IL-10 fusion protein disclosed herein and an anti-LAG-3 antibody. In some aspects, the subject is administered a combination therapy, e.g., wherein the subject is administered (i) an IL-10 fusion protein disclosed herein, (ii) an anti-PD-1 antibody, and (iii) an anti-CTLA-4 antibody. In some aspects, the subject is administered a combination therapy, e.g., wherein the subject is administered (i) an IL-10 fusion protein disclosed herein, (ii) an anti-PD-L1 antibody, and (iii) an anti-CTLA-4 antibody. The anticancer therapies in a combination therapy may be administered concurrently or sequentially, in any order. In some aspects, the subject is further administered an additional anticancer therapy, e.g., a chemotherapy, a radiation therapy, CAR-T therapy, gene therapy, and/or a surgery.

### III.B.1. Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449).

In another aspect, the anti-PD-1 antibody is pembrolizumab. Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Anti-PD-1 antibodies usable in the compositions and methods of the disclosed disclosure also include antigen-binding portions of the above antibodies.

In some aspects, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one aspect, the anti-PD-1 antibody is administered at a dose of about 5 mg/kg body weight about once every 3 weeks. In another aspect, the anti-PD-1 antibody, e.g., nivolumab, is administered at a dose of about 1 mg/kg or about 3 mg/kg body weight about once every 2 weeks. In other aspects, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some aspects, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one aspect, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another aspect, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some aspects, the anti-PD-1 antibody is administered as a flat dose of about 200 mg at about once every 3 weeks. In other aspects, the anti-PD-1 antibody is administered as a flat dose of about 200 mg at about once every 2 weeks. In other aspects, the anti-PD-1 antibody is administered as a flat dose of about 240 mg at about once every 2 weeks. In certain aspects, the anti-PD-1 antibody is administered as a flat dose of about 480 mg at about once every 3 weeks. In certain aspects, the anti-PD-1 antibody is administered as a flat dose of about 480 mg at about once every 4 weeks.

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 2 weeks. In some aspects, nivolumab is administered at a flat dose of about 360 mg once about every 3 weeks. In some aspects, nivolumab is administered at a flat dose of about 480 mg once about every 4 weeks.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks.

### III.B.2. Anti-PD-L1 Antibodies Useful for the Disclosure

In certain aspects, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl): TPS3113 (2018)), and CK-301 (Checkpoint Therapeutics; see Gorelik et al., AACR: Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223). In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Anti-PD-L1 antibodies usable in the compositions and methods of the disclosed disclosure also include antigen-binding portions of the above antibodies.

In some aspects, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody is administered at a dose of about 15 mg/kg body weight at about once every 3 weeks. In other aspects, the anti-PD-L1 antibody is administered at a dose of about 10 mg/kg body weight at about once every 2 weeks.

In other aspects, the anti-PD-L1 antibody useful for the present disclosure is a flat dose. In some aspects, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody is administered as a flat dose of about 1200 mg at about once every 3 weeks. In other aspects, the anti-PD-L1 antibody is administered as a flat dose of about 800 mg at about once every 2 weeks. In other aspects, the anti-PD-L1 antibody is administered as a flat dose of about 840 mg at about once every 2 weeks.

In some aspects, atezolizumab is administered as a flat dose of about 1200 mg once about every 3 weeks. In some aspects, atezolizumab is administered as a flat dose of about 840 mg once about every 2 weeks. In some aspects, atezolizumab is administered as a flat dose of about 1200 mg once about every 3 weeks. In some aspects, atezolizumab is administered as a flat dose of about 1680 mg once about every 4 weeks.

In some aspects, avelumab is administered as a flat dose of about 800 mg once about every 2 weeks.

In some aspects, durvalumab is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

### III.B.3. Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In some aspects, the anti-CTLA-4 antibody is ipilimumab.

In some aspects, the CTLA-4 antibody is ipilimumab for use in the compositions and methods disclosed herein.

In some aspects, the CTLA-4 antibody is tremelimumab.

In some aspects, the CTLA-4 antibody is MK-1308.

In some aspects, the CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Anti-CTLA-4 antibodies usable in the compositions and methods of the disclosed disclosure also include antigen-binding portions of the above antibodies.

In some aspects, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some aspects, the anti-CTLA-4 antibody or antigen-binding portion thereof is administered at a dose of 1 mg/kg or 3 mg/kg body weight once every 3, 4, 5, or 6 weeks. In one aspect, the anti-CTLA-4 antibody or antigen-binding portion thereof is administered at a dose of 3 mg/kg body weight once every 2, 3, 4, or 6 weeks. In another aspect, the anti-CTLA-4 antibody or antigen-binding portion thereof is administered at a dose of 1 mg/kg body weight once every 2, 3, 4, or 6 weeks. In another aspect, the anti-CTLA-4 antibody or antigen-binding portion thereof is administered at a dose of 10 mg/kg body weight once every 2, 3, 4, or 6 weeks.

In some aspects, the anti-CTLA-4 antibody or antigen-binding portion thereof is administered as a flat dose. In some aspects, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 50 mg to about 1000 mg, from about 50 mg to about 900 mg, from about 50 mg to about 800 mg, from about 50 mg to about 700 mg, from about 50 mg to about 100 mg, from about 50 mg to about 500 mg, from about 100 mg to about 480 mg, or from about 240 mg to about 480 mg. In one aspect, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another aspect, the anti-CTLA-4 antibody or antigen-binding portion thereof is administered as a flat dose about once every 1, 2, 3, 4, 5, 6, 7, or 8 weeks.

In some aspects, ipilimumab is administered at a dose of about 1 mg/kg once about every 3 weeks. In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses.

### III.B.4. Anti-LAG-3 Antibodies

As used herein a LAG-3 antagonist includes, but is not limited to, LAG-3 binding agents, e.g., a LAG-3 antibody, and soluble LAG-3 polypeptides, e.g., a fusion protein comprising the extracellular portion of LAG-3.

In some aspects, the LAG-3 inhibitor is a soluble LAG-3 polypeptide, for example, a LAG-3-Fc fusion polypeptide capable of binding to MHC Class II.

In some aspects, the LAG-3 antagonist comprises IMP321 (eftilagimod alpha).

In some aspects, the LAG-3 antagonist is an anti-LAG-3 antibody or an antigen binding fragment thereof that specifically binds to LAG-3 (“anti-LAG-3 antibody”).

Anti-LAG-3 antibodies (or VH/VL domains derived therefrom) suitable for use herein can be generated using methods well known in the art. Alternatively, art recognized anti-LAG-3 antibodies can be used.

In some aspects, the anti-LAG-3 antibody is a chimeric, humanized, or human monoclonal antibody, or a portion thereof. In other aspects, the anti-LAG-3 antibody is a bispecific antibody or a multispecific antibody.

In some aspects, the anti-LAG-3 antibody is relatlimab, e.g., BMS-986016 as described in PCT/US13/48999, the teachings of which are hereby incorporated by reference.

In other aspects, the antibody has the heavy and light chain CDRs or variable regions of relatlimab. Accordingly, in one aspect, the antibody comprises CDR1, CDR2, and CDR3 domains of the VH region of relatlimab, and CDR1, CDR2 and CDR3 domains of the VL region of relatlimab. In another aspect, the antibody comprises VH and/or VL regions of relatlimab. In some aspects, the anti-LAG-3 antibody cross-competes with relatlimab for binding to human LAG-3. In some aspects, the anti-LAG-3 antibody binds to the same epitope as relatlimab. In some aspects, the anti-LAG-3 antibody is a biosimilar of relatlimab. In some aspects, the anti-LAG-3 antibody is LAG-525, MK-4280, REGN3767, TSR-033, TSR-075, Sym022, FS-118, or any combination thereof. Any art recognized anti-LAG-3 antibodies can be used in the therapeutic methods of the disclosure. For example, the anti-human LAG-3 antibody described in US2011/0150892 A1, which is herein incorporated by reference, and referred to as monoclonal antibody 25F7 (also known as “25F7” and “LAG-3.1) can be used. Other art recognized anti-LAG-3 antibodies that can be used include IMP731 (H5L7BW) described in US 2011/007023, MK-4280 (28G-10) described in WO2016028672, REGN3767 described in Journal for ImmunoTherapy of Cancer, (2016) Vol. 4, Supp. Supplement 1 Abstract Number: P195, BAP050 described in WO2017/019894, IMP-701 (LAG-525), aLAG3(0414), aLAG3(0416), Sym022, TSR-033, TSR-075, XmAb22841, MGD013, BI754111, FS118, P 13B02-30, AVA-017 and GSK2831781. These and other anti-LAG-3 antibodies useful in the claimed invention can be found in, for example: U.S. Pat. No. 10,188,730, WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO18/185046, WO18/185043, WO2018/217940, WO19/011306, WO2018/208868, and WO2014/140180. The contents of each of these references are incorporated by reference herein in their entirety. Antibodies that compete with any of the art-recognized antibodies for binding to LAG-3 also can be used. In some aspects, the anti-LAG-3 antibody cross-competes with, binds to the same epitope as, or is a biosimilar of an anti-LAG-3 antibody that is described herein or that is known in the art.

In some aspects, the anti-LAG-3 antibody or antigen-binding portion thereof is administered at a dose ranging from about 0.1 mg/kg to about 10.0 mg/kg body weight once about every 1, 2, 3, 4, 5, 6, 7, or 8 weeks. In some aspects, the anti-LAG-3 antibody or antigen-binding portion thereof is administered at a dose of at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, or at least about 10 mg/kg body weight about once every 1, 2, 3, 4, 5, 6, 7, or 8 weeks. In some aspects, the anti-LAG-3 antibody or antigen-binding portion thereof is administered at a flat dose. In some aspects, the anti-LAG-3 antibody is administered at a flat dose of from about 20 mg to about 2000 mg. In one aspect, the anti-LAG-3 antibody or antigen-binding portion thereof is administered as a flat dose of at least about 80 mg or at least about 160 mg. In another aspect, the anti-LAG-3 antibody or antigen-binding portion thereof is administered at a flat dose once about every 1, 2, 3, 4, 5, 6, 7, or 8 weeks. In some aspects, the anti-LAG-3 antibody or antigen-binding portion thereof is administered at a flat dose. In some aspects, the anti-LAG-3 antibody or antigen-binding portion thereof is administered at a flat dose of about 80 mg. In some aspects, the anti-LAG-3 antibody or antigen-binding portion thereof is administered at a flat dose of about 160 mg.

### III.B.5. Additional Anticancer Therapies

In certain aspects of the present disclosure, the method comprises administering an IL-10 fusion protein disclosed herein and an additional anticancer therapy. The additional anticancer therapy can comprise any therapy known in the art for the treatment of a tumor in a subject and/or any standard-of-care therapy, as disclosed herein. In some aspects, the additional anticancer therapy comprises a surgery, a radiation therapy, a chemotherapy, an immunotherapy, or any combination thereof. In some aspects, the additional anticancer therapy comprises a chemotherapy, including any chemotherapy disclosed herein. In some aspect, the additional anticancer therapy comprises an additional immunotherapy. In some aspects, the additional anticancer therapy comprises administration of an antibody or antigen-binding portion thereof that specifically binds TIGIT, TIM3, NKG2a, OX40, ICOS, MICA, MICB, CD38, CD73, CD96, CD137, KIR, TGFβ, IL-8, B7-H4, Fas ligand, CXCR4, mesothelin, CD27, GITR, SLAMF7, BCMA, CCR8, or any combination thereof.

In some aspects, the second the second anticancer therapy comprises a chemotherapy. In some aspects, the chemotherapy is selected from a proteasome inhibitor, an IMiD, a Bet inhibitor, an IDO antagonist, a platinum-based chemotherapy, and any combination thereof. In certain aspects, the second anticancer therapy comprises a platinum-based chemotherapy.

In some aspects, the second anticancer therapy comprises an agent elected from doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, Folinic acid (leucovorin), Fluorouracil (5-FU), Oxaliplatin (Eloxatin), FOLFOX, Paclitaxel, nanoparticle albumin-bound (nab) paclitaxel (ABRAXANE®), Docetaxel, vincristine (ONCOVIN®), fludarabine (FLUDARA®), thalidomide (THALOMID®), alemtuzumab (CAMPATH®, ofatumumab (ARZERRA®), everolimus (AFINITOR®, ZORTRESS®), carfilzomib (KYPROLISTM), monolimod, marizomib, pomalidomide, Linrodostat, Bacillus Calmette-Guerin, Cabozantinib, Bempegaldesleukin, elotuzumab, daratumumab, and any combination thereof. In some aspects, the second anticancer therapy comprises doxorubicin (ADRIAMYCIN®). In some aspects, the second anticancer therapy comprises cisplatin. In some aspects, the second anticancer therapy comprises carboplatin. In some aspects, the second anticancer therapy comprises bleomycin sulfate. In some aspects, the second anticancer therapy comprises carmustine. In some aspects, the second anticancer therapy comprises chlorambucil (LEUKERAN®). In some aspects, the second anticancer therapy comprises cyclophosphamide (CYTOXAN®; NEOSAR®). In some aspects, the second anticancer therapy comprises lenalidomide (REVLIMID®). In some aspects, the second anticancer therapy comprises bortezomib (VELCADE®). In some aspects, the second anticancer therapy comprises dexamethasone. In some aspects, the second anticancer therapy comprises mitoxantrone. In some aspects, the second anticancer therapy comprises etoposide. In some aspects, the second anticancer therapy comprises cytarabine. In some aspects, the second anticancer therapy comprises bendamustine (TREANDA®). In some aspects, the second anticancer therapy comprises. In some aspects, the second anticancer therapy comprises rituximab (RITUXAN®). In some aspects, the second anticancer therapy comprises ifosfamide. In some aspects, the second anticancer therapy comprises Folinic acid (leucovorin). In some aspects, the second anticancer therapy comprises Fluorouracil (5-FU). In some aspects, the second anticancer therapy comprises Oxaliplatin (Eloxatin). In some aspects, the second anticancer therapy comprises FOLFOX. In some aspects, the second anticancer therapy comprises Paclitaxel. In some aspects, the second anticancer therapy comprises Docetaxel. In some aspects, the second anticancer therapy comprises vincristine (ONCOVIN®). In some aspects, the second anticancer therapy comprises fludarabine (FLUDARA®). In some aspects, the second anticancer therapy comprises thalidomide (THALOMID®). In some aspects, the second anticancer therapy comprises alemtuzumab (CAMPATH®, ofatumumab (ARZERRA®). In some aspects, the second anticancer therapy comprises everolimus (AFINITOR®, ZORTRESS®). In some aspects, the second anticancer therapy comprises carfilzomib (KYPROLISTM).

In some aspects, the second anticancer therapy comprises an agent that enhances survival of NK and/or CD8+ T-cells selected from pegylated IL-2, IL-18, and IL-15.

The following examples are offered by way of illustration and not by way of limitation.

### EXAMPLES

### Example 1. IL-10 and Fc Fusion

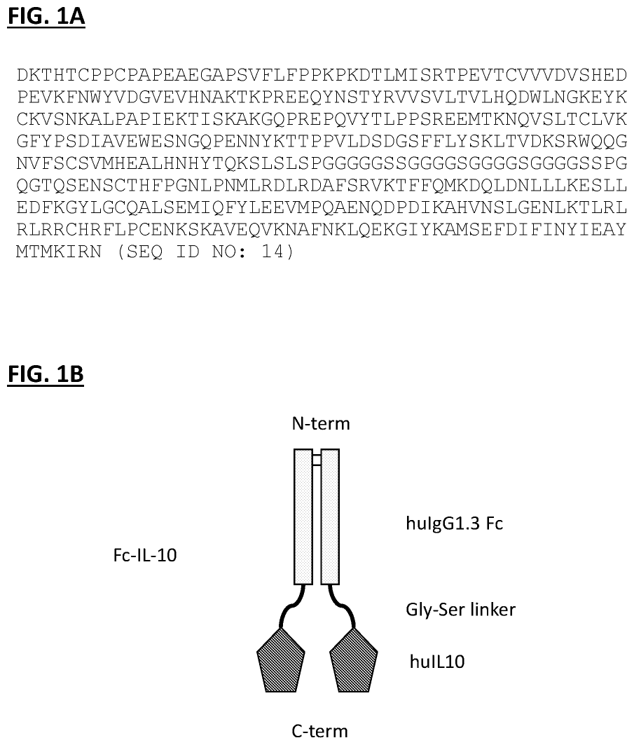

A Fc and IL-10 fusion protein was constructed by fusing a human IgG1.3f Fc domain (SEQ ID NO: 4) with a Gly-Ser rich polypeptide linker (GGGGSSGGGGSGGGGSGGGGS, SEQ ID NO: 41), and wildtype human IL-10 (SEQ ID NO: 1) to its C-terminus (Fc-IL-10, SEQ ID NO: 14) (FIGS. 1A and 1B). Another fusion protein was also made by fusing human IgG1.3f Fc domain with the wildtype human IL-10 with the Gly-Ser rich polypeptide linker to its N-terminus (IL-10-Fc, SEQ ID NO: 33). The IgG1.3f Fc domain was chosen to provide reduced antibody-dependent cellular cytotoxicity (ADCC) function while the linker serves to separate the IL-10 from the Fc domain to increase activity and enable IL-10 to form a functional dimer while attached to the Fc. The two native disulfide bonds that covalently tether the Fc dimer were retained to help prevent the disassociation of the IL-10 homodimer domain at low concentrations and low pH. The wildtype human IL-10 sequence was used without further modification to minimize immunogenic risk and to maximize the native function(s) of the protein.

The fusion proteins described herein can be expressed and prepared using routine procedures known in the art.

### Example 2. Induction of IFNγ Secretion on Primary CD8+ T Cells and NK Cytotoxicity

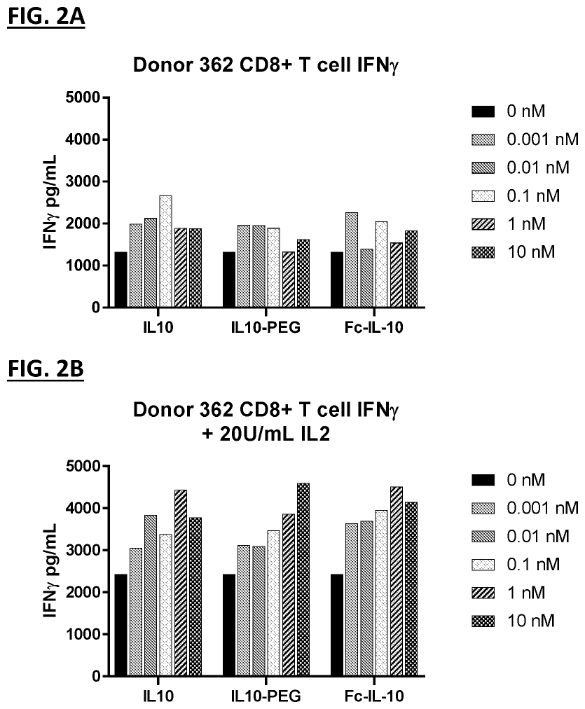

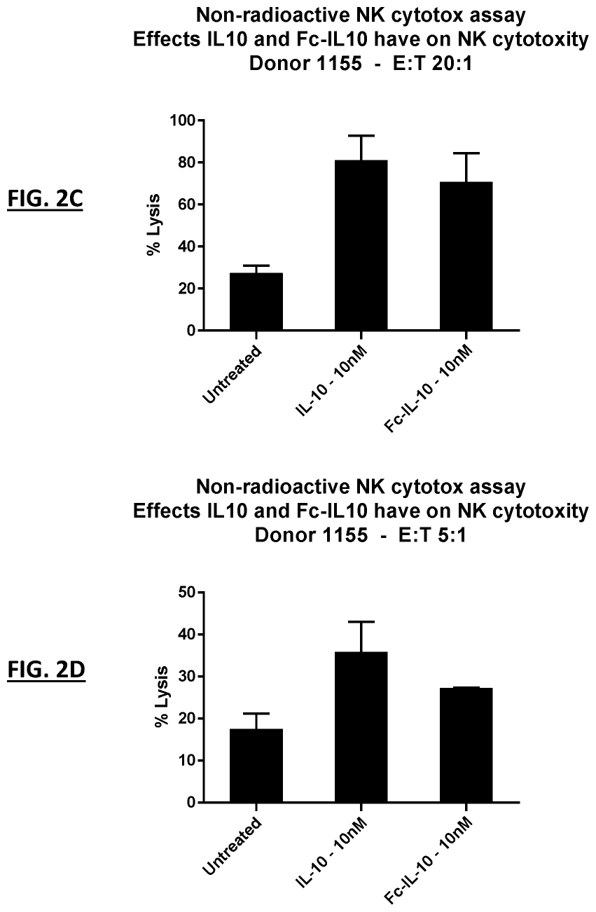

The induction of IFNγ by Fc-IL-10 on primary human CD8+ T cells was measured using Perkin Elmer IFNγ AlphaLISA detection kit. Briefly, PBMC's were isolated from human whole blood from healthy donors using density gradient centrifugation with Lympholyte H (Ceadarlane). CD8+ T cells were negatively isolated using a CD8+ T cell Isolation kit (Miltenyi). Isolated CD8+ T cells were stimulated with plate bound anti-CD3 and anti-CD28 in complete media (AIM V media (Gibco)+10% Fetal Bovine Serum (FBS) (Gibco)) for 72 hours at 37° C. in 5% CO₂. After incubation, cells were washed with Phosphate-buffered saline (Gibco), plated in complete media and rested for 3 hours at 37° C. in 5% CO₂ prior to stimulation with Fc-IL-10 or controls in complete media and with or without 20 U/mL Recombinant Human IL-2 (Peprotech) for 72 hours. Supernatant was analyzed for IFNγ using Perkin Elmer IFNγ AlphaLISA detection kit. Recombinant human IL-10, pegylated human IL-10 (PEG-IL-10), and Fc-IL-10 all induced IFNγ secretion by primary CD8+ T cells (FIGS. 2A-2B).

Induction of cell-mediated cytotoxity by Fc-IL-10 on primary human NK cells was measured using Perkin Elmer DELFIA® EuTDA Cytotoxicity Reagents (Perkin Elmer). Briefly, PBMC's were isolated using density gradient centrifugation (Lympholyte H (Ceadarlane)) from human whole blood from healthy donors. Human primary NK cells were negatively isolated using a NK cell Isolation kit (Miltenyi). Separately, K562 cells were loaded with ligand TDA (Perkin Elmer) and washed with 2 mM probenecid to prevent leaking. The NK cells were pretreated with IL-10 or Fc-IL-10 at 10 nM for 48 hours and then added to TDA-labeled K562 target cells at an E:T ratio of 20:1 or 5:1. The mixtures were incubated at 37° C. in 5% CO₂ for 2 hours. Supernatant was added to Europium solution (Perkin Elmer) and fluorescence was measured in a time-resolved fluorometer (EnVision). The measured signal correlates directly with the amount of lysed cells. Cytotoxicity was measured and plotted as percentage lysis. Human NK cells pretreated with Fc-IL-10 showed increased cytotoxicity in the K562 target killing assay (FIGS. 2C-2D), indicating that Fc-IL-10 potentiates NK-mediated cytotoxicity.

### Example 3. Phosphorylation of STAT3 in Primary Immune Cells

Human in-vitro pSTAT3 Assessment: STAT3 phosphorylation by Fc-IL-10 on T cells (CD3+) and B Cells (CD19+) in human whole blood was measured using flow cytometry. The flow cytometry panel was as follows: CD3 [UCHT1; Biolegend], CD19 [HIB19; Biolegend] and STAT3 (pY705) [4/P-STAT3; BD Biosciences]. Briefly, human whole blood from healthy donors were stimulated with Fc-IL-10 or controls (hIL-10 and 5KPEG-hIL-10 (human IL-10 conjugated to 5 KD PEG)). Stimulation was stopped by adding pre-warmed Phosflow Lyse/Fix buffer (BD biosciences) then washed in FAC Buffer (Dulbecco's phosphate-buffered saline (DPBS) (Gibco) and 0.5% Fetal Bovine Serum (FBS) (Gibco). Samples were stained with antibodies for surface markers (CD3 and CD19), permeabilized with Phosflow Perm Buffer III [BD Biosciences] and stained for internal marker with pSTAT3 antibody. After a 30-minute incubation, samples were washed, re-suspended in FACS buffer and analyzed by FACSCantoX™ flow cytometry system.

Mouse in-vitro pSTAT3 Assessment: STAT3 phosphorylation by mIgG1-D265A-Fc-(G₄S)₄-mIL-10 (“mFc-IL-10”) (SEQ ID NO: 37) in T cells (CD3+) and B Cells (CD45R+) from C57BL/6 mouse splenocytes was measured using flow cytometry. The flow cytometry panel was as follows: CD3 [17A2; BD Biosciences], CD45R [RA3-6B2; BD Biosciences] and STAT3 (pY705) [4/P-STAT3; BD Biosciences]. Briefly, splenocytes from C57BL/6 mice were mechanically disrupted, resuspended in RPMI-1640 [Gibco] and filtered through a 70 μm filter. Following lysing of the red blood cells [Sigma], splenocytes were plated in 96 well u-bottom plates and stimulated with mFc-mIL10 or controls. Stimulation was stopped by adding cold FAC Buffer (Dulbecco's phosphate-buffered saline (DPBS) [Gibco]+0.5% Fetal Bovine Serum (FBS) [Gibco] to each well. Samples were then fixed (BD Cytofix [BD Biosciences]), permeabilized with Phosflow Perm Buffer III [BD Biosciences]), blocked for non-specific binding (BD mouse FC block [BD Biosciences]), and stained with antibodies (CD3, CD45R, pSTAT3). After a 30-minute incubation, samples were washed, re-suspended in FACS buffer and analyzed by FACSCantoX™ flow cytometry system.

Treatment with Fc-IL-10 resulted in induction of phosphorylation of STAT3 as a proximal biological readout of target engagement and signaling in primary immune cells, comparable in potency to pegylated IL-10 (Table 5).

TABLE 5

| | hIL-10 | 5KPEG-hIL-10 | hFc-IL-10 |
|---|---|---|---|
| Human Cell Population | (EC₅₀, pM) | (EC₅₀, pM) | (EC₅₀, pM) |
| CD3 + T cells | 47.7 | 167.4 | 221.5 |
| CD19 + B cells | 116.3 | 347.4 | 352.2 |
| Murine Cell Population | m IL-10 | 5KPEG-mIL-10 | mFc-IL-10 |
| CD3 + T cells | 91.4 | 390.2 | 440.9 |
| CD19 + B cells | 55.3 | 120.2 | 125.4 |

### Example 4. Transcript Analysis of Mouse and Human CRC Tumor Explants

Mouse CRC tumors (MC38) (n=3) pooled, harvested from mice, or Human CRC tumors (n=1) procured, were cut into small pieces, dipped in media and placed in the wells of a 96 well plate (8 wells per treatment). The plate was then kept at 37° C. for 2 hours. 100 ul Lymphocyte growth media with freshly added 25 ng/ml (for mouse MC38 tumors) or 20 ng/ml (for human CRC tumors) IL-2 was added to each well. The plate was then incubated for 18 hours at 37° C. Then 100 ul media with or without 1 nM Fc-IL-10 (for human CRC tumors) or 0.1 nM mFc-IL-10 (for mouse MC38 tumors) was added to the appropriate wells and the plate was further incubated for 72 hrs at 37° C. Supernatant was removed from the wells; RNA lysis buffer (prepared according to the RNA Easy kit, Qiagen) was added to each well to harvest RNA from tissue and cells. RNA Easy kit (Qiagen) was used to isolate RNA from each sample and cDNA was generated using quantified RNA with Reverse transcription kit (Invitrogen). The resulting cDNA was diluted 4 times with DNase- and RNase-free water. Then 1 ul of the diluted cDNA was used per replicate for quantitative PCR to measure gene expression levels of CD8α, IFNγ, Granzyme B, and GAPDH.

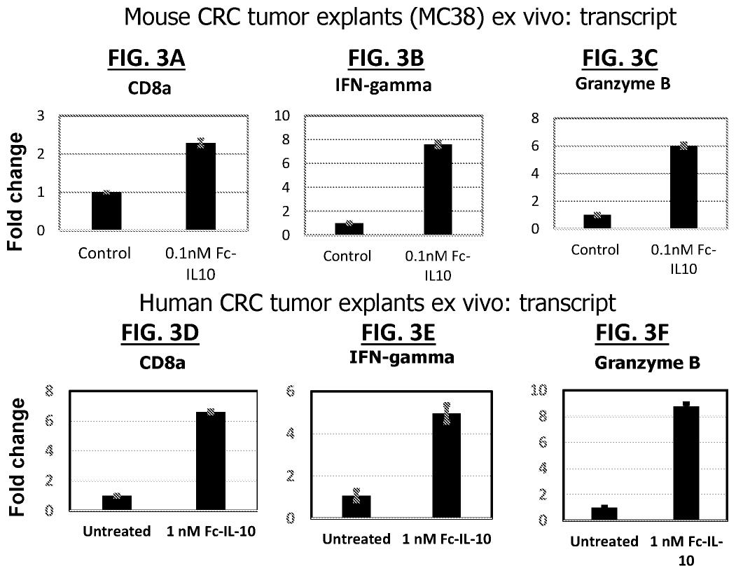

Fc-IL-10 induced the expression of CD8α, granzyme B and IFNγ in both primary mouse and human tumor explant cultures ex vivo (see FIGS. 3A-3F).

### Example 5. Treatment of Tumors in Animal Models

Female C57BL/6NCrl mice were bred and shipped from Charles River Laboratories, (for MC38-255, MC38-337). Female Balb/cAnNHsd mice were bred and shipped from Envigo, (for CT26-210, CT26-213). Mice were six to eight weeks of age upon delivery and were implanted within 1 month of arrival.

Mice received tumor implantation from a subcutaneous injection on the right flank of 1e6 viable MC-38 or CT-26 cells in a single cell suspension at a concentration of 1e7 cells/mL. Day of implantation designated as day 0 on study. Implanted animals were sorted by tumor volume and randomized into groups upon tumors reaching their approximate target initiation size, 100 mm³. Mice receiving an administration of formulated compounds were individually weighed immediately prior to each administration. Dose volume administered at 10 mL/kg of bodyweight for intraperitoneal (IP) injection, or 5 mL/kg of bodyweight for subcutaneous (SC) injection.

For pharmacokinetic (PK) analysis of mFc-IL10 exposure, 10 uL of whole blood was drawn from the tail vein via tail nick and placed into 90 uL of REXXIP Buffer A (Gyros) and then immediately placed onto wet ice, and then centrifuged 3,000 g at 4° C. for 30 minutes. 65 uL supernatant plated and stored at −20° C. until further analysis.

Tumor response was determined by measurement of tumors with a caliper twice a week. Animals remained on-study until individual tumors reached a predetermined target size of 1 cm³ in two subsequent measurements. Tumor volumes [mm³] were calculated with the formula:

Tumor volume [mm³]=(length [mm]×width [mm]²)/2

Animals with tumors reaching complete regression, measuring 0 mm³, were monitored weekly for tumor relapse. Animals with complete regressions were considered tumor free (cured) 45 days after all tumors on-study had completely regressed or reached tumor burden.

MC38 Treated with a Single Dose of mFc-IL-10:

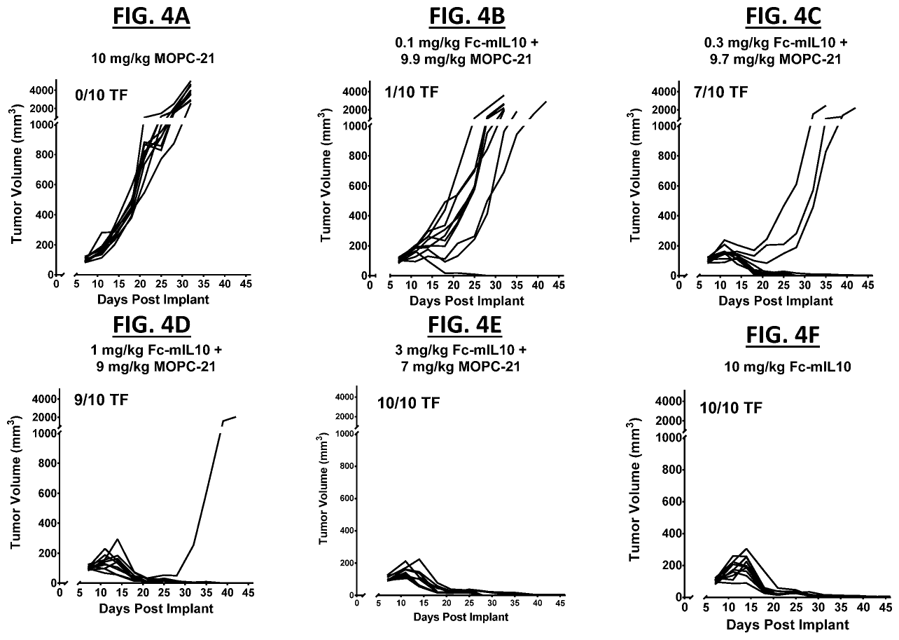

MC-38 tumor bearing C57BL/6NCrl female mice received a single IP administration (QDx1 IP) on day 7 of 0, 0.1, 0.3, 1.0, 3.0 or 10 mg/kg mFc-IL-10 in combination with a balanced isotype control MOPC-21 (murine IgG1, BioXCell) of 10, 9.9, 9.7, 9.0, 7.0, or 0 mg/kg, respectively (total of 10 mg/kg). Individual tumor volumes measured and recorded, n=10 per group. The number of tumor free mice yielded 0% (0/10), 10% (1/10), 70% (7/10), 90% (9/10), 100% (10/10) and 100% (10/10) from groups receiving 0, 0.1, 0.3, 1.0, 3.0 and 10 mg/kg of mFc-IL-10, respectively (FIGS. 4A-4F). Thus single dose monotherapy treatment with Fc-mIL10 induces dose-dependent tumor efficacy.

MC-38 Treated with a Single Dose of mFc-IL-10 or PEG-mIL-10

MC-38 tumor bearing C57BL/6NCrl female mice received a single IP administration on day 6 of: 0.1, 0.3, 1.0, 3.0 or 10 mg/kg mFc-IL-10, or equivalent IL-10 molar concentration of 10 kD PEG-mIL10 at 0.04, 0.13, 0.43, 1.28, or 4.28 mg/kg, respectively, or 10 mg/kg isotype control anti-DT mIgG1 D265A. Animals receiving Fc-mIL10 were balanced (to a total of 10 mg/kg) with a combination of 9.9, 9.7, 9.0, 7.0, or 0 mg/kg of isotype control anti-DT mIgG1 D265A. Ten mice (n=10) per group were allocated for tumor volume efficacy and tumor immune-monitoring. Blood for PK micro-sampling was taken from animals allocated for immune-monitoring.

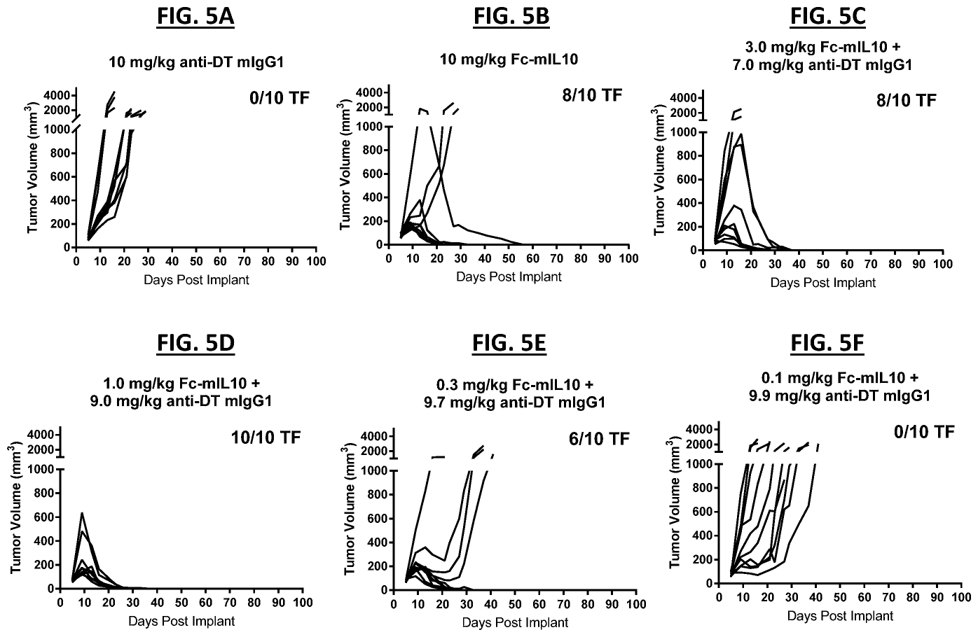

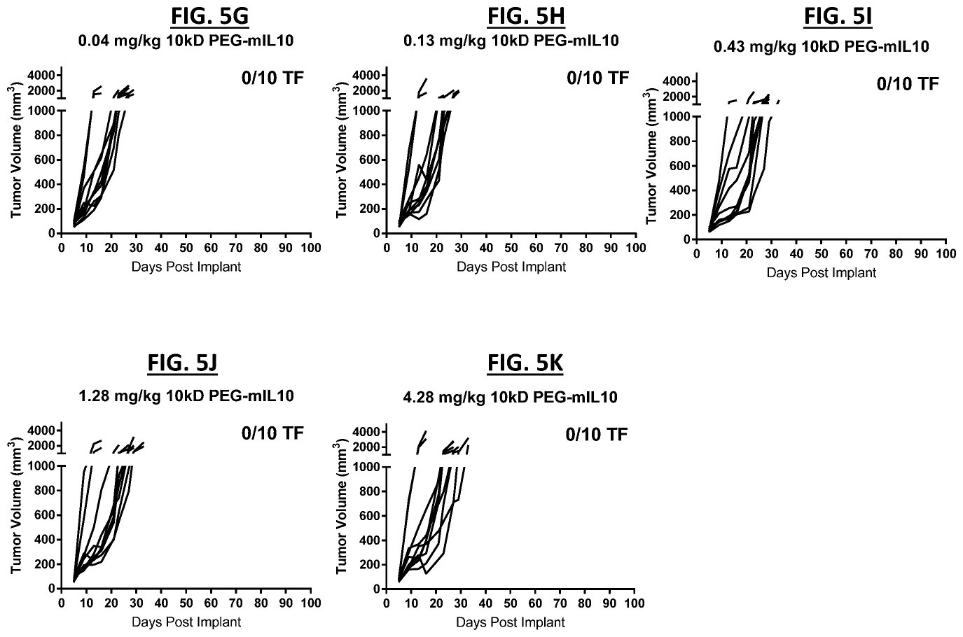

An 80-100% tumor free yield was found with groups receiving a single mFc-IL10 administration as low as 1.0 mg/kg (FIGS. 5A-5F); yet there were no tumor free animals in any of the groups receiving a single dose of 10 kD PEG-mIL10 (FIGS. 5G-5K). Thus single low dose of Fc-IL-10 treatment mediates potent efficacy while single high dose of PEG-IL-10 was minimally efficacious.

Immune Monitoring

CD8+ T cell (CD45+, CD3+, CD8+), CD4+ T cells (CD45+, CD3+, CD4+) and NK cell (CD45+, CD3−, NK1.1+) activation by mFc-IL-10 with or without mIgG1, D265A Anti PD1 in tumor infiltrating lymphocytes (TILs) from MC-38 mouse model was measured using flow cytometry as percent positive of Ki67 and Granzyme B. The flow cytometry panel was as follows: CD45 (30-F11; ThermoFisher), CD3 (145-2C11; Biolegend), CD4 (RM4-5; Biolegend), CD8 (53-6.7; ThermoFisher), NK1.1 (PK136; Biolegend), Ki67 (16A8; Biolegend), Granzyme B (NGZB; ThermoFisher), IFNγ (XMG1.2; Biolegend), and Viability dye (ThermoFisher). Ki67 levels correlate with proliferation while Granzyme B levels correlate with activation of CD8⁺ T cells and NK cells in TIL.

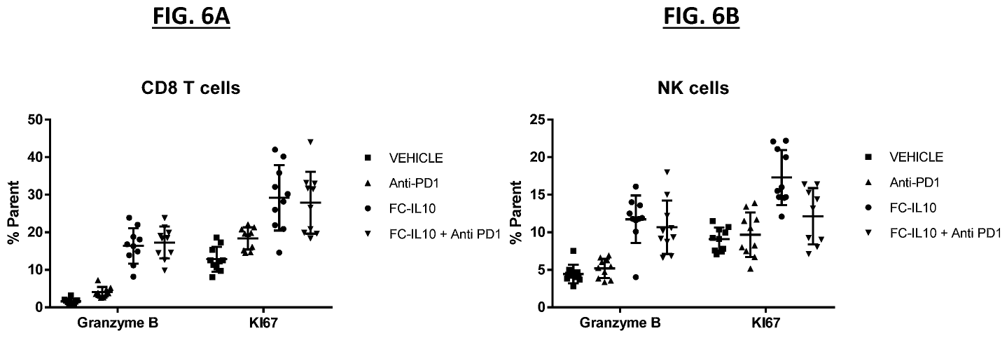

Briefly, mouse CRC tumors (MC-38) from the tumor bearing mice were collected 5 days post treatment. The tumors were enzymatically digested using a Tumor dissociation kit (Miltenyi). Dissociated cells were filtered through a 70 μm filter, stimulated with PMA and ionomycin in the presence of Brefeldin A, a protein transport inhibitor, washed with Dulbecco's phosphate-buffered saline (DPBS) (Gibco) and stained with a viability dye. Samples were subsequently blocked for non-specific binding (BD mouse FC block, BD Biosciences), stained for external markers (CD45, CD8, NK1.1), fixed and permeablized using FOXp3/Transcription Factor Stain Buffer Set (E-Bioscience) and stained for internal markers (CD3, Ki67, Granzyme B, and IFNγ). After a 30-minute incubation, samples were washed, re-suspended in FACS buffer (Dulbecco's phosphate-buffered saline (DPBS) (Gibco)+0.5% Fetal Bovine Serum (FBS) (Gibco) and analyzed by FACSCantoX™ flow cytometry system. Treatment with Fc-IL-10 leads to increased activation and proliferation of CD8⁺ T cells and NK cells in TIL (FIGS. 6A-6B).

CT-26 Treated with mFc-IL-10 in Combination with Anti-PD1:

CT-26 tumor bearing Balb/CAnNHsd female mice received 0.1, 0.3 or 1.0 mg/kg mFc-IL-10, QDx1 IP, or 1.0 mg/kg of isotype control anti-diphtheria toxin (anti-DT) mIgG1, QDx1 IP, on day 7. Mice also received, in combination, 10 mg/kg anti-PD1 mIgG1 D265A (“anti-PD1”), IP Q4Dx3 (dosed intraperitoneally once every 4 days for 3 doses), or 10 mg/kg of isotype control anti-DT mIgG1, IP Q4Dx3, beginning on day 7. Individual tumor volumes measured and recorded.

Eight mice (n=8) per group were allocated for tumor volume efficacy; n=7 per group per collection day allocated for tumor immune-monitoring in specific groups on day 14, 21 and 28 (7, 14, and 21 days post initial dose, respectively). Blood for PK micro-sampling was taken from animals allocated for immune-monitoring.

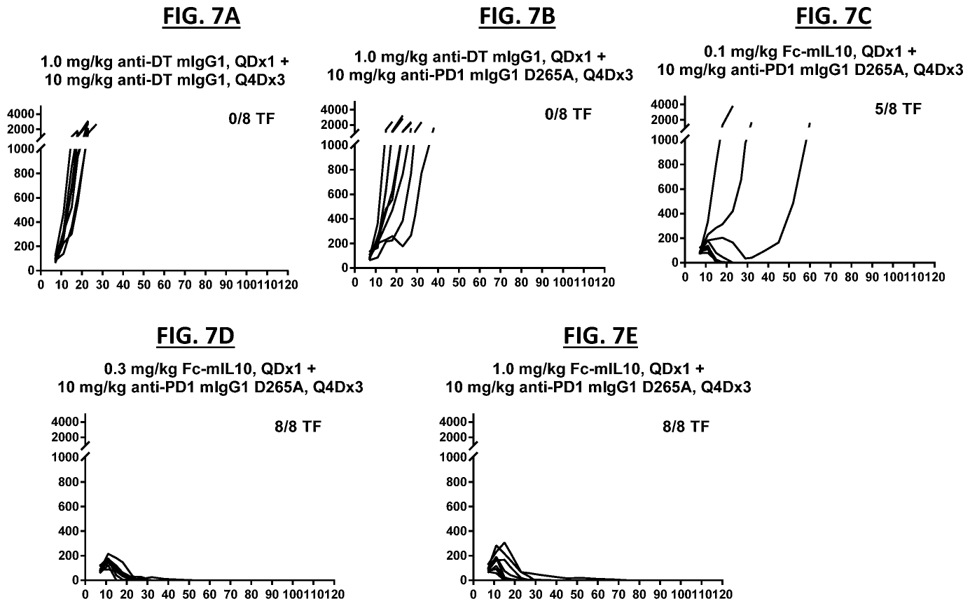

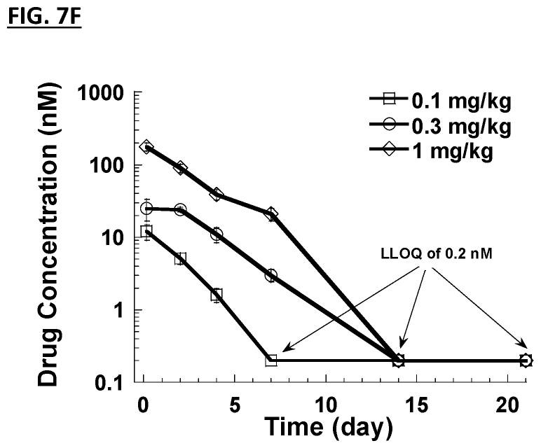

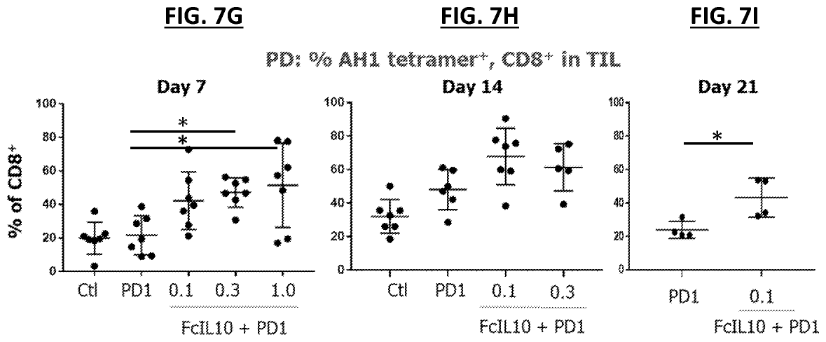

The number of tumor free mice yielded 0% from the control group; 0% (0/8), 62.5% (5/8), 100% (8/8), and 100% (8/8) from groups receiving 0, 0.1, 0.3, and 1.0 mg/kg of mFc-IL10, in combination with anti-PD1 mIgG1 D265A, respectively (FIGS. 7A-7E).

In CT26 tumor bearing mice treated with 0.1, 0.3 and 1 mg/kg mFc-IL-10, respectively, the detectable drug concentration in the circulation was below the LLOQ 14 days after dosing across different dose levels (FIG. 7F).

Immune Monitoring

Percentage of antigen specific CD8+ T cells in TILs from CT26 mouse model treated with mFc-IL-10+mIgG1, D265A Anti PD1 (“anti-PD1”) was measured using flow cytometry. The flow cytometry panel was as follows: CD45 (30-F11; ThermoFisher), CD3 (145-2C11; Biolegend), CD8 (53-6.7; ThermoFisher), AH1 Tetramer (MBL) and Viability dye (ThermoFisher).

Briefly, CT26 tumors from the tumor bearing mice were collected 7, 14 and 21 days post treatment and were manually dissociated using Miltenyi GentleMAC Octo Dissociator. Dissociated cells were filtered through a 70 μm filter and stimulated with PMA and ionomycin in the presence of a protein transport inhibitor cocktail. Samples are then washed with Dulbecco's phosphate-buffered saline (DPBS) (Gibco) and stained with a viability dye. Samples were blocked for non-specific binding (BD mouse FC block, BD Biosciences), stained for AH1 tetramer followed by incubation with antibodies (against CD45, CD3, and CD8, respectively). After a 30-minute incubation, samples were washed, re-suspended in FACS buffer (Dulbecco's phosphate-buffered saline (DPBS) (Gibco)+0.5% Fetal Bovine Serum (FBS) (Gibco) and analyzed by FACSCantoX™ flow cytometry system. Tumor specific AH1 tetramer positive CD8+ T cells were increased in CT26 tumors of mice treated with mFc-IL-10 in combination of anti-PD1 as compared to anti-PD1 treatment alone (FIGS. 7G-7I).

CT-26 Treated with mFc-IL-10 or PEG-mIL10 in Combination with Anti-PD1

CT-26 tumor bearing Balb/CAnNHsd female mice received 0.03, 0.1 or 0.3 mg/kg mFc-IL10, a single IP administration (QDx1 IP), or 0.2 or 1.0 mg/kg 5 kD PEG-mIL10, QDx25 SC (administered daily for 25 days, subcutaneous), or 3.0 mg/kg 5 kD PEG-mIL10, QDx1 SC (a single subcutaneous administration), beginning on day 7. Mice also received, in combination, 10 mg/kg anti-PD1 mIgG1 D265A, IP Q4Dx3, or 10 mg/kg isotype control anti-DT mIgG1, IP Q4Dx3, beginning on day 7. Individual tumor volumes measured and recorded.

Ten mice (n=10) per group allocated for tumor volume efficacy; n=6 per group allocated for blood and tumor immune-monitoring on day 14 (7 days post initial dose).

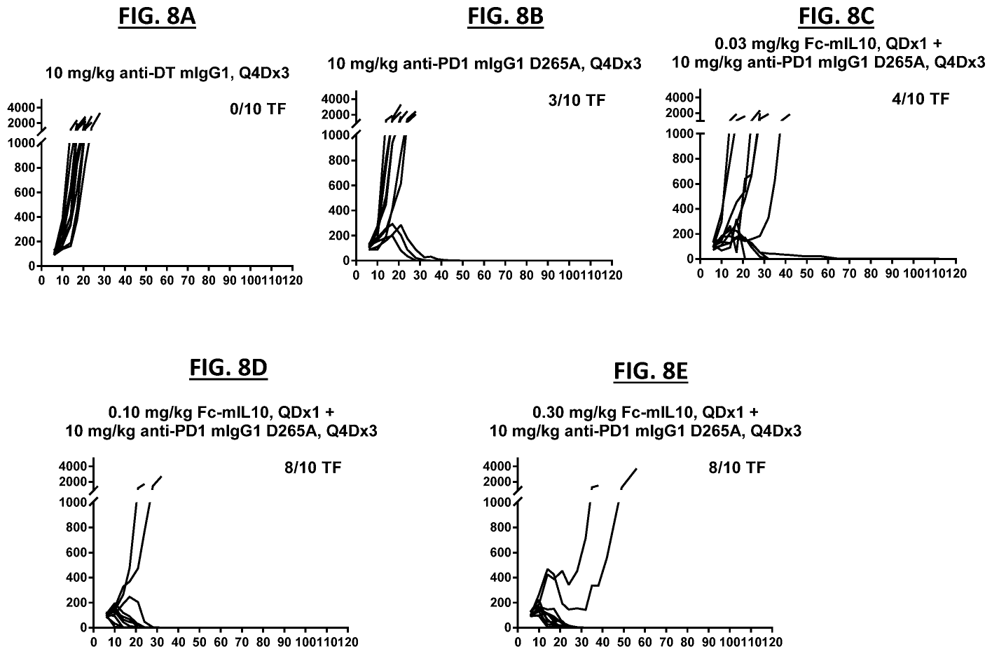

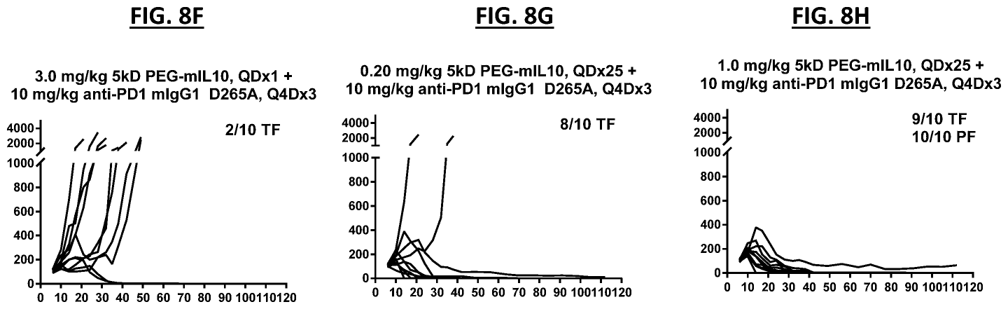

The number of tumor free mice yielded 0% (0/10) from the control group and 30% (3/10) from animals receiving only anti-PD1 mIgG1 D265A (FIGS. 8A-8E).

From groups receiving mFc-IL10, there resulted in a yield of 40% (4/10), 80% (8/10) and 80% (8/10) tumor free mice upon receiving a single IP administration of 0.03, 0.10 and 0.30 mg/kg Fc-mIL10, in combination with anti-PD1 mIgG1 D265A, respectively (FIGS. 8A-8E).

In comparison, when PEG-mIL10 was administered as a single subcutaneous dose at 3.0 mg/kg, in combination with anti-PD1 mIgG1 D265A, this resulted in only a 20% (2/10) yield of tumor free mice (FIG. 8F) (while in the control group of mice receiving only anti-PD1 mIgG1 D265A, 30% of the animals were tumor free). Upon receiving 25 daily subcutaneous administrations of 0.20 mg/kg PEG-mIL10, in combination with anti-PD1 mIgG1 D265A, there resulted in a yield of 80% (8/10) tumor free mice (FIG. 8G). A yield of 90% (9/10) tumor free, with an additional 10% progression free, resulted upon receiving 1.0 mg/kg PEG-mIL10, QDx25 SC, in combination with anti-PD1 mIgG1 D265A (FIG. 8H).

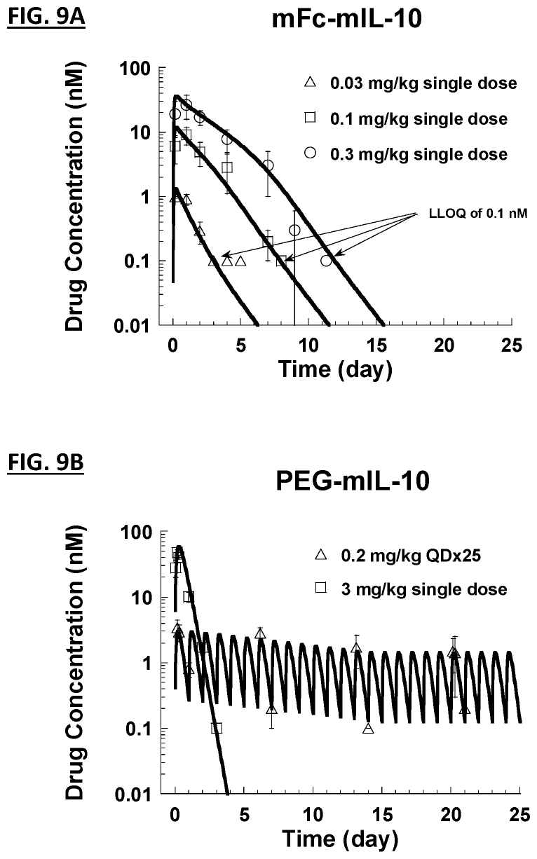

A comparison of drug concentration-time profiles of mFc-mIL-10 and pegylated mIL-10 in the above treated mice is shown in FIGS. 9A and 9B. The pharmacokinetic (PK) data of mFc-mIL-10 following intraperitoneal (IP) administration to mice were fitted with a three-compartment model coupled with first-order absorption using the SAAM II software (version 2.3.1, The Epsilon Group, Charlottesville, Va., USA). The schematic representation of the pharmacokinetic model for mFc-mIL-10 is shown below:

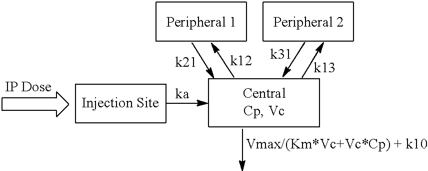

The differential equations used for the pharmacokinetic model are described as follows:

$$\frac{{dA}_{injection site}}{dt} = - {k}_{a} \times {A}_{injection site} \qquad ( 1 )$$

$${V}_{c} \frac{{dC}_{P}}{dt} = ka \times {A}_{injection site} + {k}_{21} \times {A}_{peripheral 1} + {k}_{31} \times {A}_{peripheral 2} - ( {k}_{10} + {k}_{12} + {k}_{13} + \frac{{V}_{max}}{{K}_{m} \times {V}_{c} + {V}_{c} \times {C}_{p}} ) \times {V}_{c} \times {C}_{p} \qquad ( 2 )$$

$$\frac{{dA}_{peripheral 1}}{dt} = {k}_{12} \times {V}_{c} \times {C}_{p} - {k}_{21} \times {A}_{peripheral 1} \qquad ( 3 )$$

$$\frac{{dA}_{peripheral 2}}{dt} = {k}_{13} \times {V}_{c} \times {C}_{p} - {k}_{31} \times {A}_{peripheral 2} \qquad ( 4 )$$

where Cp is the drug concentration in the central compartment; Vc is the volume of distribution in the central compartment; A_{injection site}, A_{peripheral 1}, and A_{peripheral 2} are the amount of drug in the injection site, peripheral compartment no. 1 and 2, respectively; ka is the absorption rate constant; k12, k21, k13, and k31 are the transfer rate constant between the central and the peripheral compartments; k10 is the non-target-mediated elimination rate constant; Vmax is the maximum target-mediated elimination rate constant; Km is the Michaelis-Menten constant at which the target-mediated elimination rate constant is half of the Vmax. At time zero, A_{injection site} is equal to the dose administered, with drug amounts or concentrations in the rest of compartments equal to zero. To aid the parameter estimation, the Bayesian estimation was used for the Vmax and Km determination.

The PEG-mIL-10 PK data after daily subcutaneous (SC) dosing were fitted with a one-compartment model along with first-order absorption using the SAAM II software. The schematic representation of the pharmacokinetic model for PEG-mIL-10 is shown below:

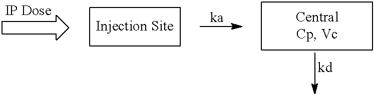

The differential equations used for the pharmacokinetic model are listed as follows:

$$\frac{{dA}_{injection site}}{dt} = - {k}_{a} \times {A}_{injection site} \qquad ( 5 )$$

$${V}_{c} \frac{{dC}_{P}}{dt} = {k}_{a} \times {A}_{injection site} - {k}_{d} \times {V}_{c} \times {C}_{p} \qquad ( 6 )$$

where kd is the elimination rate constant and was fitted individually for the 0.2-mg/kg SC daily dose group and the 3-mg/kg single dose. At time zero, A_{injection site} is equal to the dose administered, with drug concentrations in the central compartment equal to zero. To account for the reduction in the exposure to PEG-mIL-10 upon daily dosing, the dose was adjusted using the following equations: daily dose=nominal value of dose×e^{-0.002375×time in hours} from time zero to two weeks; daily dose=nominal value of dose×e^{-0.002375×336 hours} from two weeks onwards. Goodness of fit was assessed by the minimization of the objective function, Akaike and Schwarz-Bayesian information criteria, visual inspection of the fitting and residual plots, and the precision of the parameters estimated.

PEG-mIL-10 was pharmacologically active when it was administered at 0.2 mg/kg SC daily for 25 days and the drug concentration in the circulation was maintained throughout the treatment process; however, it was inactive at a single SC dose at 3 mg/kg while the drug concentration dropped below the LLOQ by day 4. In contrast, mFc-mIL-10 was pharmacologically active when administered single IP dose at 0.1 or 0.3 mg/kg, even though the drug concentrations decreased to the LLOQ by 8 and 12 days post dosing, respectively.

### Example 6. Additional Mouse Tumor Models

The mouse surrogate Fc-IL-10 molecule, mFc-IL-10, was tested in multiple mouse tumor models as a monotherapy or in combination with checkpoint blockade. Therapeutic dosing begins at tumor volume of ˜100 mm³, while prophylactic dosing begins 1-2 days prior to tumor implant. mFc-IL-10 showed robust single dose anti-tumor efficacy as a monotherapy in J558L myeloma and 1956 sarcoma, in addition to MC38 and CT26, models as well as significant single-dose activity when combined with checkpoint blockade in more resistant tumor models, including B16-F10 and 4T1 (Table 6).

TABLE 6

| | Single Agent Activity | Combination Results |
|---|---|---|
| Tumor Models | (therapeutic dosing unless noted) | (plus anti-PDl or anti-CTLA4) |
| J558L myeloma (SC) | Very potent single dose activity | NA |
| | (n = 2) | |
| 1956 sarcoma (SC) | Very potent single dose activity | NA |
| | (n = 2) | |
| | Activity remained when dosing | |
| | was delayed - larger tumors | |
| | (n = 1) | |
| MC38 colorectal (SC) | Very potent single dose activity | NA |
| | (n = 8) | |
| | Very potent activity when dosed | |
| | prophylactically (n = 1) | |
| CT26 colorectal (SC) | Very potent single dose activity | Very potent activity when combined |
| | (n = 8) | with anti-CTLA4 IgG2b (n = 2) |
| | Very potent activity when dosed | Very potent activity when combined |
| | prophylactically (n = 1) | with anti-PD1, and combination with |
| | | anti-PD1 lowers dose required for |
| | | efficacy (n = 6) |
| B16 melanoma (SC) | Marginal activity (n = 5) | Significant activity when combined |
| | | with anti-CTLA4 IgG2a (1 of 2 exps |
| | | n = 2) |
| | | Significant activity when combined |
| | | with anti-PD1 (2 of 2 exps., n = 2) |
| B16 melanoma (IV, met) | Significant single dose activity | NA |
| | (n = 2) | |
| 4T1 breast (SC) | Marginal activity (n = 1) | Significant activity when combined |
| | | with anti-CTLA4 IgG2a (n = 1) |
| | | Significant activity when combined |
| | | with anti-PD1 (n = 1) |
| AOM/DSS chemically- | Significant single agent activity, | Reversed anti-CTLA4 induced body |
| induced CRC | reduced induced colitis and | weight loss (2 of 2 experiments, n = 2) |
| | reduced number of lesions (n = 2) | |

“Very potent” = 60-100% TF single dose @ 0.3-10.0 mg/kg or in combination with anti-CTLA4 or anti-PD1;
“Potent” = 40% TF single dose @ 0.3-10.0 mg/kg;
“Significant activity” = tumor growth delay, reduced # of lesions, but few, if any, mice rendered tumor free.

### Example 7. Fc-IL-10 and Anti-CTLA4 in Chemically Induced Colitis Model

Azoxymethane (AOM)/dextran sulfate sodium (DSS)-induced mice were used as a preclinical model of colitis-associated colorectal cancer (Thaker, A. I., et al., J Vis Exp., 2012; (67): 4100) to determine the role of immune checkpoint blockers, such as anti-CTLA4, and the effect of Fc-IL-10 treatment in the development of colitis. In this model, AOM/DSS treated mice develop chronic intestinal inflammation and adenocarcinoma of the colon.

To induce colitis and colon tumors, 8-week old C57Bl/6 mice were treated with a single Intraperitoneal dose of AOM (10 mg/kg) followed by three seven-day cycles of oral DSS (2.5% in distilled water) over a 10-week period (Thaker, A. I., et al., J Vis Exp., 2012; (67): 4100). At day 70, mice were randomized by bodyweight and treated with biologics (isotype control anti-DT mIgG1+anti-DT mIgG2a, anti-CTLA4, mFc-IL-10, or a combination thereof) every 5 days for a total of 6 injections. Percent weight loss relative to baseline was used as a surrogate measure of colitis severity. The mice having a weight loss of more than 20% or 2-3 days of diarrhea and rectal bleeding were considered as not meeting the survival endpoint and were euthanized. Surviving animals were collected at day 40 post treatment initiation. To determine the anti-tumor efficacy of treatments, the number and size of tumors in the colons of collected animals were evaluated.

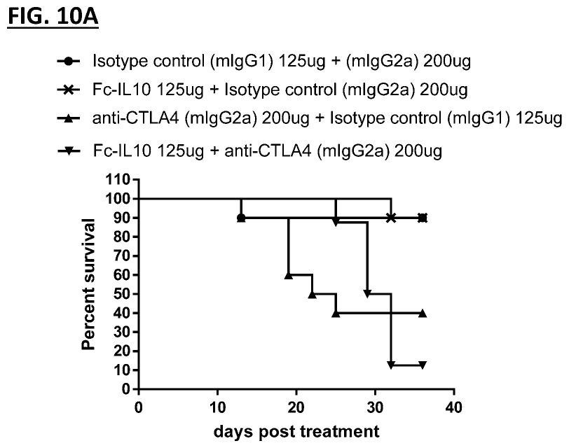

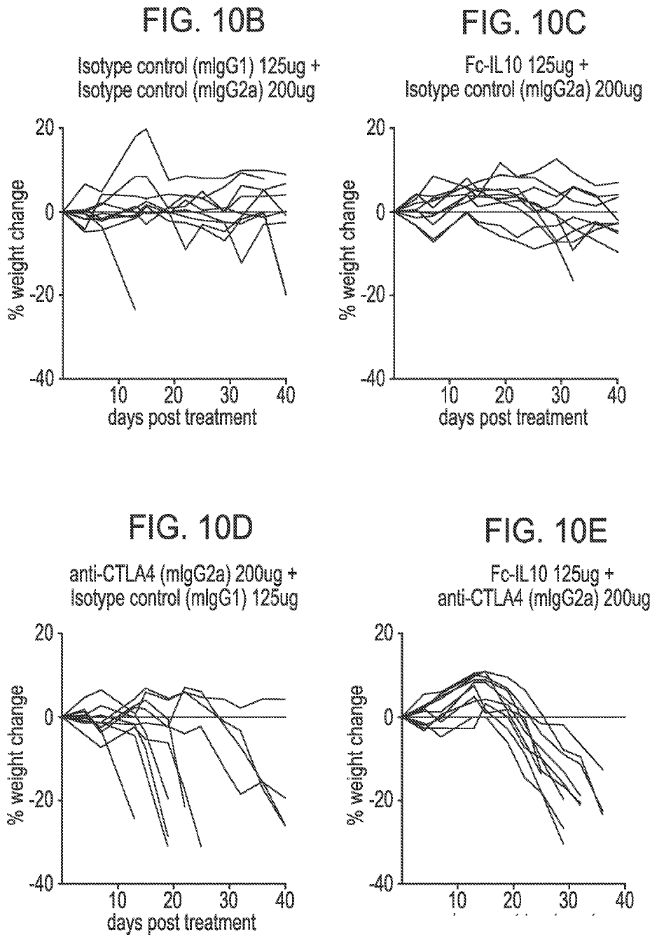

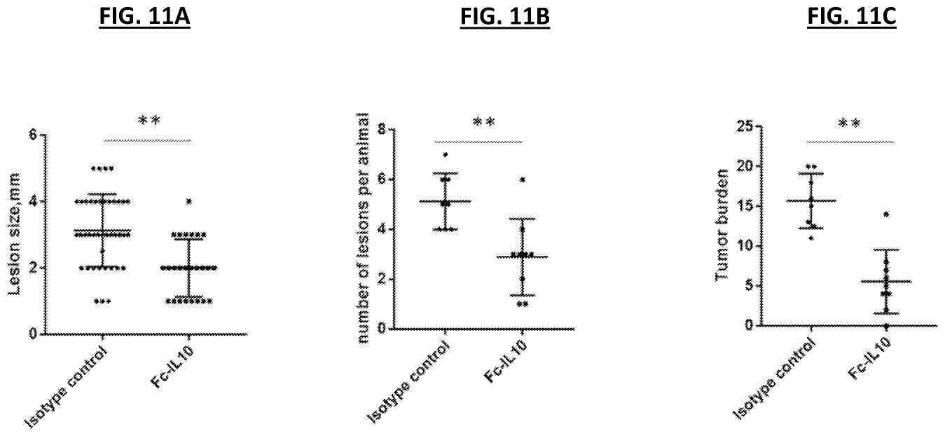

The mice treated with mFc-IL-10 showed similar survival and weight loss compared to the isotype control group (FIG. 10A). In the anti-CTLA4 treatment group, percent survival decreased quickly and more mice exhibited fast weight loss than the isotype control group. In comparison, the mice treated with a combination of anti-CTLA4 and mFc-IL-10 showed delayed onset of weight loss and increased survival until day 25 compared to the mice treated with only anti-CTLA4 (FIGS. 10A-10E). Thus, Fc-IL-10 alleviated anti-CTLA4 exasperation of colitis. Fc-IL-10 treatment also led to reduction of tumors of the colon in AOM/DSS-induced mice as compared to the isotype control (FIGS. 11A-11C).

### Example 8. Repeat Administration Studies in Cynomolgus Monkeys

Anemia & thrombocytopenia are anticipated adverse events associated with IL10 treatment (Fedorak, R. N., et al., Gastroenterology, 2000; 119: 1473-1482). Pegilodecakin (AM0010), a PEG-IL-10 currently tested in the clinic, is dosed on a schedule of 5 days on, 2 days off to avoid grade 3$ hemotologic adverse events (Hecht, J. R., et al., J. of Clinical Oncology, 2018; 36:4_suppl, 374-374).

As part of the safety assessment, Fc-IL-10 was administered in repeat dose studies to cynomolgus monkeys (N=3/dose) by subcutaneous (SC; Q weekly×3 at 0.1 and 0.3 mg/kg) or intravenous (IV) injection (Q 2 weekly×3 at 0.06 and 0.18 mg/kg or Q 4 week×2 at 0.06 mg/kg). The cynomolgus monkeys (Mauritius) of mixed gender and approximately 3 years of age study were randomly placed in dose groups and received either vehicle (20 mM Tris, 250 mM Sucrose, 0.05 mM DTPA, 0.05% Tween 80, pH 7.5) or Fc-IL-10 by SC injection into the dorsal midline between the scapulae or intravenous IV administration by slow bolus into the saphenous vein via an indwelling catheter. Animals were observed at least once during pretest, during the first hour post dose and at blood collection timepoints and/or daily for changes in condition and behavior. Blood samples were collected from the femoral vein for pharmacokinetics (0.5 mL in K₂EDTA tubes), hematology (1.0 mL in K₂EDTA tubes) or serum chemistry (1.5 mL in serum separator tubes) according to the schedule in Table 7. The toxicological observations for monkeys receiving BMS-986333 are listed in Table 8 and key changes in hematology are listed in Table 9. In summary, in monkeys receiving Q weekly Fc-IL-10, there were dose-limiting adverse findings in 1 of 3 animals at 0.1 (Hct 0.5× and platelets 0.4× as compared to predose) and at 0.3 mg/kg (Hct 0.15× and platelets 0.06× as compared to predose) with mortality at 0.3 mg/kg. Fc-IL-10 administered to monkeys at less frequent intervals (Q2 or Q4 weekly) at 0.06 mg/kg or Q2 weekly at 0.18 mg/kg was better tolerated with a moderate effect on the RBC parameters and platelets (Hct 0.65× and platelets 0.22-0.3× as compared to predose).

TABLE 7
Schedule of blood collection for assessment of pharmacokinetics, hematology and
serum chemistry in cynomolgus monkeys receiving Fc-IL-10.

| Study Design | Dose (mg/kg) | Pharmacokinetics | Hematology (CBC)* | Serum Chemistry** |
|---|---|---|---|---|
| Q weekly × 3 | 0.1 or 0.3 | 24, 96, 168 after 1st | Pretest, Day 8 | Pretest, Day 22 and |
| | | dose and predose, 24, | (predose), Day 11, | Day 29. |
| | | 96, and 168 hrs after | Day 15 (predose), | |
| | | 3rd dose | Day 22 and Day 29 | |
| Q 2 weekly × 3 | 0.06 or 0.18 | Predose, 10 min, 1, 3, | Predose, 24, 48, 72, | Pretest, 24 and 168 |
| | | 7, 24, 48, 72, 96, post | 96 and 192 hr post | hr post 3rd dose |
| | | 1st and 3rd doses | 1st dose; predose, | |
| | | | 24, 48, 72, 96, 168, | |
| | | | 240, 336 hr post 3rd | |
| | | | dose | |
| Q 4 weekly × 2 | 0.06 | Predose, 10 min, 1, 3, | Predose, 24, 48, 72, | Pretest, 24 and 168 |
| | | 7, 24, 48, 72, and 168 | 96 and 192 hr post | hr post 2nd dose |
| | | post 1st dose, | 1st dose; predose, | |
| | | predose, 1, 24, and | 24, 48, 72 and 96 hr | |
| | | 168 hr post 2nd dose. | post 3rd dose. | |

*CBC = white blood cell, red blood cell, hemaglobin, hematocrit, mean corpuscular volume, mean corpuscular hemaglobin content, red cell distribution width, platelet and reticulocyte counts with differentials

**Serum Chemistry=Aspartate aminotransferase, alanine amino transferase, alkaline phosphatase, gamma glutamyl transferase, total bilirubin, blood urea nitrogen, glucose, calcium, triglycerides, albumin, total protein, globulin, A/G ratio, inorganic phos, creatinine, cholesterol, sodium, potassium and chloride.

TABLE 8
Summary of toxicology observations of Fc-IL-10 following repeat
administration to cynomolgus monkeys.

| Study Design | Key Results |
|---|---|
| Repeat (QW × 3) SC | Dose-limiting adverse findings in 1 of 3 monkeys at 0.1 and 0.3 mg/kg. |
| tolerability study in | Mortality at 0.3 mg/kg. |
| cynomolgus monkeys at | 0.1 mg/kg: ↓ Hct (0.5× predose); 4/ Pit (0.4x predose) |
| 0.1 and 0.3 mg/kg; N = 3/ | Cmax (1^{st} dose) = 17nM; AUC (1^{st} dose) = 587 nM.h; |
| dose | 0.3 mg/kg: (1^{st} dose) Cmax = 35 nM; AUC = 2707 nM.h; |
| | ↓ Hct (0.15× predose) and ↓ platelets (0.06× predose). Mortality in 1 of 3 |
| | animals (euthanized). Necropsy findings: |
| | Spleen-marked splenic erythrophagocytosis and hemosiderosis. |
| | Lymph node-moderate lymphoid hyperplasia with medullary |
| | erythrophagocytosis and hemosiderosis |
| | Bone marrow-Decreased M:E ratio with increased erythrocyte precursors. |
| | Moderate erythrophagocytosis and hemosiderosis |
| | Liver-mild Kuppfer cell hyperplasia with hemosiderosis |
| | Heart, lung, skin, small intestine and colon: no significant histologic |
| | changes. |
| | Recovery in Hct and platelet counts within ≤ 3 weeks in surviving animals |
| Repeat IV tolerability | Well tolerated. No effects on clinical signs or body weights. |
| study in cynomolgus | 0.06 mg/kg: ↓Hct (0.8× vs predose) and ↓platelets (0.25× vs predose) |
| monkeys (Q 2 week × 3) | Cmax (1^{st} dose) = 33 nM; AUC (1^{st} dose) = 406 nM.h |
| at 0.06 mg/kg and 0.18 | 0.18 mg/kg: ↓ Hct (0.65× vs predose) and platelets (0.22× vs predose) |
| mg/kg; (Q 4 week × 2) at | Cmax (1^{st} dose) = 34 nM; AUC (1^{st} dose) = 1166 nM.h |
| 0.06 mg/kg; N = 3/ dose. | Recovery in Hct and platelet counts within ≤ 3 weeks. |

TABLE 9
Fc-IL-10: Effect of dosing frequency on hematocrit and platelet
counts in cynomolgus monkeys

| | | | | Pit count (× |
|---|---|---|---|---|
| Study Design | Dose (mg/kg) | Route | Het (× predose)* | predose)* |
| Q weekly × 3 | 0.1 | SC | 0.5× | 0.4× |
| | 0.3 | SC | 0.15× | 0.06× |
| Q 2 week × 3 | 0.06 | IV | 0.8× | 0.25× |
| | 0.18 | IV | 0.65× | 0.22× |
| Q 4 week × 2 | 0.06 | IV | 0.8× | 0.25× |

*Data reported for the lowest hematocrit (Hct) or platlet (Plt) count observed in the dose group.

Treatment-emergent ADA responses were detected in all monkeys dosed with Fc-IL-10 approximately 7-14 days post-dose which limited Fc-IL-10 exposure in the repeat administration studies and can have underestimated the impact of Fc-IL-10 on hematologic parameters.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

What is claimed:

1. An IL-10 fusion protein comprising (i) an IL-10 polypeptide, comprising an amino acid sequence having at least 95% sequence identity to the amino acid sequence set forth in SEQ ID NO: 1; and (ii) a second polypeptide, wherein the IL-10 fusion protein comprises an IL-10 activity.

2. The IL-10 fusion protein of claim 1, wherein the second polypeptide comprises an albumin polypeptide.

3. The IL-10 fusion protein of claim 1, wherein the second polypeptide comprises an Fc polypeptide.

4. The IL-10 fusion protein of claim 3, wherein the Fc polypeptide comprises an amino acid sequence having at least about 95% sequence identity to an amino acid sequence selected from SEQ ID NOs: 4-12.

5. The IL-10 fusion protein of any one of claims 1 to 4, wherein the second polypeptide is fused to the N-terminus of the IL-10 polypeptide.

6. The IL-10 fusion protein of any one of claims 1 to 5, wherein the second polypeptide is fused to the C-terminus of the IL-10 polypeptide.

7. The IL-10 fusion protein of any one of claims 1 to 6, wherein the IL-10 polypeptide is fused to the second polypeptide by a linker.

8. The IL-10 fusion protein of claim 7, wherein the linker comprises at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, or at least about 21 amino acids.

9. The IL-10 fusion protein of claim 7 or 8, wherein the linker comprises at least about 15 amino acids.

10. The IL-10 fusion protein of any one of claims 7 to 9, wherein the linker comprises at least about 20 amino acids.

11. The IL-10 fusion protein of any one of claims 7 to 10, wherein the linker comprises at least about 21 amino acids.

12. The IL-10 fusion protein of any one of claims 7 to 11, wherein the linker comprises a Glycine and a Serine.

13. The IL-10 fusion protein of any one of claims 7 to 12, wherein the linker comprises a GGGGS (SEQ ID NO: 39) motif or a GGGS (SEQ ID NO: 38) motif.

14. The IL-10 fusion protein of any one of claims 7 to 13, wherein the linker comprises an amino acid sequence selected from SEQ ID NOs: 38-45.

15. The IL-10 fusion protein of any one of claims 1 to 14, wherein the IL-10 polypeptide comprises an amino acid sequence having at least about 96%, at least about 97%, at least about 98%, or at least about 99% sequence identity to the amino acid sequence set forth in SEQ ID NO: 1.

16. The IL-10 fusion protein of any one of claims 1 to 15, wherein the IL-10 polypeptide comprises the amino acid sequence set forth in SEQ ID NO: 1.

17. The IL-10 fusion protein of any one of claims 3 to 16, wherein the Fc polypeptide comprises an amino acid sequence having at least about 96%, at least about 97%, at least about 98%, or at least about 99% sequence identity to an amino acid sequence selected from SEQ ID NOs: 4-12.

18. The IL-10 fusion protein of any one of claims 3 to 17, wherein the Fc polypeptide comprises an amino acid sequence selected from SEQ ID NOs: 4-12.

19. The IL-10 fusion protein of any one of claims 1, 3-5, and 7 to 18, comprising an amino acid sequence having at least 95% sequence identity to an amino acid sequence selected from SEQ ID NOs: 14-32.

20. The IL-10 fusion protein of any one of claims 1, 3-5, and 7 to 19, comprising an amino acid sequence having at least 98% sequence identity to an amino acid sequence selected from SEQ ID NOs: 14-32.

21. The IL-10 fusion protein of any one of claims 1, 3-5, and 7 to 20, comprising an amino acid sequence having at least 99% sequence identity to an amino acid sequence selected from SEQ ID NOs: 14-32.

22. The IL-10 fusion protein of any one of claims 1, 3-5, and 7 to 21, comprising an amino acid sequence selected from SEQ ID NOs: 14-32 with 3 or fewer substitutions, insertions, or deletions.

23. The IL-10 fusion protein of any one of claims 1, 3-5, and 7 to 22, comprising an amino acid sequence selected from SEQ ID NOs: 14-32 with 2 or fewer substitutions, insertions, or deletions.

24. The IL-10 fusion protein of any one of claims 1, 3-5, and 7 to 23, comprising an amino acid sequence selected from SEQ ID NOs: 14-32 with 1 substitution, insertion, or deletion.

25. The IL-10 fusion protein of any one of claims 1, 3-5, and 7 to 24, comprising an amino acid sequence selected from SEQ ID NOs: 14-32.

26. The IL-10 fusion protein of any one of claims 1, 3, 4, and 6 to 18, comprising an amino acid sequence having at least 95% sequence identity to an amino acid sequence selected from SEQ ID NOs: 33-36.

27. The IL-10 fusion protein of any one of claims 1, 3, 4, 6 to 18, and 26, comprising an amino acid sequence having at least 98% sequence identity to an amino acid sequence selected from SEQ ID NOs: 33-36.

28. The IL-10 fusion protein of any one of claims 1, 3, 4, 6 to 18, and 27, comprising an amino acid sequence having at least 99% sequence identity to an amino acid sequence selected from SEQ ID NOs: 33-36.

29. The IL-10 fusion protein of any one of claims 1, 3, 4, 6 to 18, and 27 to 28, comprising an amino acid sequence selected from SEQ ID NOs: 33-36 with 3 or fewer substitutions, insertions, or deletions.

30. The IL-10 fusion protein of any one of claims 1, 3, 4, 6 to 18, and 26 to 29, comprising an amino acid sequence selected from SEQ ID NOs: 33-36 with 2 or fewer substitutions, insertions, or deletions.

31. The IL-10 fusion protein of any one of claims 1, 3, 4, 6 to 18, and 26 to 30, comprising an amino acid sequence selected from SEQ ID NOs: 33-36 with 1 substitution, insertion, or deletion.

32. The IL-10 fusion protein of any one of claims 1, 3, 4, 6 to 18, and 26 to 31, comprising an amino acid sequence selected from SEQ ID NOs: 33-36.

33. The IL-10 fusion protein of any one of claims 1-32, wherein the IL-10 fusion protein is capable of treating cancer in a subject in need thereof when the IL-10 fusion protein is administered to the subject no more than once a week.

34. The IL-10 fusion protein of claim 33, wherein the IL-10 fusion protein is capable of treating cancer in a subject in need thereof when the IL-10 fusion protein is administered to the subject once about every two weeks.

35. The IL-10 fusion protein of claim 34, wherein the IL-10 fusion protein is capable of treating cancer in a subject in need thereof when the IL-10 fusion protein is administered to the subject once about every three weeks.

36. The IL-10 fusion protein of claim 35, wherein the IL-10 fusion protein is capable of treating cancer in a subject in need thereof when the IL-10 fusion protein is administered to the subject once about every four weeks.

37. The IL-10 fusion protein of claim 36, wherein the IL-10 fusion protein is capable of treating cancer in a subject in need thereof when the IL-10 fusion protein is administered to the subject once about every five weeks.

38. The IL-10 fusion protein of claim 37, wherein the IL-10 fusion protein is capable of treating cancer in a subject in need thereof when the IL-10 fusion protein is administered to the subject once about every six weeks.

39. The IL-10 fusion protein of any one of claims 33 to 38, wherein the IL-10 fusion protein is admistered to the subject at a dose ranging from about 0.001 mg/kg to about 0.5 mg/kg.

40. The IL-10 fusion protein of any one of claims 33 to 39, wherein the IL-10 fusion protein is admistered to the subject at a dose ranging from about 0.01 mg/kg to about 0.05 mg/kg.

41. The IL-10 fusion protein of any one of claims 33 to 40, wherein the IL-10 fusion protein is admistered to the subject at a dose ranging from about 0.05 mg/kg to about 0.1 mg/kg.

42. The IL-10 fusion protein of any one of claims 33 to 41, wherein the IL-10 fusion protein is admistered to the subject at a dose ranging from about 0.1 mg/kg to about 0.2 mg/kg.

43. The IL-10 fusion protein of any one of claims 33 to 42, wherein the IL-10 fusion protein is admistered to the subject at a dose ranging from about 0.2 mg/kg to about 0.3 mg/kg.

44. A polynucleotide or a set of polynucleotides encoding the IL-10 fusion protein of any one of claims 1 to 43.

45. A vector or a set of vectors comprising the polynucleotide or the set of polynucleotides of claim 44.

46. The vector or the set of vectors of claim 45, which is a viral vector.

47. A host cell comprising the IL-10 fusion protein of any one of claims 1 to 43, the polynucleotide or the set of polynucleotides of claim 44, or the vector or the set of vectors of claim 45 or 46.

48. The host cell of claim 47, which is a mammalian cell.

49. The host cell of claim 47 or 48, which is selected from a Chinese hamster ovary (CHO) cell, an HEK293 cell, a BHK cell, a murine myeloma cell (NS0 and Sp2/0), a monkey kidney (COS) cell, a VERO cell, a fibrosarcoma HT-1080 cell, and a HeLa cell.

50. A pharmaceutical composition comprising the IL-10 fusion protein of any one of claims 1 to 43, the polynucleotide or the set of polynucleotides of claim 44, or the vector or the set of vectors of claim 45 or 46, and a pharmaceutically acceptable excipient.

51. A method of treating a cancer in a subject in need thereof, comprising administering to the subject an effective amount of the IL-10 fusion protein of any one of claims 1 to 43, the polynucleotide or the set of polynucleotides of claim 44, the vector or the set of vectors of claim 45 or 46, or the pharmaceutical composition of claim 50.

52. A method of killing a cancer cell in a subject in need thereof, comprising administering to the subject an effective amount of the IL-10 fusion protein of any one of claims 1 to 43, the polynucleotide or the set of polynucleotides of claim 44, the vector or the set of vectors of claim 45 or 46, or the pharmaceutical composition of claim 50.

53. A method of treating cancer in a subject in need thereof, comprising administering to the subject an effective amount of an IL-10 fusion protein at a dosing interval of at least about 7 days, wherein the IL-10 fusion protein comprises an IL-10 polypeptide and a second polypeptide, which comprises an albumin polypeptide or an Fc polypeptide.

54. A method of killing a cancer call in a subject in need thereof, comprising administering to the subject an effective amount of an IL-10 fusion protein at a dosing interval of at least about 7 days, wherein the IL-10 fusion protein comprises an IL-10 polypeptide and a second polypeptide, which comprises an albumin polypeptide or an Fc polypeptide.

55. The method of claim 53 or 54, wherein the second polypeptide is an albumin polypeptide.

56. The method of claim 53 or 54, wherein the second polypeptide is an Fc polypeptide.

57. The method of any one of claims 53 to 56, wherein the IL-10 fusion protein further comprises a linker.

58. The method of claim 57, wherein the linker comprises the linker according to any one of claims 8 to 14.

59. The method of any one of claims 51 to 58, wherein the IL-10 fusion protein is administered at a dosing interval of at least about 7 days, at least about 10 days, at least about 14 days, at least about 17 days, at least about 21 days, at least about 24 days, or at least about 28 days.

60. The method of any one of claims 51 to 59, wherein the IL-10 fusion protein is administered no more than once week.

61. The method of any one of claims 51 to 60, wherein the IL-10 fusion protein is administered no more than once every 2 weeks.

62. The method of any one of claims 51 to 61, wherein the IL-10 fusion protein is administered no more than once every 3 weeks.

63. The method of any one of claims 51 to 62, wherein the IL-10 fusion protein is administered no more than once every 4 weeks.

64. The method of any one of claims 51 to 63, wherein the IL-10 fusion protein is administered at a dosing interval of at least about 7 days to at least about 28 days.

65. The method of claim 64, wherein the IL-10 fusion protein is administered at a dosing interval of at least about 14 days.

66. The method of claim 64, wherein the IL-10 fusion protein is administered at a dosing interval of at least about 21 days.

67. The method of any one of claims 51 to 58, wherein the IL-10 fusion protein is administered about once a week.

68. The method of any one of claims 51 to 58, wherein the IL-10 fusion protein is administered once about every 2 weeks.

69. The method of any one of claims 51 to 58, wherein the IL-10 fusion protein is administered once about every 3 weeks.

70. The method of any one of claims 51 to 58, wherein the IL-10 fusion protein is administered once about every 4 weeks.

71. The method of any one of claims 51 to 58, wherein the IL-10 fusion protein is administered once every about 6 weeks.

72. The method of any one of claims 51 to 71, wherein the IL-10 fusion protein is administered at a dose ranging from about 0.001 mg/kg to about 0.5 mg/kg.

73. The method of any one of claims 51 to 71, wherein the IL-10 fusion protein is administered at a dose ranging from 0.01 mg/kg to 0.2 mg/kg.

74. The method of any one of claims 51 to 58, wherein the IL-10 fusion protein comprises an amino acid sequence having at least 99% sequence identity to an amino acid sequence selected from SEQ ID NOs: 14-32, and wherein the IL-10 fusion protein is administered no more than once a week.

75. The method of claim 74, wherein the IL-10 fusion protein comprises an amino acid sequence selected from SEQ ID NOs: 14-32.

76. The method of claim 74 or 75, wherein the IL-10 fusion protein comprises of SEQ ID NO: 14.

77. The method of any one of claims 74 to 76, wherein the IL-10 fusion protein is administered once about every 2 weeks.

78. The method of any one of claims 74 to 76, wherein the IL-10 fusion protein is administered once about every 3 weeks.

79. The method of any one of claims 74 to 76, wherein the IL-10 fusion protein is administered once about every 4 weeks.

80. The method of any one of claims 74 to 76, wherein the IL-10 fusion protein is administered once about every 6 weeks.

81. The method of any one of claims 51, 53, and 55 to 80, wherein the cancer comprises a tumor.

82. The method of any one of claims 51 to 81, wherein the cancer is selected from the group consisting of small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma (glioblastoma multiforme), cervical cancer, stomach cancer, bladder cancer, hepatoma (hepatocellular carcinoma), breast cancer, colon carcinoma, head and neck cancer (or carcinoma), head and neck squamous cell carcinoma (HNSCC), gastric cancer, germ cell tumor, pediatric sarcoma, sinonasal natural killer, melanoma, metastatic malignant melanoma, cutaneous or intraocular malignant melanoma, mesothelioma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, and combinations of said cancers.

83. The method of any one of claims 51 to 81, wherein the cancer is selected from acute leukemia (ALL), acute myelogenous leukemia (AML), chronic lymphocytic leukemia (CLL), and chronic myelogenous leukemia (CML), undifferentiated AML, myeloblastic leukemia, myeloblastic leukemia, promyelocytic leukemia, myelomonocytic leukemia, monocytic leukemia, erythroleukemia, megakaryoblastic leukemia, isolated granulocytic sarcoma, chloroma, Hodgkin's lymphoma (HL), non-Hodgkin's lymphoma (NHL), B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC), lymphoplasmacytoid lymphoma (LPL) with Waldenstrom's macroglobulinemia; myeloma, IgG myeloma, light chain myeloma, nonsecretory myeloma, smoldering myeloma (indolent myeloma), solitary plasmocytoma, multiple myeloma, chronic lymphocytic leukemia (CLL), hairy cell lymphoma; and any combinations of said cancers.

84. The method of any one of claims 51 to 81, wherein the cancer is selected from RCC, NSCLC, gastric cancer, SCCHN, and any combinations of said cancers.

85. The method of any one of claims 51 to 81, wherein the cancer is selected from melanoma, bladder cancer, pancreatic cancer, HCC, colon cancer, SCLC, mesothelioma, hepatocellular carcinoma, prostate cancer, multiple myeloma, and combinations of said cancers.

86. The method of any one of claims 51 to 85, further comprising administering to the subject a second anticancer therapy.

87. The method of claim 86, wherein the second anticancer therapy comprises a therapy selected from the group consisting of an immunotherapy, a chemotherapy, a CAR-T therapy, a gene therapy, a radiation therapy, a surgery, an agent that activates innate immune cells, an agent that enhances survival of NK and/or CD8+ T-cells, and any combination thereof.

88. The method of claim 86 or 87, wherein the second anticancer therapy comprises an effective amount of an antibody or an antigen-binding fragment thereof that specifically binds a protein selected from Inducible T cell Co-Stimulator (ICOS), MICA, MICB, CD137 (4-1BB), CD134 (OX40), NKG2A, CD27, CD38, CD73, CD96, Glucocorticoid-Induced TNFR-Related protein (GITR), SLAMF7, BCMA, CCR8, and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CEACAM-1, CD52, HER2, and any combination thereof.

89. The method of any one of claims 86 to 88, wherein the second anticancer therapy comprises an antibody or antigen-binding fragment thereof that specifically binds PD-1 (“an anti-PD-1 antibody”).

90. The method of claim 89, wherein the anti-PD-1 antibody comprises nivolumab or pembrolizumab.

91. The method of any one of claims 86 to 90, wherein the second anticancer therapy comprises an antibody or an antigen-binding fragment thereof that specifically binds PD-L1 (“an anti-PD-L1 antibody”).

92. The method of claim 91, wherein the anti-PD-L1 antibody is selected from atezolizumab, durvalumab, and avelumab.

93. The method of any one of claims 86 to 92, wherein the second anticancer therapy comprises an antibody or an antigen-binding fragment thereof that specifically binds CTLA-4 (“an anti-CTLA-4 antibody”).

94. The method of claim 93, wherein the anti-CTLA-4 antibody comprises tremelimumab or ipilimumab.

95. The method of any one of claims 86 to 94, wherein the second anticancer therapy comprises a chemotherapy selected from a proteasome inhibitor, an IMiD, a Bet inhibitor, an IDO antagonist, a platinum-based chemotherapy, a STING agonist, a NLRP3 agonist, a TLR7 agonist, and any combination thereof.

96. The method of any one of claims 86 to 95, wherein the second therapy comprises an agent elected from doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, Folinic acid (leucovorin), Fluorouracil (5-FU), Oxaliplatin (Eloxatin), FOLFOX, Paclitaxel, Docetaxel, vincristine (ONCOVIN®), fludarabine (FLUDARA®), thalidomide (THALOMID®), alemtuzumab (CAMPATH®, ofatumumab (ARZERRA®), everolimus (AFINITOR®, ZORTRESS®), and carfilzomib (KYPROLISTM).

97. The method of any one of claims 86 to 96, wherein the second anticancer therapy comprises an agent that enhances survival of NK and/or CD8+ T-cells selected from IL-2 and pegylated IL-2.

98. The method of any one of claims 86 to 97, further comprising administering to the subject a third anticancer therapy.

99. The method of claim 98, wherein the third anticancer therapy comprises an antibody or an antigen-binding fragment thereof that specifically binds CTLA-4 (“an anti-CTLA-4 antibody”).

100. The method of claim 99, wherein the second anticancer therapy comprises an anti-PD-1 antibody and the third anticancer therapy comrpsines an anti-CTLA-4 antibody.

101. The method of claim 99 or 100, wherein the anti-CTLA-4 antibody comprises tremelimumab or ipilimumab.

102. The method of claim 100 or 101, wherein the anti-PD-1 antibody is nivolumab or pembrolizumab.

103. A method of preparing an IL-10 fusion protein, comprising expressing the polynucleotide or the set of polynucleotides of claim 44 or the vector or the set of vectors of claim 45 or 46 in a host cell under suitable conditions.

104. The method of claim 103, further comprising collecting the IL-10 fusion protein.


SEQUENCE LISTING (45 sequences)

SEQ ID NO: 1 - protein, 160 aa, Artificial Sequence
  hIL-10 Protein
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1630 bp, Artificial Sequence
  hIL-10 Nucleotide
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 3 - protein, 18 aa, Artificial Sequence
  Signal Peptide
MHSSALLCCLVLLTGVRA

SEQ ID NO: 4 - protein, 226 aa, Artificial Sequence
  Fc(Ig1.3f)
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 227 aa, Artificial Sequence
  Fc IgG1f
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Artificial Sequence
  Fc IgG1a/z
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Artificial Sequence
  Fc IgG1 (L234A,L235E,G237A)
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Artificial Sequence
  Fc IgG1 (P238K)
DKTHTCPPCPAPLLEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 226 aa, Artificial Sequence
  Fc IgG1f sans Terminal K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 226 aa, Artificial Sequence; identical to SEQ ID NO: 4
  IgG1.3f sans Terminal K

SEQ ID NO: 11 - protein, 226 aa, Artificial Sequence
  Fc-IgG1 P238K sans Terminal K
DKTHTCPPCPAPLLEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 233 aa, Artificial Sequence
  Fc - 3 Cysteine bridge variant
VEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 6 aa, Artificial Sequence
  cysteine bridge
VEPKSC

SEQ ID NO: 14 - protein, 407 aa, Artificial Sequence
  Fc-IL-10
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - protein, 408 aa, Artificial Sequence
  Fc-K-IL-10
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 16 - protein, 406 aa, Artificial Sequence
  FC-wt-hIL10 4xG4s spacer
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - protein, 406 aa, Artificial Sequence
  ILFC-301 FC-IL10 G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 18 - protein, 385 aa, Artificial Sequence
  ILFC-3XX FC-IL10 G4Sx0
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 19 - protein, 391 aa, Artificial Sequence
  ILFC-312 FC-IL10 G4Sx1
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 20 - protein, 396 aa, Artificial Sequence
  ILFC-313 FC-IL10 G4Sx2
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 21 - protein, 391 aa, Artificial Sequence; identical to SEQ ID NO: 19
  ILFC-312 FC-IL10 G4Sx1

SEQ ID NO: 22 - protein, 396 aa, Artificial Sequence; identical to SEQ ID NO: 20
  ILFC-313 FC-IL10 G4Sx2

SEQ ID NO: 23 - protein, 401 aa, Artificial Sequence
  ILFC-314 FC-IL10 G4Sx3
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 24 - protein, 397 aa, Artificial Sequence
  ILFC-313ss FC-IL10 G4Sx2
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 402 aa, Artificial Sequence
  ILFC-314ss 4 FC-IL10 G4Sx3
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 26 - protein, 407 aa, Artificial Sequence; identical to SEQ ID NO: 14
  ILFC-301ss FC-IL10 G4Sx4

SEQ ID NO: 27 - protein, 406 aa, Artificial Sequence
  ILFC-302 FC-IL10 N279D G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSEDSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 28 - protein, 406 aa, Artificial Sequence
  ILFC-303 FC-IL10 N361D G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVDSLGENLKTLRLRLRRCHRFLPCENKSKAVEQVKNAFNKLQEKGIYKAMSEFDIFINYIEAYMTMKIRN

SEQ ID NO: 29 - protein, 406 aa, Artificial Sequence
  ILFC-304 FC-IL10 N401D G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKDAFNKLQEKGIYKAMSEFDIFINYIEAYMTMKIRN

SEQ ID NO: 30 - protein, 406 aa, Artificial Sequence
  ILFC-305 FC-IL10 N385D G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCEDKSKAVEQVKNAFNKLQEKGIYKAMSEFDIFINYIEAYMTMKIRN

SEQ ID NO: 31 - protein, 406 aa, Artificial Sequence
  ILFC-306 FC-IL10 N353D G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAEDQDPDIKAHVNSLGENLKTLRLRLRRCHRFLPCENKSKAVEQVKNAFNKLQEKGIYKAMSEFDIFINYIEAYMTMKIRN

SEQ ID NO: 32 - protein, 405 aa, Artificial Sequence
  ILFC-307 FC-IL10 N435x G4Sx4
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIR

SEQ ID NO: 33 - protein, 397 aa, Artificial Sequence
  IL10-Fc G4Sx1
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 402 aa, Artificial Sequence
  IL10-Fc G4Sx2
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSVEPKSCDKTHTCPPCPAPEAEGAPSVFLFPPKPKDTLMISRTPEVTCVVVDVSHEDPEVKFNWYVDGVEVHNAKTKPREEYQNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 407 aa, Artificial Sequence
  IL10-Fc G4Sx3
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 412 aa, Artificial Sequence
  IL10-Fc G4Sx4
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - skipped

SEQ ID NO: 38 - protein, 4 aa, Artificial Sequence
  linker
GGGS

SEQ ID NO: 39 - protein, 5 aa, Artificial Sequence
  linker
GGGGS

SEQ ID NO: 40 - protein, 6 aa, Artificial Sequence
  linker
GGGGGS

SEQ ID NO: 41 - protein, 21 aa, Artificial Sequence
  linker
GGGGSSGGGGSGGGGSGGGGS

SEQ ID NO: 42 - protein, 20 aa, Artificial Sequence
  linker 4xG4s
GGGGSGGGGSGGGGSGGGGS

SEQ ID NO: 43 - protein, 15 aa, Artificial Sequence
  linker 3xG4s
GGGGSGGGGSGGGGS

SEQ ID NO: 44 - protein, 10 aa, Artificial Sequence
  linker 2xG4s
GGGGSGGGGS

SEQ ID NO: 45 - protein, 11 aa, Artificial Sequence
  Linker
GGGGSSGGGGS